(19) United States
(12) Patent Application Publication
(10) Pub. No.: US 2024/0285724 A1
(43) Pub. Date: Aug. 29, 2024

(54) METHODS AND COMPOSITIONS FOR TREATING OCULAR DISORDERS AND DISEASES

(71) Applicant: ONL Therapeutics, Inc., Ann Arbor, MI (US)

(72) Inventors: Andrew J. KOCAB, Farmington Hills, MI (US); Constance l. CHANG, Ann Arbor, MI (US); David N. ZACKS, Ann Arbor, MI (US); David A. ESPOSITO, Ann Arbor, MI (US); William J. BROCK, Ann Arbor, MI (US); Jana VAN DE GOOR, Ann Arbor, MI (US)

(21) Appl. No.: 18/570,576

(22) PCT Filed: Jun. 14, 2022
(86) PCT No.: PCT/US2022/033470

§ 371 Date: Dec. 14, 2023

Related U.S. Application Data
(60) Provisional application No. 63/210,935, filed on Jun. 15, 2021.

(51) Int. Cl. A61K 38/10; A61K 9/00

(52) U.S. Cl. CPC A61K 38/10; A61K 9/0048

(57) ABSTRACT

Treatment of chronic eye conditions further aims to prevent or delay the onset of irreversible vision impairment. Provided herein are methods and compositions for treating ocular diseases, disorders, and conditions. Described are compositions and methods for preventing, treating, or ameliorating an inflammation-mediated disease or condition in an individual comprising administering to the subject a Fas inhibitor, its derivative, or a pharmaceutically acceptable salt thereof.

### CROSS-REFERENCE

This application claims the benefit of U.S. Provisional Patent Application No. 63/210,935 filed Jun. 15, 2021, which is herein incorporated by reference in its entirety for all purposes.

### BACKGROUND

Ocular disorders and diseases affect a substantive percentage of the world's population. More than 4.2 million Americans 40 years of age or older are either legally blind or are with low vision, for example, having a loss and/or reduction in visual acuity. The leading causes of blindness and low vision in the United States include primarily age-related eye diseases such as age-related macular degeneration, cataract, diabetic retinopathy, and glaucoma. Treatment of eye conditions resulting in blindness or vision loss targets curing as well as addressing symptoms and progression. Treatment of chronic eye conditions further aims to prevent or delay the onset of irreversible vision impairment.

Ocular inflammation and its related complications are causes of vision loss. Basic and clinical research support a prominent role for inflammation underlying the pathogenesis of a wide array of retinal diseases. Vision is dependent on maintaining the integrity of the structure of the retina, and changes in retinal homeostasis resulting from retinal inflammation may provide the basis for vision loss and/or retinal disease. A number of conditions such as aging, metabolic abnormalities, altered vascular perfusion, or degenerative genetic conditions may also initiate various inflammatory processes within the retina. In the case of retinal inflammation, a dysregulated and/or prolonged immune response may contribute to both the pathogenesis of retinal diseases as well as vision threatening symptoms.

### SUMMARY

Despite advances in therapeutics aimed at treating ocular disorders and preserving or improving vision, the burden resulting from ocular diseases and disorders continues to increase. Many challenges exist in the development of therapeutics for treating disease and disorders in the eye of an individual. In the context of small molecule or biologic therapies, these therapies are generally administered via injection directly into the eye (e.g., intravitreal injection). Short-lived pharmacokinetic properties (e.g., drug half-life) and pharmacodynamic properties (e.g., therapeutic effect) of the active pharmaceutical ingredient (API) in such small molecule or biologic therapies necessitate frequent and repeated injections into the eye of an individual undergoing treatment. The challenges associated with frequent and repeated injection into the eye can result in further challenges and problems in treating a disease and disorder in the eye. For example, repeated injections into the eye can be detrimental to the overall health of the eye (e.g., an increased intraocular pressure and increased risk of developing glaucoma, among others), and repeating and frequent injections into the eye can also create barriers to treatment adherence because of the time (e.g., taking time off work, travel times associated with clinical visits, etc.) and financial commitment require to adhere to treatment programs associated with active pharmaceutical ingredients (API) having short-lived pharmacokinetic properties. Further, repeated injections into the eye can thwart or reduce compliance in general because an individual in need of a therapy is less likely, in. certain instances, to pursue a therapy requiring repeated injections into the eye. Therefore, therapeutics having an extended persistence in an eye (e.g., extended pharmacokinetic properties) and dosing programs that do not require frequent injections into the eye are advantageous for treating disease and disorders. The methods and compositions described herein provide a solution to such challenge.

Accordingly, provided herein are methods and compositions for use in treating ocular diseases and disorders that do not require frequent injection into the eye (e.g., less than once a month, once every three to six months, etc.). For example, Fas inhibitors (e.g., Fas inhibitor peptides) for inhibiting, reducing, and/or preventing Fas-mediated inflammation used in the methods and compositions described herein have an extended half-life in the vitreous humor, thereby enabling infrequent injections into the eye of an individual (e.g., an extended period between doses). By achieving an extended half-life in the vitreous humor, the vitreous humor provides, in an embodiment, a depot for supplying the retina with the Fas inhibitors described herein over an extended period (e.g., the period between a first dose and second dose or between each subsequent dose). The described Fas inhibitors for inhibiting, reducing, and/or preventing Fas-mediated inflammation are also useful for treating ocular diseases and disorders (e.g., retinal degeneration and/or the symptoms associated with retinal degeneration).

Provided and described herein are methods, comprising: administering a composition comprising a peptide comprising an amino acid sequence HHIYLGAVNYIY or a variant sequence thereof, or a pharmaceutically-acceptable salt of the peptide, to a vitreous humor of an eye, thereby providing the peptide to retinal tissue in the eye, whereby the peptide has a half-life in the vitreous humor of at least about (z) days (e.g., 30 to 300).

In some embodiments, the method is a method of treating inflammation (e.g., Fas-mediated inflammation) in an eye (e.g., the retinal tissue of an eye). In some embodiments, the method is a method of inhibiting, reducing, and/or preventing inflammation (e.g., Fas-mediated inflammation) in an eye (e.g., the retinal tissue of an eye). In some embodiments, the method includes treating symptoms associated with inflammation in the eye (e.g., increased intraocular pressure, loss of and/or decrease in visual acuity, blurred vision, distorted vision, etc.). In some embodiments, retinal inflammation can be determined by observing the symptoms associated with inflammation in the eye (e.g., retinal degeneration, increased intraocular pressure, loss of and/or decrease in visual acuity, blurred vision, distorted vision, etc.) and/or by a biological assay detecting the presence of inflammatory molecules (e.g., inflammatory cytokines) in a sample (e.g., vitreous humor sample) taken from the eye.

In some embodiments, the method is a method of treating retinal cell loss (e.g., retinal pigment epithelial cells, retinal ganglion cells, and/or photoreceptors) in an eye (e.g., the retinal tissue of an eye). In some embodiments, the method is a method of inhibiting, reducing, and/or preventing retinal cell loss (e.g., retinal pigment epithelial cells, retinal ganglion cells, and/or photoreceptors) in an eye (e.g., the retinal tissue of an eye). In some embodiments, the method includes treating symptoms associated with retinal cell loss (e.g., retinal pigment epithelial cells, retinal ganglion cells, and/or photoreceptors) in the eye (e.g., retinal degeneration, increased intraocular pressure, loss of and/or decrease in visual acuity, blurred vision, distorted vision, etc.).

In some embodiments, the method is a method of treating retinal degeneration. In some embodiments, the method is a method of inhibiting, reducing, and/or preventing retinal degeneration. In some embodiments, the method includes treating symptoms associated with retinal degeneration (e.g., increased intraocular pressure, loss of and/or decrease in visual acuity, blurred vision, distorted vision, etc.). In some embodiments, retinal degeneration comprises anatomical degeneration. In some embodiments, retinal degeneration comprises the loss and/or death of retinal pigment epithelial cells, retinal ganglion cells, photoreceptors, or a combination thereof. In some embodiments, retinal degeneration comprises the loss and/or death of retinal pigment epithelial cells. In some embodiments, retinal degeneration comprises the loss and/or death of retinal ganglion cells. In some embodiments, retinal degeneration comprises the loss and/or death of photoreceptors. In some embodiments, retinal degeneration comprises the loss and/or death a combination of retinal pigment epithelial cells, retinal ganglion cells, and/or photoreceptors.

In some embodiments, the method is a method of treating an ocular disease, disorder, or condition. In some embodiments, the method is a method of inhibiting, reducing, and/or preventing an ocular disease, disorder, or condition. In some embodiments, the method includes treating symptoms associated with an ocular disease, disorder, or condition (e.g., retinal degeneration, increased intraocular pressure, loss of and/or decrease in visual acuity, blurred vision, distorted vision, etc.). In some embodiments, the ocular disease, disorder, or condition is a chronic ocular disease, disorder, or condition. In some embodiments, the ocular disease, disorder, or condition comprises the loss and/or death of retinal pigment epithelial cells, retinal ganglion cells, photoreceptors, or a combination thereof. In some embodiments, the ocular disease, disorder, or condition comprises the loss and/or death of retinal pigment epithelial cells. In some embodiments, the ocular disease, disorder, or condition comprises the loss and/or death of retinal ganglion cells. In some embodiments, the ocular disease, disorder, or condition comprises the loss and/or death of photoreceptors. In some embodiments, the ocular disease, disorder, or condition comprises the loss and/or death a combination of retinal pigment epithelial cells, retinal ganglion cells, and/or photoreceptors.

In some embodiments, the method is a method of treating glaucoma. In some embodiments, the method is a method of inhibiting, reducing, and/or preventing glaucoma. In some embodiments, the method includes treating symptoms associated with glaucoma (e.g., retinal degeneration, increased intraocular pressure, loss of and/or decrease in visual acuity, blurred vision, distorted vision, etc.). In some embodiments, the ocular disease, disorder, or condition comprises the loss and/or death of retinal ganglion cells.

In some embodiments, the method is a method of treating macular degeneration. In some embodiments, the method is a method of inhibiting, reducing, and/or preventing macular degeneration. In some embodiments, the method includes treating symptoms associated with macular degeneration (e.g., retinal degeneration, increased intraocular pressure, loss of and/or decrease in visual acuity, blurred vision, distorted vision, etc.). In some embodiments, the macular degeneration is age-related macular degeneration, non-exudative age-related macular degeneration, or exudative age-related macular degeneration. In some embodiments, the macular degeneration is age-related macular degeneration. In some embodiments, the macular degeneration is non-exudative age-related macular degeneration. In some embodiments, the macular degeneration is exudative age-related macular degeneration. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors.

In some embodiments, the method is a method of treating retinal detachment. In some embodiments, the method is a method of inhibiting, reducing, and/or preventing retinal detachment. In some embodiments, the method includes treating symptoms associated retinal detachment (e.g., retinal degeneration, increased intraocular pressure, loss of and/or decrease in visual acuity, blurred vision, distorted vision, etc.). In some embodiments, the retinal detachment is chronic retinal detachment. In some embodiments, retinal detachment comprises the loss and/or death of retinal pigment epithelial cells, retinal ganglion cells, photoreceptors, or a combination thereof. In some embodiments, retinal detachment comprises the loss and/or death of retinal pigment epithelial cells. In some embodiments, retinal detachment comprises the loss and/or death of retinal ganglion cells. In some embodiments, retinal detachment comprises the loss and/or death of photoreceptors. In some embodiments, retinal detachment comprises the loss and/or death a combination of retinal pigment epithelial cells, retinal ganglion cells, and/or photoreceptors.

In some embodiments, the method is a method of treating a loss in visual acuity. In some embodiments, the method is a method of treating a loss in visual acuity. In some embodiments, a loss in visual acuity is characterized by and/or equivalent to a reduction in visual acuity (e.g., a reduction from 20/100 to 20/200, or a reduction from 20/40 to 20/50, etc.). In some embodiments, the method is a method of treating ocular hypertension (e.g., elevated intraocular pressure). In some embodiments, the method is a method of treating ocular hypertension (e.g., elevated intraocular pressure). In some embodiments, ocular hypertension is characterized by an intraocular pressures of 21 mm Hg or greater. In some embodiments, ocular hypertension includes by an intraocular pressures of 18 mm Hg or greater.

In some embodiments, the method is a method of treating a loss in visual function. In some embodiments, the method is a method of treating a loss in visual function. In some embodiments, a loss in visual function is characterized by and/or equivalent to a reduction in visual function assessments (e.g., visual acuity, field of vision, contrast sensitivity, binocular function, and/or light/dark adaptation).

In some embodiments, (z) is 30 to 300. In some embodiments, the method comprises using the vitreous humor as a depot to provide a therapeutically-effective amount of the peptide to the retinal tissue. In some embodiments, the peptide is provided to the retina from the vitreous humor for at least about (n) days after administration. In some embodiments, (n) is 30 to 365.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions.

In some embodiments, the peptide further comprises a modification. In some embodiments, modification comprises a modified amino acid. In some embodiments, the peptide comprises an amidated C-terminus. In some embodiments, the peptide has the structure of Formula III or a pharmaceutically-acceptable salt thereof.

In some embodiments, the peptide is detectable in the vitreous humor for at least about (z) days after administration. In some embodiments, the composition comprises the pharmaceutically-acceptable salt of the peptide. In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt. In some embodiments, the composition further comprises on or more excipients. In some embodiments, the composition further comprises a surfactant. In some embodiments, the surfactant is a non-ionic surfactant. In some embodiments, the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer. In some embodiments, the surfactant forms about 0.01% to about 20% weight/weight of the composition. In some embodiments, the surfactant forms about 0.05% to about 10% weight/weight of the composition. In some embodiments, the composition further comprises a tonicity adjusting agent, a buffering agent, or a combination thereof. In some embodiments, the composition is buffered at a pH of 2.5 to 7.5.

In some embodiments, the composition comprises 5 micrograms (ug) to 10,000 ug of the peptide. In some embodiments, the composition comprises at least 10 micrograms (ug), at least 25 ug, at least 50 ug, at least 100 ug, at least 150 ug, at least 200 ug, or at least 250 ug of the peptide. In some embodiments, the composition comprises 10 micrograms (ug), 25 ug, 50 ug, 100 ug, 150 ug, 200 ug, or 250 ug of the peptide. In some embodiments, the peptide is present at a concentration 0.1 milligrams per milliliter (mg/mL) to 10.0 mg/mL.

Provided and described herein are methods, comprising: (a) administering a first composition comprising a peptide comprising an amino acid sequence HHIYLGAVNYIY or a variant sequence thereof, or a pharmaceutically-acceptable salt of the peptide, to a vitreous humor of an eye; and (b) administering at least a second composition comprising the peptide to the vitreous humor of the eye, wherein the at least second dose is administered no less than about (y) days after the first administration.

In some embodiments, the method is a method of treating inflammation (e.g., Fas-mediated inflammation) in an eye (e.g., the retinal tissue of an eye). In some embodiments, the method is a method of inhibiting, reducing, and/or preventing inflammation (e.g., Fas-mediated inflammation) in an eye (e.g., the retinal tissue of an eye). In some embodiments, the method includes treating symptoms associated with inflammation in the eye (e.g., increased intraocular pressure, loss of and/or decrease in visual acuity, blurred vision, distorted vision, etc.). In some embodiments, retinal inflammation can be determined by observing the symptoms associated with inflammation in the eye (e.g., retinal degeneration, increased intraocular pressure, loss of and/or decrease in visual acuity, blurred vision, distorted vision, etc.) and/or by a biological assay detecting the presence of inflammatory molecules (e.g., inflammatory cytokines) in a sample (e.g., vitreous humor sample) taken from the eye.

In some embodiments, the method is a method of treating retinal cell loss (e.g., retinal pigment epithelial cells, retinal ganglion cells, and/or photoreceptors) in an eye (e.g., the retinal tissue of an eye). In some embodiments, the method is a method of inhibiting, reducing, and/or preventing retinal cell loss (e.g., retinal pigment epithelial cells, retinal ganglion cells, and/or photoreceptors) in an eye (e.g., the retinal tissue of an eye). In some embodiments, the method includes treating symptoms associated with retinal cell loss (e.g., retinal pigment epithelial cells, retinal ganglion cells, and/or photoreceptors) in the eye (e.g., retinal degeneration, increased intraocular pressure, loss of and/or decrease in visual acuity, blurred vision, distorted vision, etc.).

In some embodiments, the method is a method of treating retinal degeneration. In some embodiments, the method is a method of inhibiting, reducing, and/or preventing retinal degeneration. In some embodiments, the method includes treating symptoms associated with retinal degeneration (e.g., increased intraocular pressure, loss of and/or decrease in visual acuity, blurred vision, distorted vision, etc.). In some embodiments, retinal degeneration comprises the loss and/or death of retinal pigment epithelial cells, retinal ganglion cells, photoreceptors, or a combination thereof. In some embodiments, retinal degeneration comprises the loss and/or death of retinal pigment epithelial cells. In some embodiments, retinal degeneration comprises the loss and/or death of retinal ganglion cells. In some embodiments, retinal degeneration comprises the loss and/or death of photoreceptors. In some embodiments, retinal degeneration comprises the loss and/or death a combination of retinal pigment epithelial cells, retinal ganglion cells, and/or photoreceptors.

In some embodiments, the method is a method of treating an ocular disease, disorder, or condition. In some embodiments, the method is a method of inhibiting, reducing, and/or preventing an ocular disease, disorder, or condition. In some embodiments, the method includes treating symptoms associated with an ocular disease, disorder, or condition (e.g., retinal degeneration, increased intraocular pressure, loss of and/or decrease in visual acuity, blurred vision, distorted vision, etc.). In some embodiments, the ocular disease, disorder, or condition is a chronic ocular disease, disorder, or condition. In some embodiments, the ocular disease, disorder, or condition comprises the loss and/or death of retinal pigment epithelial cells, retinal ganglion cells, photoreceptors, or a combination thereof. In some embodiments, the ocular disease, disorder, or condition comprises the loss and/or death of retinal pigment epithelial cells. In some embodiments, the ocular disease, disorder, or condition comprises the loss and/or death of retinal ganglion cells. In some embodiments, the ocular disease, disorder, or condition comprises the loss and/or death of photoreceptors. In some embodiments, the ocular disease, disorder, or condition comprises the loss and/or death a combination of retinal pigment epithelial cells, retinal ganglion cells, and/or photoreceptors.

In some embodiments, the method is a method of treating glaucoma. In some embodiments, the method is a method of inhibiting, reducing, and/or preventing glaucoma. In some embodiments, the method includes treating symptoms associated with glaucoma (e.g., retinal degeneration, increased intraocular pressure, loss of and/or decrease in visual acuity, blurred vision, distorted vision, etc.). In some embodiments, the ocular disease, disorder, or condition comprises the loss and/or death of retinal ganglion cells.

In some embodiments, the method is a method of treating macular degeneration. In some embodiments, the method is a method of inhibiting, reducing, and/or preventing macular degeneration. In some embodiments, the method includes treating symptoms associated with macular degeneration (e.g., retinal degeneration, increased intraocular pressure, loss of and/or decrease in visual acuity, blurred vision, distorted vision, etc.). In some embodiments, the macular degeneration is age-related macular degeneration, non-exudative age-related macular degeneration, or exudative age-related macular degeneration. In some embodiments, the macular degeneration is age-related macular degeneration. In some embodiments, the macular degeneration is non-exudative age-related macular degeneration. In some embodiments, the macular degeneration is exudative age-related macular degeneration. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors.

In some embodiments, the method is a method of treating retinal detachment. In some embodiments, the method is a method of inhibiting, reducing, and/or preventing retinal detachment. In some embodiments, the method includes treating symptoms associated retinal detachment (e.g., retinal degeneration, increased intraocular pressure, loss of and/or decrease in visual acuity, blurred vision, distorted vision, etc.). In some embodiments, the retinal detachment is chronic retinal detachment. In some embodiments, retinal detachment comprises the loss and/or death of retinal pigment epithelial cells, retinal ganglion cells, photoreceptors, or a combination thereof. In some embodiments, retinal detachment comprises the loss and/or death of retinal pigment epithelial cells. In some embodiments, retinal detachment comprises the loss and/or death of retinal ganglion cells. In some embodiments, retinal detachment comprises the loss and/or death of photoreceptors. In some embodiments, retinal detachment comprises the loss and/or death a combination of retinal pigment epithelial cells, retinal ganglion cells, and/or photoreceptors.

In some embodiments, the method is a method of treating a loss in visual acuity. In some embodiments, the method is a method of treating a loss in visual acuity. In some embodiments, a loss in visual acuity is characterized by and/or equivalent to a reduction in visual acuity (e.g., a reduction from 20/100 to 20/200, or a reduction from 20/40 to 20/50, etc.). In some embodiments, the method is a method of treating ocular hypertension (e.g., elevated intraocular pressure). In some embodiments, the method is a method of treating ocular hypertension (e.g., elevated intraocular pressure). In some embodiments, ocular hypertension is characterized by an intraocular pressures of 21 mm Hg or greater. In some embodiments, ocular hypertension includes by an intraocular pressures of 18 mm Hg or greater.

In some embodiments, the method is a method of treating a loss in visual function. In some embodiments, the method is a method of treating a loss in visual function. In some embodiments, a loss in visual function is characterized by and/or equivalent to a reduction in visual function assessments (e.g., visual acuity, field of vision, contrast sensitivity, binocular function, and/or light/dark adaptation).

In some embodiments, (y) is 30 to 360. In some embodiments, (y) is 90 to 180. In some embodiments, the peptide has a half-life in the vitreous humor of at least about (z) days. In some embodiments, (z) is 30 to 300.

In some embodiments, the method comprises using the vitreous humor as a depot to provide the peptide to retinal tissue in the eye, whereby the peptide is present in the vitreous humor for at (n) days after administration. In some embodiments, the peptide is provided to the retina from the vitreous humor for at least about (n) days after administration. In some embodiments, (n) is 30 to 365.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions.

In some embodiments, the peptide further comprises a modification. In some embodiments, modification comprises a modified amino acid. In some embodiments, the peptide comprises an amidated C-terminus. In some embodiments, the peptide has the structure of Formula III or a pharmaceutically-acceptable salt thereof.

In some embodiments, the peptide is detectable in the vitreous humor for at least about (z) days after administration. In some embodiments, the composition comprises the pharmaceutically-acceptable salt of the peptide. In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt. In some embodiments, the composition further comprises on or more excipients. In some embodiments, the composition further comprises a surfactant. In some embodiments, the surfactant is a non-ionic surfactant. In some embodiments, the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer. In some embodiments, the surfactant forms about 0.01% to about 20% weight/weight of the composition. In some embodiments, the surfactant forms about 0.05% to about 10% weight/weight of the composition. In some embodiments, the composition further comprises a tonicity adjusting agent, a buffering agent, or a combination thereof. In some embodiments, the composition is buffered at a pH of 2.5 to 7.5.

In some embodiments, the composition comprises 5 micrograms (ug) to 10,000 ug of the peptide. In some embodiments, the composition comprises at least 10 micrograms (ug), at least 25 ug, at least 50 ug, at least 100 ug, at least 150 ug, at least 200 ug, or at least 250 ug of the peptide. In some embodiments, the composition comprises 10 micrograms (ug), 25 ug, 50 ug, 100 ug, 150 ug, 200 ug, or 250 ug of the peptide. In some embodiments, the peptide is present at a concentration 0.1 milligrams per milliliter (mg/mL) to 10.0 mg/mL.

Provided and described herein are methods, comprising: administering a plurality of compositions to a vitreous humor of an eye, the plurality of compositions being administered no more frequently than once every about (y) days, and each of the plurality of compositions comprising a peptide comprising an amino acid sequence HHIYLGAVNYIY or variant sequence thereof, or a pharmaceutically-acceptable salt of the peptide.

In some embodiments, the method is a method of treating inflammation (e.g., Fas-mediated inflammation) in an eye (e.g., the retinal tissue of an eye). In some embodiments, the method is a method of inhibiting, reducing, and/or preventing inflammation (e.g., Fas-mediated inflammation) in an eye (e.g., the retinal tissue of an eye). In some embodiments, the method includes treating symptoms associated with inflammation in the eye (e.g., increased intraocular pressure, loss of and/or decrease in visual acuity, blurred vision, distorted vision, etc.). In some embodiments, retinal inflammation can be determined by observing the symptoms associated with inflammation in the eye (e.g., retinal degeneration, increased intraocular pressure, loss of and/or decrease in visual acuity, blurred vision, distorted vision, etc.) and/or by a biological assay detecting the presence of inflammatory molecules (e.g., inflammatory cytokines) in a sample (e.g., vitreous humor sample) taken from the eye.

In some embodiments, the method is a method of treating retinal cell loss (e.g., retinal pigment epithelial cells, retinal ganglion cells, and/or photoreceptors) in an eye (e.g., the retinal tissue of an eye). In some embodiments, the method is a method of inhibiting, reducing, and/or preventing retinal cell loss (e.g., retinal pigment epithelial cells, retinal ganglion cells, and/or photoreceptors) in an eye (e.g., the retinal tissue of an eye). In some embodiments, the method includes treating symptoms associated with retinal cell loss (e.g., retinal pigment epithelial cells, retinal ganglion cells, and/or photoreceptors) in the eye (e.g., retinal degeneration, increased intraocular pressure, loss of and/or decrease in visual acuity, blurred vision, distorted vision, etc.).

In some embodiments, the method is a method of treating retinal degeneration. In some embodiments, the method is a method of inhibiting, reducing, and/or preventing retinal degeneration. In some embodiments, the method includes treating symptoms associated with retinal degeneration (e.g., increased intraocular pressure, loss of and/or decrease in visual acuity, blurred vision, distorted vision, etc.). In some embodiments, retinal degeneration comprises the loss and/or death of retinal pigment epithelial cells, retinal ganglion cells, photoreceptors, or a combination thereof. In some embodiments, retinal degeneration comprises the loss and/or death of retinal pigment epithelial cells. In some embodiments, retinal degeneration comprises the loss and/or death of retinal ganglion cells. In some embodiments, retinal degeneration comprises the loss and/or death of photoreceptors. In some embodiments, retinal degeneration comprises the loss and/or death a combination of retinal pigment epithelial cells, retinal ganglion cells, and/or photoreceptors.

In some embodiments, the method is a method of treating an ocular disease, disorder, or condition. In some embodiments, the method is a method of inhibiting, reducing, and/or preventing an ocular disease, disorder, or condition. In some embodiments, the method includes treating symptoms associated with an ocular disease, disorder, or condition (e.g., retinal degeneration, increased intraocular pressure, loss of and/or decrease in visual acuity, blurred vision, distorted vision, etc.). In some embodiments, the ocular disease, disorder, or condition is a chronic ocular disease, disorder, or condition. In some embodiments, the ocular disease, disorder, or condition comprises the loss and/or death of retinal pigment epithelial cells, retinal ganglion cells, photoreceptors, or a combination thereof. In some embodiments, the ocular disease, disorder, or condition comprises the loss and/or death of retinal pigment epithelial cells. In some embodiments, the ocular disease, disorder, or condition comprises the loss and/or death of retinal ganglion cells. In some embodiments, the ocular disease, disorder, or condition comprises the loss and/or death of photoreceptors. In some embodiments, the ocular disease, disorder, or condition comprises the loss and/or death a combination of retinal pigment epithelial cells, retinal ganglion cells, and/or photoreceptors.

In some embodiments, the method is a method of treating glaucoma. In some embodiments, the method is a method of inhibiting, reducing, and/or preventing glaucoma. In some embodiments, the method includes treating symptoms associated with glaucoma (e.g., retinal degeneration, increased intraocular pressure, loss of and/or decrease in visual acuity, blurred vision, distorted vision, etc.). In some embodiments, the ocular disease, disorder, or condition comprises the loss and/or death of retinal ganglion cells.

In some embodiments, the method is a method of treating macular degeneration. In some embodiments, the method is a method of inhibiting, reducing, and/or preventing macular degeneration. In some embodiments, the method includes treating symptoms associated with macular degeneration (e.g., retinal degeneration, increased intraocular pressure, loss of and/or decrease in visual acuity, blurred vision, distorted vision, etc.). In some embodiments, the macular degeneration is age-related macular degeneration, non-exudative age-related macular degeneration, or exudative age-related macular degeneration. In some embodiments, the macular degeneration is age-related macular degeneration. In some embodiments, the macular degeneration is non-exudative age-related macular degeneration. In some embodiments, the macular degeneration is exudative age-related macular degeneration. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors.

In some embodiments, the method is a method of treating retinal detachment. In some embodiments, the method is a method of inhibiting, reducing, and/or preventing retinal detachment. In some embodiments, the method includes treating symptoms associated retinal detachment (e.g., retinal degeneration, increased intraocular pressure, loss of and/or decrease in visual acuity, blurred vision, distorted vision, etc.). In some embodiments, the retinal detachment is chronic retinal detachment. In some embodiments, retinal detachment comprises the loss and/or death of retinal pigment epithelial cells, retinal ganglion cells, photoreceptors, or a combination thereof. In some embodiments, retinal detachment comprises the loss and/or death of retinal pigment epithelial cells. In some embodiments, retinal detachment comprises the loss and/or death of retinal ganglion cells. In some embodiments, retinal detachment comprises the loss and/or death of photoreceptors. In some embodiments, retinal detachment comprises the loss and/or death a combination of retinal pigment epithelial cells, retinal ganglion cells, and/or photoreceptors.

In some embodiments, the method is a method of treating a loss in visual acuity. In some embodiments, the method is a method of treating a loss in visual acuity. In some embodiments, a loss in visual acuity is characterized by and/or equivalent to a reduction in visual acuity (e.g., a reduction from 20/100 to 20/200, or a reduction from 20/40 to 20/50, etc.). In some embodiments, the method is a method of treating ocular hypertension (e.g., elevated intraocular pressure). In some embodiments, the method is a method of treating ocular hypertension (e.g., elevated intraocular pressure). In some embodiments, ocular hypertension is characterized by an intraocular pressures of 21 mm Hg or greater. In some embodiments, ocular hypertension includes by an intraocular pressures of 18 mm Hg or greater.

In some embodiments, the method is a method of treating a loss in visual function. In some embodiments, the method is a method of treating a loss in visual function. In some embodiments, a loss in visual function is characterized by and/or equivalent to a reduction in visual function assessments (e.g., visual acuity, field of vision, contrast sensitivity, binocular function, and/or light/dark adaptation).

In some embodiments, (y) is 30 to 360. In some embodiments, (y) is 90 to 180. In some embodiments, the peptide has a half-life in the vitreous humor of at least about (z) days. In some embodiments, (z) is 30 to 300.

In some embodiments, the method comprises using the vitreous humor as a depot to provide the peptide to retinal tissue in the eye, whereby the peptide is present in the vitreous humor for at (n) days after administration. In some embodiments, the peptide is provided to the retina from the vitreous humor for at least about (n) days after administration. In some embodiments, (n) is 30 to 365.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions.

In some embodiments, the peptide further comprises a modification. In some embodiments, modification comprises a modified amino acid. In some embodiments, the peptide comprises an amidated C-terminus. In some embodiments, the peptide has the structure of Formula III or a pharmaceutically-acceptable salt thereof.

In some embodiments, the peptide is detectable in the vitreous humor for at least about (z) days after administration. In some embodiments, the composition comprises the pharmaceutically-acceptable salt of the peptide. In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt. In some embodiments, the composition further comprises on or more excipients. In some embodiments, the composition further comprises a surfactant. In some embodiments, the surfactant is a non-ionic surfactant. In some embodiments, the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer. In some embodiments, the surfactant forms about 0.01% to about 20% weight/weight of the composition. In some embodiments, the surfactant forms about 0.05% to about 10% weight/weight of the composition. In some embodiments, the composition further comprises a tonicity adjusting agent, a buffering agent, or a combination thereof. In some embodiments, the composition is buffered at a pH of 2.5 to 7.5.

In some embodiments, the composition comprises 5 micrograms (ug) to 10,000 ug of the peptide. In some embodiments, the composition comprises at least 10 micrograms (ug), at least 25 ug, at least 50 ug, at least 100 ug, at least 150 ug, at least 200 ug, or at least 250 ug of the peptide. In some embodiments, the composition comprises 10 micrograms (ug), 25 ug, 50 ug, 100 ug, 150 ug, 200 ug, or 250 ug of the peptide. In some embodiments, the peptide is present at a concentration 0.1 milligrams per milliliter (mg/mL) to 10.0 mg/mL.

Provided herein are methods of treating an ocular disease, disorder, or condition, comprising: administering a first composition comprising a peptide comprising an amino acid sequence HHIYLGAVNYIY or a variant sequence thereof, or a pharmaceutically-acceptable salt of the peptide, to a vitreous humor of an eye; and administering at least a second composition comprising the peptide to the vitreous humor of the eye, wherein the at least second dose is administered no less than about 30 to 365 days after the first administration. In some embodiments, the at least second dose is administered no less than about 90 to about 180 days after the first administration.

Provided herein are methods of treating an ocular disease, disorder, or condition, comprising: administering a plurality of compositions to a vitreous humor of an eye, the plurality of compositions being administered no more frequently than once every about 30 to 365 days, and each of the plurality of compositions comprising a peptide comprising an amino acid sequence HHIYLGAVNYIY or variant sequence thereof, or a pharmaceutically-acceptable salt of the peptide. In some embodiments, the plurality of compositions being administered no more frequently than once every about 90 to 180 days. In some embodiments, the peptide has a half-life in the vitreous humor of (e.g., at least) about 30 to 300 days. In some embodiments, the peptide has a half-life in the vitreous humor of (e.g., at least) about 90 to 300 days.

In some embodiments, the method comprises using the vitreous humor as a depot to provide the peptide to retinal tissue in the eye, wherein the peptide is present in the vitreous humor for about 30 to 365 days after administration. In some embodiments, the peptide is provided to the retina from the vitreous humor for at least about 30 to 365 days after administration. In some embodiments, the peptide is present in the vitreous humor at about 90 to 365 days after administration. In some embodiments, the peptide is provided to the retina from the vitreous humor for at least about 30 days after administration. In some embodiments, the peptide is provided to the retina from the vitreous humor for at least about 90 days after administration.

Provided are methods of treating an ocular disease, disorder, or condition, comprising: administering a composition comprising a peptide comprising an amino acid sequence HHIYLGAVNYIY or a variant sequence thereof, or a pharmaceutically-acceptable salt of the peptide, to a vitreous humor of an eye, thereby providing the peptide to retinal tissue in the eye, wherein the peptide has a half-life in the vitreous humor of about 30 to 300 days. In some embodiments, the peptide has a half-life in the vitreous humor of at least about 90 to 300 days.

In some embodiments, the method comprises using the vitreous humor as a depot to provide a therapeutically-effective amount of the peptide to the retinal tissue (e.g., to reduce inflammation or reduce cell death or reduce a loss in visual acuity).

In some embodiments, the peptide is provided to the retina from the vitreous humor for at least about 14 to 365 days after administration. In some embodiments, the peptide is provided to the retina from the vitreous humor for at least about 30 days after administration. In some embodiments, the peptide is provided to the retina from the vitreous humor for at least about 90 days after administration.

In some embodiments, the ocular disease, disorder, or condition comprises inflammation in retinal tissue and/or a symptom thereof. In some embodiments, the ocular disease, disorder, or condition comprises photoreceptor cell death and/or a symptom thereof. In some embodiments, the ocular disease, disorder, or condition comprises retinal degeneration and/or a symptom thereof. In some embodiments, the ocular disease, disorder, or condition comprises a loss and/or decrease in visual acuity. In some embodiments, the ocular disease, disorder, or condition comprises macular degeneration and/or a symptom thereof. In some embodiments, the ocular disease, disorder, or condition comprises glaucoma and/or a symptom thereof.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions. In some embodiments, the peptide further comprises a modification. In some embodiments, the modification comprises a modified amino acid. In some embodiments, the peptide comprises an amidated C-terminus. In some embodiments, the peptide has the structure of Formula I or a pharmaceutically-acceptable salt thereof. In some embodiments, the peptide has the structure of Formula III or a pharmaceutically-acceptable salt thereof.

In some embodiments, the composition comprises the pharmaceutically-acceptable salt of the peptide. In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt. In some embodiments, the composition further comprises one or more excipients. In some embodiments, the composition further comprises a surfactant. In some embodiments, the surfactant is a non-ionic surfactant. In some embodiments, the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer. In some embodiments, the surfactant forms about 0.01% to about 20% weight/weight of the composition. In some embodiments, the surfactant forms about 0.05% to about 10% weight/weight of the composition.

In some embodiments, the composition further comprises a tonicity adjusting agent, a buffering agent, or a combination thereof. In some embodiments, the composition is buffered at a pH of 2.5 to 7.5. In some embodiments, the composition comprises 5 micrograms (ug) to 10,000 ug of the peptide. In some embodiments, the composition comprises at least 10 micrograms (ug), at least 25 ug, at least 50 ug, at least 100 ug, at least 150 ug, at least 200 ug, or at least 250 ug of the peptide. In some embodiments, the composition comprises 10 micrograms (ug), 25 ug, 50 ug, 100 ug, 150 ug, 200 ug, or 250 ug of the peptide. In some embodiments, the peptide is present at a concentration 0.1 milligrams per milliliter (mg/mL) to 10.0 mg/mL.

Further provided herein are methods of treating an ocular disease, disorder, or condition (e.g., a chronic disorder) or symptoms thereof (e.g., a reduction in visual acuity), comprising chronically administering a composition comprising a peptide comprising an amino acid sequence HHIYLGAVNYIY or a variant sequence thereof, or a pharmaceutically-acceptable salt of the peptide, to a vitreous humor of an eye, wherein the composition is administered no greater than 5 times a year. In some embodiments, the composition is administered no greater than 4 times a year. In some embodiments, the composition is administered no greater than than 3 times a year. In some embodiments, the composition is administered no greater than 2 times a year. In some embodiments, the composition is administered no greater than once a year.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. shows data demonstrating extended protection against retinal cell death by a Fas-inhibitor.

### DETAILED DESCRIPTION

Retinal degeneration is a complex, multifactorial condition where cell death (e.g., cell apoptosis), glial cell activation, and inflammation in or surrounding retinal tissue have been linked to the death of cells within the retina (e.g., retinal pigment epithelial (RPE) cells, retinal ganglion cells (RGCs), and/or photoreceptors (PRs)). Fas-mediated inflammation can directly or indirectly lead to retinal degeneration and can be associated with the symptoms resulting from retinal degeneration. The Fas-mediated inflammation signaling pathway is generally initiated by an interaction between the membrane-bound Fas ligand (FasL—a type II transmembrane protein of the TNF family) and Fas receptor, thereby leading to the activation of pro-inflammatory signaling (e.g., cytokine signaling, interleukin signaling, caspase action, etc.) and/or cell death signaling (e.g., apoptotic signaling, necrotic signaling, etc.) pathways. The methods and compositions provided herein are useful in inhibiting, preventing, and/or reducing Fas-mediated inflammation in the eye of an individual. Notably, the methods and compositions provided herein are useful in inhibiting, preventing, and/or reducing Fas-mediated inflammation over an extended period of time in the eye, thereby reducing the frequency of administration as compared with known ocular therapies. Accordingly, such methods and compositions provide advantages over traditional ocular therapies marked by short pharmacokinetic and pharmacodynamic profiles that require repeated injections to the eye to achieve a therapeutic benefit and/or an effective dosing program.

### Fas Inhibiting Peptides

Provided herein are Fas inhibitors useful in treating, inhibiting, preventing, and/or reducing Fas-mediated inflammation. In some embodiments, inhibiting, preventing, and/or reducing Fas-mediated inflammation allows for the treatment and/or prevention of retinal degeneration. Accordingly, in some embodiments, the Fas inhibitors are also useful in treating and/or preventing retinal degeneration and/or the symptoms associated with retinal degeneration. In some embodiments, inhibiting, preventing, and/or reducing Fas-mediated inflammation further treats, inhibits, reduces, and/or prevents retinal cell loss (e.g., retinal epithelial (RPE) cells, retinal ganglion cells (RGCs), and/or photoreceptor (PR)). cells). In some embodiments, the Fas inhibitors described herein are useful for treating, inhibiting, reducing, and/or preventing retinal cell loss and/or the symptoms associated with retinal cell loss. In further instances, because the Fas inhibitors described herein are useful in the manner described above, the Fas inhibitors are useful in method of treating ocular diseases and disorders. In further embodiments, the Fas inhibitors described herein are useful in treating an ocular disease or disorder.

In an embodiment, the Fas inhibitors described herein encompass Met-derived peptides and/or fragments thereof. In some embodiments, the Met protein, also called c-Met or hepatocyte growth factor receptor (HGF receptor), is encoded by the Met gene (NCBI Gene ID 4233, Location: NC_000007.14 (116672196 . . . 116798386), UniProtKB—P0858). In some embodiments, Met is comprised of two major subunits: the α and β subunits. Met and fragments of Met, including the extracellular domain of Met and its α subunit, have been shown to bind to Fas and prevent cells from undergoing apoptosis. In some embodiments, the Fas inhibitor comprises a Met-derived peptide and/or fragment thereof. In some embodiments, the Fas inhibitors described herein comprises a Met-derived compound comprising the amino acid acids HHIYLGAVNYIY (His-His-lle-Tyr-Leu-Gly-Ala-Val-Asn-Tyr-lle-Tyr) (e.g., SEQ ID NOs: 1-8). In some embodiments, the peptide comprises the amino acid sequence HHIYLGAVNYIY or a variant sequence thereof.

As used herein, a peptide includes and/or refers to any of various natural or synthetic compounds containing two or more amino acids joined by a peptide bond that link the carboxyl group of one amino acid to the amino group of another. As also used herein, amino acid refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission.

In some embodiments, the Fas inhibitor peptides (e.g., a peptide comprising the amino acid sequence HHIYLGAVNYIY) comprises one or more naturally occurring amino acids. In some embodiments, the Fas inhibitor peptides (e.g., a peptide comprising the amino acid sequence HHIYLGAVNYIY) consists of naturally occurring amino acids. As used herein, naturally occurring amino acids include and/or refer to amino acids which are generally found in nature and are not manipulated by man. In some embodiments, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the Fas inhibitor comprises a variant sequence of the peptide (e.g., a peptide comprising the amino acid sequence HHIYLGAVNYIY). In some embodiments, amino acid substitutions can be made in the sequence of any of the polypeptides described herein, without necessarily decreasing or ablating its activity. Accordingly, in some embodiments, the variant sequence comprises one or more amino acid substitutions. In some embodiments, the variant sequence comprises one amino acid substitution. In some embodiments, the variant sequence comprises two amino acid substitutions. In some embodiments, the variant sequence comprises three amino acid substitutions. In some embodiments, substitutions include conservative substitutions (e.g., substitutions with amino acids of comparable chemical characteristics). In some embodiments, a non-polar amino acid can be substituted and replaced with another non-polar amino acid, wherein non-polar amino acids include alanine, leucine, isoleucine, valine, glycine, proline, phenylalanine, tryptophan and methionine. In some embodiments, a neutrally charged polar amino acids can be substituted and replaced with another neutrally charged polar amino acid, wherein neutrally charged polar amino acids include serine, threonine, cysteine, tyrosine, asparagine, and glutamine. In some embodiments, a positively charged amino acid can be substituted and replaced with another positively charged amino acid, wherein positively charged amino acids include arginine, lysine and histidine. In some embodiments, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

In some embodiments, the Fas inhibitor peptides (e.g., a peptide comprising the amino acid sequence HHIYLGAVNYIY) comprises one or more non-natural amino acids. In some embodiments, the Fas inhibitor peptides (e.g., a peptide comprising the amino acid sequence HHIYLGAVNYIY) consists of non-natural amino acids. As used herein, non-natural amino acids and/or unnatural amino acids include and/or refer to amino acid structures that cannot be generated biosynthetically in any organism using unmodified or modified genes from any organism. In some embodiments, non-natural amino acids and/or unnatural amino acids further include and/or refer to an amino acid residues that are not present in the naturally occurring (wild-type) Met protein sequence. For example, these include, but are not limited to, modified amino acids and/or amino acid analogues that are not one of the 20 naturally occurring amino acids (e.g., non-natural side chain variant sequence amino acids), D-amino acids, homo amino acids, beta-homo amino acids, N-methyl amino acids, alpha-methyl amino acids, or. By way of further example, non-natural amino acids also include 4-Benzoylphenylalanine (Bpa), Aminobenzoic Acid (Abz), Aminobutyric Acid (Abu), Aminohexanoic Acid (Ahx), Aminoisobutyric Acid (Aib), Citrulline (Cit), Diaminobutyric Acid (Dab), Diaminopropanoic Acid (Dap), Diaminopropionic Acid (Dap), Gamma-Carboxyglutamic Acid (Gla), Homoalanine (Hala), Homoarginine (Harg), Homoasparagine (Hasn), Homoaspartic Acid (Hasp), Homocysteine (Hcys), Homoglutamic Acid (Hglu), Homoglutamine (Hgln), Homoisoleucine (Hile), Homoleucine (Hleu), Homomethionine (Hmet), Homophenylalanine (Hphe), Homoserine (Hser), Homotyrosine (Htyr), Homovaline (Hval), Hydroxyproline (Hyp), Isonipecotic Acid (Inp), N aphthylalanine (Nal), Nipecotic Acid (Nip), Norleucine (Nle), Norvaline (Nva), Octahydroindole-2-carboxylic Acid (Oic), Penicillamine (Pen), Phenylglycine (Phg), Pyroglutamic Acid (Pyr), Sarcosine (Sar), tButylglycine (Tle), and Tetrahydro-isoquinoline-3-carboxylic Acid (Tic). Such non-natural amino acid residues can be introduced by substitution of naturally occurring amino acids, and/or by insertion of non-natural amino acids into the naturally occurring (wild-type) Met protein sequence. A non-natural amino acid residue also can be incorporated such that a desired functionality is imparted to the apelin molecule, for example, the ability to link a functional moiety (e.g., PEG).

In some embodiments, a variant sequence comprises one or more amino acid deletions. In some embodiments, the variant sequence comprises one amino acid deletion. In some embodiments, the variant sequence comprises two amino acid deletions. In some embodiments, the variant sequence comprises three amino acid deletions. In some embodiments, the variant sequence comprises four amino acid deletions. In some embodiments, the variant sequence comprises one or more additional amino acids. In some embodiments, the additional amino acids are additional amino acids from the Met-12 sequence. In some embodiments, the variant sequence comprises a substitution and a deletion. In some embodiments, the variant sequence comprises a substitution and one or more additional amino acids. In some embodiments, the substitution comprises a natural amino acid or a non-natural amino acid. In some embodiments, the variant sequence is a retro inverso amino acid sequence.

Functionality of variant sequences of the peptide (e.g., a variant sequence of the amino acid sequence HHIYLGAVNYIY) can be determined by an in vitro assay. For example, in some embodiments, the variant sequence competes for binding to a Fas receptor (FasR) with Fas ligand (FasL). In some embodiments, the variant sequence inhibits, reduces, or prevents caspase 8 activation in cells treated with FasL (e.g., as measured by commercially available luminescent tetrapeptide cleavage assay kit (Promega, Madison, WI)). In some embodiments, the variant sequence inhibits, reduces, or prevents cell death of cells treated with FasL. By way of further example, in some embodiments, the variant sequence competes for binding to a Fas receptor (FasR) with a Fas-activating antibody (e.g., Fas-agonistic Jo2 monoclonal antibody (BD Biosciences, San Jose, CA)). In some embodiments, the variant sequence inhibits, reduces, or prevents caspase 8 activation in cells treated with a Fas-activating antibody (e.g., as measured by commercially available luminescent tetrapeptide cleavage assay kit (Promega, Madison, WI)). In some embodiments, the variant sequence inhibits, reduces, or prevents cell death of cells treated with a Fas-activating antibody. Accordingly, in some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with Fas ligand (FasL). In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents caspase 8 activation in cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence inhibits, reduces, or prevents cell death of cells treated with FasL. In some embodiments, the Fas inhibitor comprises a variant sequence (e.g., any one of the variant sequences described herein) of the peptide (e.g., a peptide comprising the amino acid sequence HHIYLGAVNYIY), wherein the variant sequence competes for binding to a Fas receptor (FasR) with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents caspase 8 activation in cells treated with a Fas-activating antibody. In some embodiments, the Fas inhibitor comprises a variant sequence (e.g., any one of the variant sequences described herein) of the peptide comprising the amino acid sequence HHIYLGAVNYIY, wherein the variant sequence inhibits, reduces, or prevents cell death of cells treated with a Fas-activating antibody.

The peptide or a variant sequence thereof can further comprise one or more modifications. In some embodiments, the peptide (e.g., a comprising the amino acid sequence HHIYLGAVNYIY or a variant sequence thereof) comprises a modification. In some embodiments, the peptide is a modified peptide. As used herein, a modification or a modified peptide includes and/or refers to a modification of one or more amino acids in the peptide. In some embodiments, modifications species of stereoisomers. All stereoisomers of the above compounds are contemplated, either in admixture or in pure or substantially pure form. The compounds can have asymmetric centers at any of the atoms. Consequently, the peptide compounds or components thereof can exist in enantiomeric or diastereomeric forms or in mixtures thereof. The present invention contemplates the use of any racemates (i.e., mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R, S or d, D, 1,L ord, 1, D,L. Compounds comprising amino acid residues include residues of D-amino acids, L-amino acids, or racemic derivatives of amino acids. Compounds comprising sugar residues include residues of D-sugars, L-sugars, or racemic derivatives of sugars. Non-limiting examples of modifications are phosphorylation, glycosylation, ubiquitination, nitrosylation, methylation, acetylation, amidation, or lipidation. Modification can be introduced at the C-terminus of the peptide, the N-terminus of the peptide, or at any place in-between. Thus, a modification or a modified peptide includes and/or refers to modifications of the free amino- and/or carboxyl-terminal (N-terminus and C-terminus, respectively). In some embodiments, N-terminal modifications include but are not limited to acetylation, formylation, pyroglutamylation, carbamide addition, lipidation, sulfonamidation, and alkylamination. In some embodiments, C-terminal modifications include but are not limited to amidation, esterification, and incorporation of an aldehyde group. In some embodiments, the modification comprises amidation. In some embodiments, the amidation is at the c-terminus. In some embodiments, the modification comprises a retro inverso peptide (e.g., YIYNVAGLYIHH)

Accordingly, in an embodiment, provided herein are peptides comprising the sequence
(a)-HHIYLGAVNYIY-(b) or (a)-YIYNVAGLYIHH-(b), or a variant sequence thereof, wherein:
   (a) is —H, —OH, —NH₂, G¹(CH₂)ₙ—, R¹CONH—, or R²O—;
   (b) is —H, —CH₂OH, —CH₂OR², —CHO, —CO₂R², —CONH₂, —CONHR₂, —CON(R³)₂, —CONH(CH₂)_{y}NR⁽³⁾₂, —(CH₂)ₙ-G¹, —COCH₂-G¹, —CONHCH₂-G¹, —(CH₂)ₙNH₂, —(CH₂)ₙNHR², —(CH₂)ₙN(R³)₂, NH-Glu-His —OH, NH-Glu-His —NH₂, -Ala-His —NH₂, -Gly-His —NH₂, —NH-Glu-His —OH, —NH-Glu-His —NH₂, -Ala-His —NH₂, -Gly-His —NH₂, —NH-[D]Glu-[D]-His —OH, —NH-[D]Glu-[D]-His —NH₂, -[D]Ala-[D]-His —NH₂, -Gly[D]-His-NH₂, or —CONH(CH₂)ₙ-G²; G¹, at each occurrence, is independently-H, —C(═O)NH₂, —C(═O)NHR², —C(═O)N(R³)₂, C(—O)OR², or —C(═O)R¹; G² at each occurrence is a heterocyclic ring of 4-7 members comprising at least one tertiary amine functionality NR² within the ring, or a carbocyclic ring of 3-7 members substituted with —N(R³)₂; R¹, at each occurrence, is independently H, C₁₋₆ alkyl, —(CH₂)ₓ(OCH₂CH₂)ₘOR⁵, C₁₋₆-alkoxy or L;
   R², at each occurrence, is independently C₁₋₆ alkyl, C₂₋₆ alkyl substituted with OR⁵ or NR⁵², —(CH₂)ₓ(OCH₂CH₂)ₘOR⁵ or L;
   L, at each occurrence, is a multivalent polyethylene glycol derivative with 2-4 termini, each of which may be independently capped with H, R⁵;
   R³, at each occurrence, is independently C₁₋₆ alkyl, C₂₋₆ alkyl substituted with OR⁵ or N(R⁵)₂, —(CH₂)ₓ(OCH₂CH₂)ₘOR⁵;
   or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;
   R⁴, at each occurrence, is independently C₁₋₆ alkyl, C₁₋₆ acyl, or —OPO₃(R⁵)₂;
   R⁵, at each occurrence, is independently H or C₁₋₆ alkyl;
   R⁶, at each occurrence, is H, C₁₋₆ alkyl, C₂₋₆ hydroxyalkyl, C₁₋₆alkoxy-, C₁₋₆ alkyl, or C₁₋₆ acyl;
   m=1-100;
   n=0-3;
   x=0-6; and
   y=2-4,
   wherein at most one of R¹ and R² is L.

In another embodiment, provided herein are peptides comprising the structure of Formula I or Formula II, or a pharmaceutically-acceptable salt thereof.

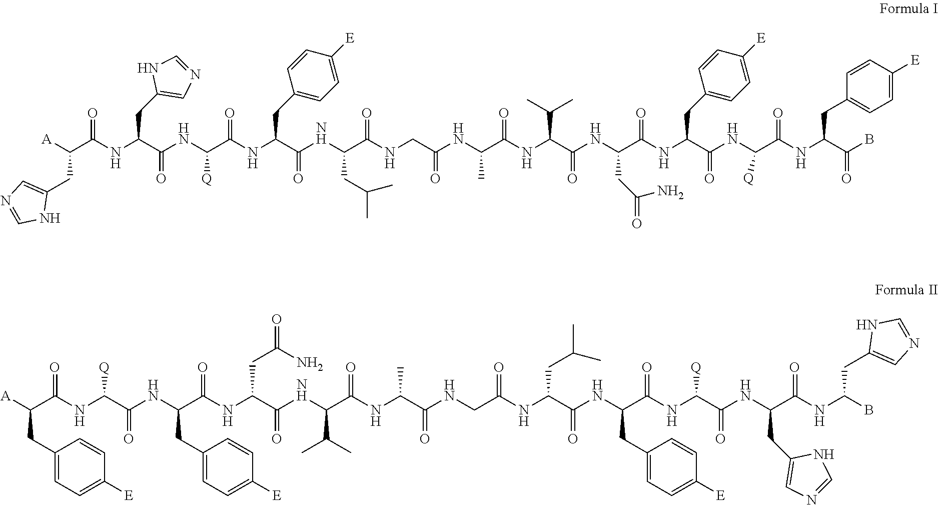

wherein:
A is H—, —OH, —NH₂, G¹(CH₂)ₙ—, R¹CONH—, or R²O—;
B is —H, CH₂OH, CH₂OR², —CHO, —CO₂R², —CONH₂, —CONHR₂, —CON(R³)₂, —CONH(CH₂)_{y}N(R³)₂, —(CH₂)ₙ-G¹, —COCH₂-G¹, —CONHCH₂-G¹, —(CH₂)ₙNH₂, —(CH₂)ₙNHR², —(CH₂)ₙN(R³)₂ NH-Glu-His —OH, NH-Glu-His —NH₂, -Ala-His —NH₂, -Gly-His —NH₂, NH-Glu—His —OH, NH-Glu-His —NH₂, -Ala-His —NH₂, -Gly-His —NH₂, NH-[D]Glu-[D]-His —OH, NH-[D]Glu-[D]-His —NH₂, -[D]Ala-[D]-His —NH₂, -Gly[D]-His —NH₂, or CONH(CH₂)ₙ-G²;
E, at each occurrence, is independently-H, —OH, —OR⁴, SH, —SR⁴, or halogen;
G¹, at each occurrence, is independently-H, —C(═O)NH₂, —C(═O)NHR², —C(═O)N(R³)₂, C(═O)OR², or —C(═O)R¹;
G² at each occurrence is a heteroalicyclic ring of 4-7 members comprising at least one tertiary amine functionality NR² within the ring, or a carbocyclic ring of 3-7 members substituted with N(R³)₂;
Q, at each occurrence, is independently, 1-propyl, 2-propyl, 2-methyl-prop-2-yl, C₃₋₆-cycloalkyl, C₄₋₆-cycloalkenyl, tetrahydrofuran-2-yl, tetrahydrofuran-3-yl, tetrahydrothienyl-2-yl, tetrahydrothienyl-3-yl, tetrahydropyran-2-yl, tetrahydropyran-3-yl, tetrahydropyran-4-yltetrahydrothiopyran-2-yl, tetrahydrothiopyran-3-yl, tetrahydrothiopyran-4-yl or 1-CH(OR⁵) cH₃;
R¹, at each occurrence, is independently H, C₁₋₆ alkyl, —(CH₂)ₓ(OCH₂CH₂)ₘOR⁵, C₁₋₆ alkoxy or L;
R², at each occurrence, is independently C₁₋₆ alkyl, C₂₋₆ alkyl substituted with OR⁵ or N(R⁵)₂, —(CH₂)ₓ(OCH₂CH₂)ₘOR⁵ or L;
L, at each occurrence, is a multivalent polyethylene glycol derivative with 2-4 termini, each of which may be independently capped with H, R⁵ or another molecule of the peptide of Formula I or II;
R³, at each occurrence, is independently C₁₋₆-alkyl, C₂₋₆-alkyl substituted with OR⁵ or N(R⁵)₂, —(CH₂)ₓ(OCH₂CH₂)ₘOR⁵;
or two R³s, taken together with the N atom to which they are attached, may form a monocyclic ring of 4-8 members or a fused, bridged or spiro bicyclic ring of 6-10 members, which can include up to two groups within the ring chosen independently from —O—, —(C═O)—, NR⁶, S, SO, or SO₂;
R⁴, at each occurrence, is independently C₁₋₆ alkyl, C₁₋₆ acyl, or —OPO₃(R⁵)₂;
R⁵, at each occurrence, is independently H or C₁₋₆ alkyl;
R⁶, at each occurrence, is H, C₁₋₆ alkyl, C₂₋₆ hydroxyalkyl, C₁₋₆alkoxy-, C₁₋₆ alkyl, or C₁₋₆ acyl;
m=1-100;
n=0-3;
x=0-6; and
y=2-4,
wherein at most one of R¹ and R² is L.

In some embodiments, provided is a peptide having the structure of Formula III or a pharmaceutically-acceptable salt thereof:

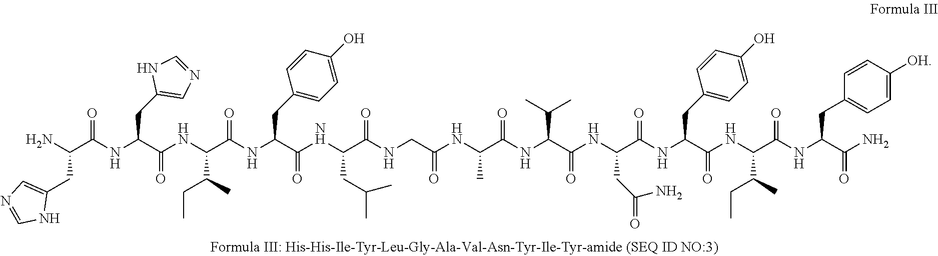 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]

In some embodiments, provided is a peptide having the structure of Formula IV or a pharmaceutically-acceptable salt thereof:

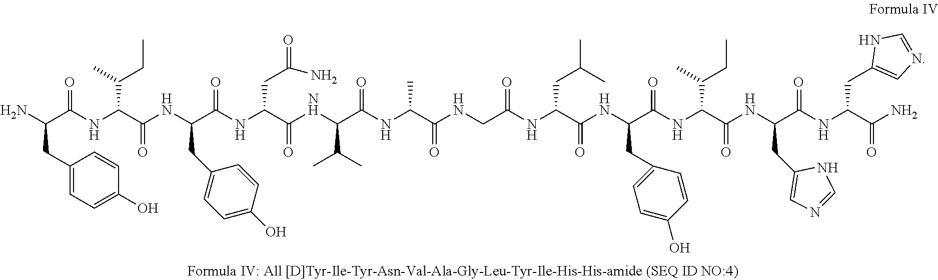 [SMILES: CC[C@@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@H](C)CC)C(C)C]

In some embodiments, provided is a peptide having the structure of Formula V or a pharmaceutically-acceptable salt thereof:

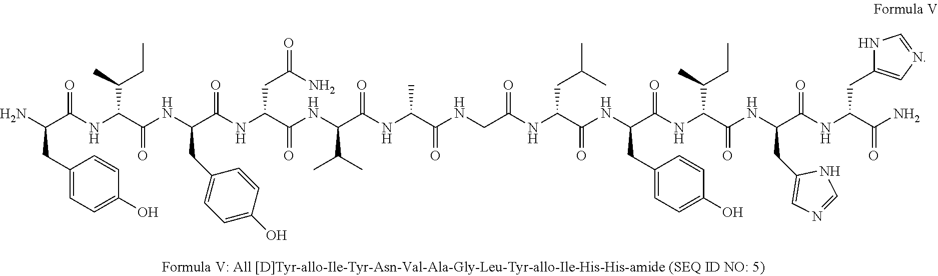 [SMILES: CC[C@H](C)[C@@H](NC(=O)[C@H](N)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@@H](C)CC)C(C)C]

In some embodiments, provided is a peptide having the structure of Formula VI or a pharmaceutically-acceptable salt thereof:

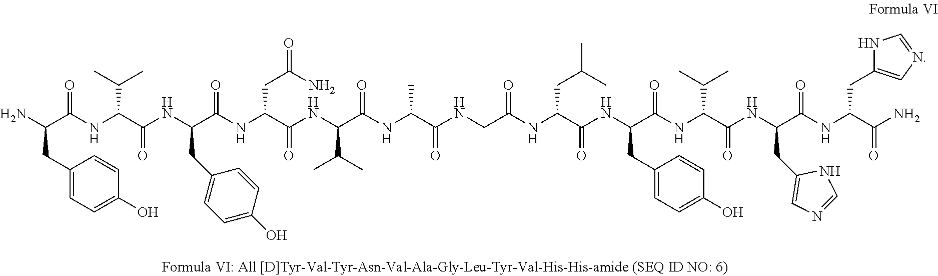 [SMILES: CC(C)C[C@@H](NC(=O)CNC(=O)[C@@H](C)NC(=O)[C@H](NC(=O)[C@@H](CC(N)=O)NC(=O)[C@@H](Cc1ccc(O)cc1)NC(=O)[C@H](NC(=O)[C@H](N)Cc1ccc(O)cc1)C(C)C)C(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)C(C)C]

In some embodiments, provided is a peptide having the structure of Formula VII or a pharmaceutically-acceptable salt thereof:

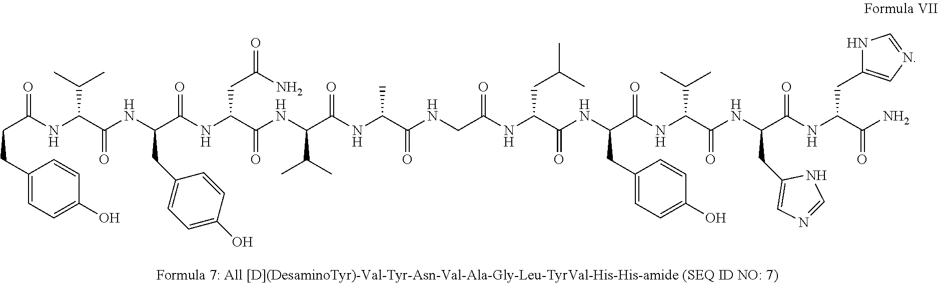

In some embodiments, provided is a peptide having the structure of Formula VIII or a pharmaceutically-acceptable salt thereof:

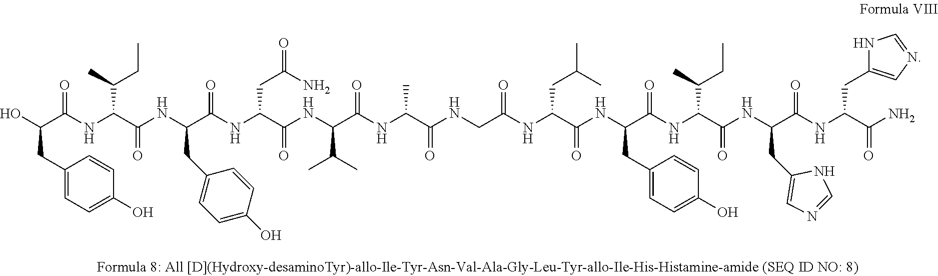 [SMILES: CC[C@H](C)[C@@H](NC(=O)[C@H](O)Cc1ccc(O)cc1)C(=O)N[C@H](Cc1ccc(O)cc1)C(=O)N[C@H](CC(N)=O)C(=O)N[C@@H](C(=O)N[C@H](C)C(=O)NCC(=O)N[C@H](CC(C)C)C(=O)N[C@H](Cc1ccc(O)cc1)C(=O)N[C@@H](C(=O)N[C@H](Cc1cnc[nH]1)C(=O)N[C@H](Cc1cnc[nH]1)C(N)=O)[C@@H](C)CC)C(C)C]

In some embodiments, provided is a peptide having the structure of Formula IX or a pharmaceutically-acceptable salt thereof:

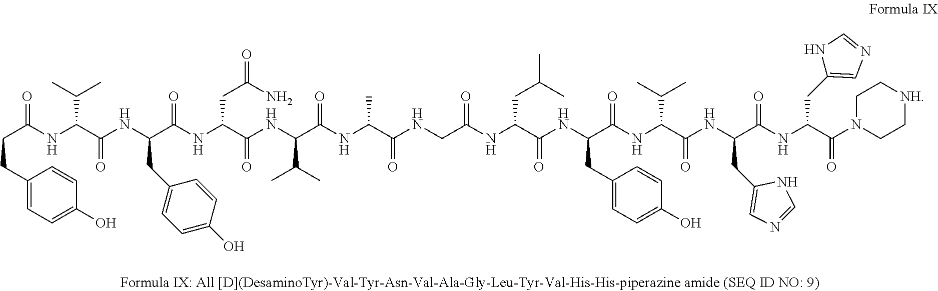

### Salts of Fas Inhibiting Peptides

Further provided herein are salts of the peptide for inhibiting Fas-mediated inflammation in the eye and for use in the methods described here. As used herein, salt is generally synonymous with pharmaceutically-acceptable salts, and/or includes or refers to pharmaceutically-acceptable salts. Examples of pharmaceutically-acceptable salts are salts with organic or inorganic acids such as (but not limited to) include acetic acid, aspartic acid, benzenesulfonic acid, benzoic acid, butyric acid, citric acid, fumaric acid, hydrochloric acid, hydrobromic acid, lactic acid, maleic acid, malonic acid, methanesulfonic acid, 4-methylbenzenesulfonic acid, nicotinic acid, phosphoric acid, succinic acid, sulfuric acid, or tartaric acid, prepared using methods well known in the art. In some embodiments, the salt is a hydrochloride salt.

In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, and polyamine resins.

Salts and pharmaceutically acceptable salts are described in in J. Pharmaceutical Sciences, 66: 1-19 (1977), the contents of which are incorporated by reference herein.

In some embodiments, the salt is an acetate salt. In some embodiments, the acetate salt is a poly-acetate salt. In some embodiments, the poly-acetate salt is a tri-acetate salt.

### Pharmaceutical Compositions

In an embodiment, further provided are pharmaceutical compositions (also referred to as compositions) comprising the Fas inhibiting peptides. In some embodiments, the pharmaceutical compositions described herein comprise the peptide (e.g., a peptide comprising the amino acid sequence HHIYLGAVNYIY) or a pharmaceutically-acceptable salt thereof. In some embodiments, the pharmaceutical compositions described herein comprise the peptide comprising the amino acid sequence HHIYLGAVNYIY or variant sequence thereof, or a pharmaceutically-acceptable salt of the peptide. In some embodiments, the pharmaceutical compositions described herein comprise the peptide having the structure of any one of Formulas I-IX or a pharmaceutically-acceptable salt thereof. In some embodiments, the pharmaceutical compositions described herein comprise the peptide having the structure of Formula I or a pharmaceutically-acceptable salt thereof. In some embodiments, the pharmaceutical compositions described herein comprise the peptide having the structure of Formula III or a pharmaceutically-acceptable salt thereof.

The pharmaceutical composition can comprise one or more excipients. As used herein, an excipient includes and/or refers to any pharmaceutically acceptable additive, carrier, diluent, adjuvant, or other ingredient, other than the active pharmaceutical ingredient (API), which is typically included for formulation and/or administration to a patient. A pharmaceutical composition can comprise a single pharmaceutical formulation (e.g., extended release, immediate release, delayed release, nanoparticulate, etc.) or multiple formulations (e.g., immediate release and delayed release, nanoparticulate and nonnanoparticulate, etc.). An excipient further includes and/or refers to an agent that may be added to a formulation to provide a desired consistency (e.g., altering the bulk properties), to improve stability, and/or to adjust osmolality. Examples of commonly used excipients include, but are not limited to, sugars, polyols, amino acids, surfactants, and polymers. In some embodiments, a non-ionic excipient or a non-ionizable excipient, as used herein, includes and/or refers to an agent having no net charge.

In some embodiments, the non-ionic excipient has no net charge under certain formulation conditions, such as pH. Examples of non-ionic excipients include, but are not limited to, sugars (e.g., sucrose), sugar alcohols (e.g., mannitol), and non-ionic surfactants (e.g., polysorbate 80).

In some embodiments, the compositions comprise excipients that are suitable for ocular application. Suitable excipients and include, but are not limited to, tonicity agents, preservatives, chelating agents, buffering agents, surfactants, cosolvents and antioxidants. Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1, and the like. Suitable chelating agents include sodium edetate and the like. Suitable buffering agents include phosphates, borates, citrates, acetates, tromethamine, and the like. Suitable surfactants include ionic and nonionic surfactants. In some embodiments, the one or more excipients comprises nonionic surfactants, such as polysorbates, polyethoxylated castor oil derivatives, polyethoxylated fatty acids, polyethoxylated alcohols, polyoxyethylene-polyoxypropylene block copolymers (Poloxamer), and oxyethylated tertiary octylphenol formaldehyde polymer (Tyloxapol). Other suitable surfactants may also be included. Suitable antioxidants include sulfites, thiosulfate, ascorbates, BHA, BHT, tocopherols, and the like.

In some embodiments, the composition comprises a non-ionic surfactant. In some embodiments, the composition comprises a polysorbate, a polyethoxylated castor oil derivative, a polyethoxylated fatty acid, a polyethoxylated alcohol, a polyoxyethylene-polyoxypropylene block copolymer (Poloxamer), or an oxyethylated tertiary octylphenol formaldehyde polymer (Tyloxapol). In some embodiments, the composition comprises a polysorbate. In some embodiments, the composition comprises a polyethoxylated castor oil derivative. In some embodiments, the composition comprises a polyethoxylated fatty acid. In some embodiments, the composition comprises a polyethoxylated alcohol. In some embodiments, the composition comprises a polyoxyethylene-polyoxypropylene block copolymer (Poloxamer). In some embodiments, the composition comprises an oxyethylated tertiary octylphenol formaldehyde polymer (Tyloxapol). In some embodiments, the surfactant makes up 0.05%-20% weight per weight (w/w) of the composition. In some embodiments, the non-ionic surfactant is about 0.05% w/w of the composition to about 20% w/w of the composition. In some embodiments, the non-ionic surfactant is at least about 0.05% w/w of the composition. In some embodiments, the non-ionic surfactant is at most about 20% w/w of the composition. In some embodiments, the non-ionic surfactant is about 0.05% w/w of the composition to about 0.1% w/w of the composition, about 0.05% w/w of the composition to about 0.5% w/w of the composition, about 0.05% w/w of the composition to about 1% w/w of the composition, about 0.05% w/w of the composition to about 2% w/w of the composition, about 0.05% w/w of the composition to about 5% w/w of the composition, about 0.05% w/w of the composition to about 10% w/w of the composition, about 0.05% w/w of the composition to about 20% w/w of the composition, about 0.1% w/w of the composition to about 0.5% w/w of the composition, about 0.1% w/w of the composition to about 1% w/w of the composition, about 0.1% w/w of the composition to about 2% w/w of the composition, about 0.1% w/w of the composition to about 5% w/w of the composition, about 0.1% w/w of the composition to about 10% w/w of the composition, about 0.1% w/w of the composition to about 20% w/w of the composition, about 0.5% w/w of the composition to about 1% w/w of the composition, about 0.5% w/w of the composition to about 2% w/w of the composition, about 0.5% w/w of the composition to about 5% w/w of the composition, about 0.5% w/w of the composition to about 10% w/w of the composition, about 0.5% w/w of the composition to about 20% w/w of the composition, about 1% w/w of the composition to about 2% w/w of the composition, about 1% w/w of the composition to about 5% w/w of the composition, about 1% w/w of the composition to about 10% w/w of the composition, about 1% w/w of the composition to about 20% w/w of the composition, about 2% w/w of the composition to about 5% w/w of the composition, about 2% w/w of the composition to about 10% w/w of the composition, about 2% w/w of the composition to about 20% w/w of the composition, about 5% w/w of the composition to about 10% w/w of the composition, about 5% w/w of the composition to about 20% w/w of the composition, or about 10% w/w of the composition to about 20% w/w of the composition. In some embodiments, the non-ionic surfactant is about 0.05% w/w of the composition, about 0.1% w/w of the composition, about 0.5% w/w of the composition, about 1% w/w of the composition, about 2% w/w of the composition, about 5% w/w of the composition, about 10% w/w of the composition, or about 20% w/w of the composition.

In some embodiments, the non-ionic surfactant is about 0.05% w/w of the composition to about 2% w/w of the composition. In some embodiments, the non-ionic surfactant is at least about 0.05% w/w of the composition. In some embodiments, the non-ionic surfactant is at most about 2% w/w of the composition. In some embodiments, the non-ionic surfactant is about 0.05% w/w of the composition to about 0.1% w/w of the composition, about 0.05% w/w of the composition to about 0.1% w/w of the composition, about 0.05% w/w of the composition to about 0.2% w/w of the composition, about 0.05% w/w of the composition to about 0.3% w/w of the composition, about 0.05% w/w of the composition to about 0.4% w/w of the composition, about 0.05% w/w of the composition to about 0.5% w/w of the composition, about 0.05% w/w of the composition to about 0.6% w/w of the composition, about 0.05% w/w of the composition to about 1% w/w of the composition, about 0.05% w/w of the composition to about 1.5% w/w of the composition, about 0.05% w/w of the composition to about 2% w/w of the composition, about 0.1% w/w of the composition to about 0.1% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.2% w/w of the composition to about 0.3% w/w of the composition, about 0.2% w/w of the composition to about 0.4% w/w of the composition, about 0.2% w/w of the composition to about 0.5% w/w of the composition, about 0.2% w/w of the composition to about 0.6% w/w of the composition, about 0.2% w/w of the composition to about 1% w/w of the composition, about 0.2% w/w of the composition to about 1.5% w/w of the composition, about 0.2% w/w of the composition to about 2% w/w of the composition, about 0.3% w/w of the composition to about 0.4% w/w of the composition, about 0.3% w/w of the composition to about 0.5% w/w of the composition, about 0.3% w/w of the composition to about 0.6% w/w of the composition, about 0.3% w/w of the composition to about 1% w/w of the composition, about 0.3% w/w of the composition to about 1.5% w/w of the composition, about 0.3% w/w of the composition to about 2% w/w of the composition, about 0.4% w/w of the composition to about 0.5% w/w of the composition, about 0.4% w/w of the composition to about 0.6% w/w of the composition, about 0.4% w/w of the composition to about 1% w/w of the composition, about 0.4% w/w of the composition to about 1.5% w/w of the composition, about 0.4% w/w of the composition to about 2% w/w of the composition, about 0.5% w/w of the composition to about 0.6% w/w of the composition, about 0.5% w/w of the composition to about 1% w/w of the composition, about 0.5% w/w of the composition to about 1.5% w/w of the composition, about 0.5% w/w of the composition to about 2% w/w of the composition, about 0.6% w/w of the composition to about 1% w/w of the composition, about 0.6% w/w of the composition to about 1.5% w/w of the composition, about 0.6% w/w of the composition to about 2% w/w of the composition, about 1% w/w of the composition to about 1.5% w/w of the composition, about 1% w/w of the composition to about 2% w/w of the composition, or about 1.5% w/w of the composition to about 2% w/w of the composition. In some embodiments, the non-ionic surfactant is about 0.05% w/w of the composition, about 0.1% w/w of the composition, about 0.1% w/w of the composition, about 0.2% w/w of the composition, about 0.3% w/w of the composition, about 0.4% w/w of the composition, about 0.5% w/w of the composition, about 0.6% w/w of the composition, about 1% w/w of the composition, about 1.5% w/w of the composition, or about 2% w/w of the composition.

In some embodiments, the non-ionic surfactant comprises Polysorbate 20, Poloxamer 407, Tyloxapol, or cremophor. In some embodiments, the non-ionic surfactant is Polysorbate 20. In some embodiments, the non-ionic surfactant is Poloxamer 407. In some embodiments, the non-ionic surfactant is Tyloxapol. In some embodiments, the non-ionic surfactant is cremophor. The non-ionic surfactants described herein can be present within any one of the ranges (e.g., percent w/w) described herein, a specific value that falls within the described ranges.

In some embodiments, the composition further comprises cosolvents (e.g., between 0.5 and 50% w/w), such as N,N-Dimethylacetamide, ethanol, PEG-400, propylene glycol, dimethylsulfoxide (DMSO); oils, or cyclodextrins may be added to a pharmaceutical preparation. In some embodiments, the composition further comprises a tonicity-adjusting agent. In some embodiments, the tonicity-adjusting agent is mannitol, sorbitol, glucose or trehalose, or an inorganic salt such as sodium chloride. In some embodiments, the composition comprises mannitol. In some embodiments, the composition comprises sorbitol. In some embodiments, the composition comprises glucose or trehalose. In some embodiments, the composition comprises an inorganic salt. In some embodiments, the tonicity-adjusting agent is present at an amount suitable to bring the tonicity of the composition into the 250-400 mOsm/L range. In some embodiments, the non-ionic surfactant is about 1% w/w of the composition to about 10% w/w of the composition. In some embodiments, the non-ionic surfactant is at least about 1% w/w of the composition. In some embodiments, the non-ionic surfactant is at most about 10% w/w of the composition. In some embodiments, the non-ionic surfactant is about 1% w/w of the composition to about 2% w/w of the composition, about 1% w/w of the composition to about 3% w/w of the composition, about 1% w/w of the composition to about 4% w/w of the composition, about 1% w/w of the composition to about 5% w/w of the composition, about 1% w/w of the composition to about 10% w/w of the composition, about 2% w/w of the composition to about 3% w/w of the composition, about 2% w/w of the composition to about 4% w/w of the composition, about 2% w/w of the composition to about 5% w/w of the composition, about 2% w/w of the composition to about 10% w/w of the composition, about 3% w/w of the composition to about 4% w/w of the composition, about 3% w/w of the composition to about 5% w/w of the composition, about 3% w/w of the composition to about 10% w/w of the composition, about 4% w/w of the composition to about 5% w/w of the composition, about 4% w/w of the composition to about 10% w/w of the composition, or about 5% w/w of the composition to about 10% w/w of the composition. In some embodiments, the non-ionic surfactant is about 1% w/w of the composition, about 2% w/w of the composition, about 3% w/w of the composition, about 4% w/w of the composition, about 5% w/w of the composition, or about 10% w/w of the composition.

In some embodiments, the composition comprises a buffering agent. In some embodiments, the buffering agent is an acidifying agent. In some embodiments, the acidifying agent is an acetate buffer at pH 4.5. In some embodiments, the concentration acetate buffer pH 4.5 is about 10 micromolar (mM). Generally, the pH may be controlled by an appropriate buffer suitable for injection into the eye, for example the pH of the composition can be in the 3.0-7.5 range or 3.5-4.5 range.

As described herein, the composition can comprise one or more excipients. Accordingly, in some embodiments, the composition comprises a non-ionic surfactant, a tonicity-adjusting agent, and a buffering agent, in combination with the peptide. Any of the described excipients can be combined within the amounts and/or ranges described.

### Dosages and Dosing Regimens

In an embodiment, the compositions described herein comprise an amount of the peptide suitable to inhibit Fas-mediated inflammation and/or treat, inhibit, reduce, and/or prevent retinal degeneration or the symptoms thereof in an eye. As used herein, a dose or dosage includes and/or refers to the amount of therapeutic agent, such as the peptides described, in a composition (e.g., a composition for administering to an eye). A dose can refer to either (i) the peptide (parent compound) or the pharmaceutically-acceptable salt thereof. In some embodiments, the amount of the peptide in the composition (i.e., pharmaceutical composition) that is suitable for the methods described herein (e.g., treating retinal degeneration) ranges from 5 micrograms (ug) to 10,000 ug. The dosing forms comprising the compositions described herein are generally administered to the vitreous humor of an eye and can be further formulated for injection into the eye (e.g., intravitreal injection). In some embodiments, the amount of the peptide (e.g., a peptide comprising the amino acid sequence HHIYLGAVNYIY) or a pharmaceutically-acceptable salt thereof that is suitable for the methods described herein ranges from 5 ug to 10,000 ug. In some embodiments, the amount of the peptide comprising the amino acid sequence HHIYLGAVNYIY or variant sequence thereof, or a pharmaceutically-acceptable salt of the peptide that is suitable for the methods described herein ranges from 5 ug to 10,000 ug. In some embodiments, the amount of the peptide having the structure of Formula I or a pharmaceutically-acceptable salt thereof that is suitable for the methods described herein ranges from 5 ug to 10,000 ug. In some embodiments, the amount of the peptide having the structure of Formula III or a pharmaceutically-acceptable salt thereof that is suitable for the methods described herein ranges from 5 ug to 10,000 ug.

In some embodiments, the composition comprises about 5 ug of the peptide to about 10,000 ug of the peptide. In some embodiments, the composition comprises at least about 5 ug of the peptide. In some embodiments, the composition comprises at most about 10,000 ug of the peptide. In some embodiments, the composition comprises about 5 ug of the peptide to about 25 ug of the peptide, about 5 ug of the peptide to about 50 ug of the peptide, about 5 ug of the peptide to about 100 ug of the peptide, about 5 ug of the peptide to about 200 ug of the peptide, about 5 ug of the peptide to about 500 ug of the peptide, about 5 ug of the peptide to about 1,000 ug of the peptide, about 5 ug of the peptide to about 2,500 ug of the peptide, about 5 ug of the peptide to about 5,000 ug of the peptide, about 5 ug of the peptide to about 10,000 ug of the peptide, about 25 ug of the peptide to about 50 ug of the peptide, about 25 ug of the peptide to about 100 ug of the peptide, about 25 ug of the peptide to about 200 ug of the peptide, about 25 ug of the peptide to about 500 ug of the peptide, about 25 ug of the peptide to about 1,000 ug of the peptide, about 25 ug of the peptide to about 2,500 ug of the peptide, about 25 ug of the peptide to about 5,000 ug of the peptide, about 25 ug of the peptide to about 10,000 ug of the peptide, about 50 ug of the peptide to about 100 ug of the peptide, about 50 ug of the peptide to about 200 ug of the peptide, about 50 ug of the peptide to about 500 ug of the peptide, about 50 ug of the peptide to about 1,000 ug of the peptide, about 50 ug of the peptide to about 2,500 ug of the peptide, about 50 ug of the peptide to about 5,000 ug of the peptide, about 50 ug of the peptide to about 10,000 ug of the peptide, about 100 ug of the peptide to about 200 ug of the peptide, about 100 ug of the peptide to about 500 ug of the peptide, about 100 ug of the peptide to about 1,000 ug of the peptide, about 100 ug of the peptide to about 2,500 ug of the peptide, about 100 ug of the peptide to about 5,000 ug of the peptide, about 100 ug of the peptide to about 10,000 ug of the peptide, about 200 ug of the peptide to about 500 ug of the peptide, about 200 ug of the peptide to about 1,000 ug of the peptide, about 200 ug of the peptide to about 2,500 ug of the peptide, about 200 ug of the peptide to about 5,000 ug of the peptide, about 200 ug of the peptide to about 10,000 ug of the peptide, about 500 ug of the peptide to about 1,000 ug of the peptide, about 500 ug of the peptide to about 2,500 ug of the peptide, about 500 ug of the peptide to about 5,000 ug of the peptide, about 500 ug of the peptide to about 10,000 ug of the peptide, about 1,000 ug of the peptide to about 2,500 ug of the peptide, about 1,000 ug of the peptide to about 5,000 ug of the peptide, about 1,000 ug of the peptide to about 10,000 ug of the peptide, about 2,500 ug of the peptide to about 5,000 ug of the peptide, about 2,500 ug of the peptide to about 10,000 ug of the peptide, or about 5,000 ug of the peptide to about 10,000 ug of the peptide. In some embodiments, the composition comprises about 5 ug of the peptide, about 25 ug of the peptide, about 50 ug of the peptide, about 100 ug of the peptide, about 200 ug of the peptide, about 500 ug of the peptide, about 1,000 ug of the peptide, about 2,500 ug of the peptide, about 5,000 ug of the peptide, or about 10,000 ug of the peptide.

In some embodiments, a dose comprises about 5 ug of a pharmaceutically-acceptable salt of the peptide to about 10,000 ug of a pharmaceutically-acceptable salt of the peptide. In some embodiments, a dose comprises at least about 5 ug of a pharmaceutically-acceptable salt of the peptide. In some embodiments, a dose comprises at most about 10,000 ug of a pharmaceutically-acceptable salt of the peptide. In some embodiments, a dose comprises about 5 ug of a pharmaceutically-acceptable salt of the peptide to about 100 ug of a pharmaceutically-acceptable salt of the peptide, about 5 ug of a pharmaceutically-acceptable salt of the peptide to about 200 ug of a pharmaceutically-acceptable salt of the peptide, about 5 ug of a pharmaceutically-acceptable salt of the peptide to about 500 ug of a pharmaceutically-acceptable salt of the peptide, about 5 ug of a pharmaceutically-acceptable salt of the peptide to about 1,000 ug of a pharmaceutically-acceptable salt of the peptide, about 5 ug of a pharmaceutically-acceptable salt of the peptide to about 2,500 ug of a pharmaceutically-acceptable salt of the peptide, about 5 ug of a pharmaceutically-acceptable salt of the peptide to about 5,000 ug of a pharmaceutically-acceptable salt of the peptide, about 5 ug of a pharmaceutically-acceptable salt of the peptide to about 10,000 ug of a pharmaceutically-acceptable salt of the peptide, about 100 ug of a pharmaceutically-acceptable salt of the peptide to about 200 ug of a pharmaceutically-acceptable salt of the peptide, about 100 ug of a pharmaceutically-acceptable salt of the peptide to about 500 ug of a pharmaceutically-acceptable salt of the peptide, about 100 ug of a pharmaceutically-acceptable salt of the peptide to about 1,000 ug of a pharmaceutically-acceptable salt of the peptide, about 100 ug of a pharmaceutically-acceptable salt of the peptide to about 2,500 ug of a pharmaceutically-acceptable salt of the peptide, about 100 ug of a pharmaceutically-acceptable salt of the peptide to about 5,000 ug of a pharmaceutically-acceptable salt of the peptide, about 100 ug of a pharmaceutically-acceptable salt of the peptide to about 10,000 ug of a pharmaceutically-acceptable salt of the peptide, about 200 ug of a pharmaceutically-acceptable salt of the peptide to about 500 ug of a pharmaceutically-acceptable salt of the peptide, about 200 ug of a pharmaceutically-acceptable salt of the peptide to about 1,000 ug of a pharmaceutically-acceptable salt of the peptide, about 200 ug of a pharmaceutically-acceptable salt of the peptide to about 2,500 ug of a pharmaceutically-acceptable salt of the peptide, about 200 ug of a pharmaceutically-acceptable salt of the peptide to about 5,000 ug of a pharmaceutically-acceptable salt of the peptide, about 200 ug of a pharmaceutically-acceptable salt of the peptide to about 10,000 ug of a pharmaceutically-acceptable salt of the peptide, about 500 ug of a pharmaceutically-acceptable salt of the peptide to about 1,000 ug of a pharmaceutically-acceptable salt of the peptide, about 500 ug of a pharmaceutically-acceptable salt of the peptide to about 2,500 ug of a pharmaceutically-acceptable salt of the peptide, about 500 ug of a pharmaceutically-acceptable salt of the peptide to about 5,000 ug of a pharmaceutically-acceptable salt of the peptide, about 500 ug of a pharmaceutically-acceptable salt of the peptide to about 10,000 ug of a pharmaceutically-acceptable salt of the peptide, about 1,000 ug of a pharmaceutically-acceptable salt of the peptide to about 2,500 ug of a pharmaceutically-acceptable salt of the peptide, about 1,000 ug of a pharmaceutically-acceptable salt of the peptide to about 5,000 ug of a pharmaceutically-acceptable salt of the peptide, about 1,000 ug of a pharmaceutically-acceptable salt of the peptide to about 10,000 ug of a pharmaceutically-acceptable salt of the peptide, about 2,500 ug of a pharmaceutically-acceptable salt of the peptide to about 5,000 ug of a pharmaceutically-acceptable salt of the peptide, about 2,500 ug of a pharmaceutically-acceptable salt of the peptide to about 10,000 ug of a pharmaceutically-acceptable salt of the peptide, or about 5,000 ug of a pharmaceutically-acceptable salt of the peptide to about 10,000 ug of a pharmaceutically-acceptable salt of the peptide. In some embodiments, a dose comprises about 5 ug of a pharmaceutically-acceptable salt of the peptide, about 100 ug of a pharmaceutically-acceptable salt of the peptide, about 200 ug of a pharmaceutically-acceptable salt of the peptide, about 500 ug of a pharmaceutically-acceptable salt of the peptide, about 1,000 ug of a pharmaceutically-acceptable salt of the peptide, about 2,500 ug of a pharmaceutically-acceptable salt of the peptide, about 5,000 ug of a pharmaceutically-acceptable salt of the peptide, or about 10,000 ug of a pharmaceutically-acceptable salt of the peptide.

In some embodiments, the amount of the peptide comprising the amino acid sequence HHIYLGAVNYIY or variant sequence thereof, or a pharmaceutically-acceptable salt of the peptide that is suitable for the methods described herein ranges from 5 ug to 300 ug. In some embodiments, the amount of the peptide having the structure of Formula I or a pharmaceutically-acceptable salt thereof that is suitable for the methods described herein ranges from 5 ug to 300 ug. In some embodiments, the amount of the peptide having the structure of Formula III or a pharmaceutically-acceptable salt thereof that is suitable for the methods described herein ranges from 5 ug to 300 ug.

In some embodiments, the composition comprises about 5 ug of the peptide to about 300 ug of the peptide. In some embodiments, the composition comprises at least about 5 ug of the peptide. In some embodiments, the composition comprises at most about 300 ug of the peptide. In some embodiments, the composition comprises about 5 ug of the peptide to about 10 ug of the peptide, about 5 ug of the peptide to about 25 ug of the peptide, about 5 ug of the peptide to about 50 ug of the peptide, about 5 ug of the peptide to about 75 ug of the peptide, about 5 ug of the peptide to about 100 ug of the peptide, about 5 ug of the peptide to about 150 ug of the peptide, about 5 ug of the peptide to about 200 ug of the peptide, about 5 ug of the peptide to about 250 ug of the peptide, about 5 ug of the peptide to about 300 ug of the peptide, about 10 ug of the peptide to about 25 ug of the peptide, about 10 ug of the peptide to about 50 ug of the peptide, about 10 ug of the peptide to about 75 ug of the peptide, about 10 ug of the peptide to about 100 ug of the peptide, about 10 ug of the peptide to about 150 ug of the peptide, about 10 ug of the peptide to about 200 ug of the peptide, about 10 ug of the peptide to about 250 ug of the peptide, about 10 ug of the peptide to about 300 ug of the peptide, about 25 ug of the peptide to about 50 ug of the peptide, about 25 ug of the peptide to about 75 ug of the peptide, about 25 ug of the peptide to about 100 ug of the peptide, about 25 ug of the peptide to about 150 ug of the peptide, about 25 ug of the peptide to about 200 ug of the peptide, about 25 ug of the peptide to about 250 ug of the peptide, about 25 ug of the peptide to about 300 ug of the peptide, about 50 ug of the peptide to about 75 ug of the peptide, about 50 ug of the peptide to about 100 ug of the peptide, about 50 ug of the peptide to about 150 ug of the peptide, about 50 ug of the peptide to about 200 ug of the peptide, about 50 ug of the peptide to about 250 ug of the peptide, about 50 ug of the peptide to about 300 ug of the peptide, about 75 ug of the peptide to about 100 ug of the peptide, about 75 ug of the peptide to about 150 ug of the peptide, about 75 ug of the peptide to about 200 ug of the peptide, about 75 ug of the peptide to about 250 ug of the peptide, about 75 ug of the peptide to about 300 ug of the peptide, about 100 ug of the peptide to about 150 ug of the peptide, about 100 ug of the peptide to about 200 ug of the peptide, about 100 ug of the peptide to about 250 ug of the peptide, about 100 ug of the peptide to about 300 ug of the peptide, about 150 ug of the peptide to about 200 ug of the peptide, about 150 ug of the peptide to about 250 ug of the peptide, about 150 ug of the peptide to about 300 ug of the peptide, about 200 ug of the peptide to about 250 ug of the peptide, about 200 ug of the peptide to about 300 ug of the peptide, or about 250 ug of the peptide to about 300 ug of the peptide. In some embodiments, the composition comprises about 5 ug of the peptide, about 10 ug of the peptide, about 25 ug of the peptide, about 50 ug of the peptide, about 75 ug of the peptide, about 100 ug of the peptide, about 150 ug of the peptide, about 200 ug of the peptide, about 250 ug of the peptide, or about 300 ug of the peptide.

In some embodiments, a dose comprises about 5 ug of a pharmaceutically-acceptable salt of the peptide to about 300 ug of a pharmaceutically-acceptable salt of the peptide. In some embodiments, a dose comprises at least about 5 ug of a pharmaceutically-acceptable salt of the peptide. In some embodiments, a dose comprises at most about 300 ug of a pharmaceutically-acceptable salt of the peptide. In some embodiments, a dose comprises about 5 ug of a pharmaceutically-acceptable salt of the peptide to about 25 ug of a pharmaceutically-acceptable salt of the peptide, about 5 ug of a pharmaceutically-acceptable salt of the peptide to about 50 ug of a pharmaceutically-acceptable salt of the peptide, about 5 ug of a pharmaceutically-acceptable salt of the peptide to about 100 ug of a pharmaceutically-acceptable salt of the peptide, about 5 ug of a pharmaceutically-acceptable salt of the peptide to about 150 ug of a pharmaceutically-acceptable salt of the peptide, about 5 ug of a pharmaceutically-acceptable salt of the peptide to about 200 ug of a pharmaceutically-acceptable salt of the peptide, about 5 ug of a pharmaceutically-acceptable salt of the peptide to about 250 ug of a pharmaceutically-acceptable salt of the peptide, about 5 ug of a pharmaceutically-acceptable salt of the peptide to about 300 ug of a pharmaceutically-acceptable salt of the peptide, about 25 ug of a pharmaceutically-acceptable salt of the peptide to about 50 ug of a pharmaceutically-acceptable salt of the peptide, about 25 ug of a pharmaceutically-acceptable salt of the peptide to about 100 ug of a pharmaceutically-acceptable salt of the peptide, about 25 ug of a pharmaceutically-acceptable salt of the peptide to about 150 ug of a pharmaceutically-acceptable salt of the peptide, about 25 ug of a pharmaceutically-acceptable salt of the peptide to about 200 ug of a pharmaceutically-acceptable salt of the peptide, about 25 ug of a pharmaceutically-acceptable salt of the peptide to about 250 ug of a pharmaceutically-acceptable salt of the peptide, about 25 ug of a pharmaceutically-acceptable salt of the peptide to about 300 ug of a pharmaceutically-acceptable salt of the peptide, about 50 ug of a pharmaceutically-acceptable salt of the peptide to about 100 ug of a pharmaceutically-acceptable salt of the peptide, about 50 ug of a pharmaceutically-acceptable salt of the peptide to about 150 ug of a pharmaceutically-acceptable salt of the peptide, about 50 ug of a pharmaceutically-acceptable salt of the peptide to about 200 ug of a pharmaceutically-acceptable salt of the peptide, about 50 ug of a pharmaceutically-acceptable salt of the peptide to about 250 ug of a pharmaceutically-acceptable salt of the peptide, about 50 ug of a pharmaceutically-acceptable salt of the peptide to about 300 ug of a pharmaceutically-acceptable salt of the peptide, about 100 ug of a pharmaceutically-acceptable salt of the peptide to about 150 ug of a pharmaceutically-acceptable salt of the peptide, about 100 ug of a pharmaceutically-acceptable salt of the peptide to about 200 ug of a pharmaceutically-acceptable salt of the peptide, about 100 ug of a pharmaceutically-acceptable salt of the peptide to about 250 ug of a pharmaceutically-acceptable salt of the peptide, about 100 ug of a pharmaceutically-acceptable salt of the peptide to about 300 ug of a pharmaceutically-acceptable salt of the peptide, about 150 ug of a pharmaceutically-acceptable salt of the peptide to about 200 ug of a pharmaceutically-acceptable salt of the peptide, about 150 ug of a pharmaceutically-acceptable salt of the peptide to about 250 ug of a pharmaceutically-acceptable salt of the peptide, about 150 ug of a pharmaceutically-acceptable salt of the peptide to about 300 ug of a pharmaceutically-acceptable salt of the peptide, about 200 ug of a pharmaceutically-acceptable salt of the peptide to about 250 ug of a pharmaceutically-acceptable salt of the peptide, about 200 ug of a pharmaceutically-acceptable salt of the peptide to about 300 ug of a pharmaceutically-acceptable salt of the peptide, or about 250 ug of a pharmaceutically-acceptable salt of the peptide to about 300 ug of a pharmaceutically-acceptable salt of the peptide. In some embodiments, a dose comprises about 5 ug of a pharmaceutically-acceptable salt of the peptide, about 25 ug of a pharmaceutically-acceptable salt of the peptide, about 50 ug of a pharmaceutically-acceptable salt of the peptide, about 100 ug of a pharmaceutically-acceptable salt of the peptide, about 150 ug of a pharmaceutically-acceptable salt of the peptide, about 200 ug of a pharmaceutically-acceptable salt of the peptide, about 250 ug of a pharmaceutically-acceptable salt of the peptide, or about 300 ug of a pharmaceutically-acceptable salt of the peptide.

The concentration of the peptide within the composition can be adjusted in a manner suitable for ocular administration. In some embodiments, the concentration of the peptide within the composition ranges from about 0.1 milligrams per milliliter (mg/mL) to about 5 mg/mL. In some embodiments, the concentration of the peptide within the composition ranges from about 0.1 milligrams per milliliter (mg/mL) to about 10 mg/mL. In some embodiments, the concentration of the peptide within the composition ranges from about 0.1 milligrams per milliliter (mg/mL) to about 100 mg/mL. In some embodiments, the concentration of the peptide (e.g., a peptide comprising the amino acid sequence HHIYLGAVNYIY) or a pharmaceutically-acceptable salt thereof ranges from about 0.1 mg/mL to about 5 mg/mL. In some embodiments, the concentration of the peptide comprising the amino acid sequence HHIYLGAVNYIY or variant sequence thereof, or a pharmaceutically-acceptable salt of the peptide ranges from about 0.1 milligrams per milliliter (mg/mL) to about 5 mg/mL. In some embodiments, the concentration of the peptide having the structure of Formula I or a pharmaceutically-acceptable salt thereof ranges from about 0.1 mg/mL to about 5 mg/mL. In some embodiments, the concentration of the peptide having the structure of Formula III or a pharmaceutically-acceptable salt thereof ranges from about 0.1 mg/mL to about 5 mg/mL.

In some embodiments, the concentration of the peptide within the composition is about 0.1 mg/mL to about 5 mg/mL. In some embodiments, the concentration of the peptide within the composition is at least about 0.1 mg/mL. In some embodiments, the concentration of the peptide within the composition is at most about 5 mg/mL. In some embodiments, the concentration of the peptide within the composition is about 0.1 mg/mL to about 0.5 mg/mL, about 0.1 mg/mL to about 1 mg/mL, about 0.1 mg/mL to about 1.5 mg/mL, about 0.1 mg/mL to about 2 mg/mL, about 0.1 mg/mL to about 2.5 mg/mL, about 0.1 mg/mL to about 3 mg/mL, about 0.1 mg/mL to about 4 mg/mL, about 0.1 mg/mL to about 5 mg/mL, about 0.5 mg/mL to about 1 mg/mL, about 0.5 mg/mL to about 1.5 mg/mL, about 0.5 mg/mL to about 2 mg/mL, about 0.5 mg/mL to about 2.5 mg/mL, about 0.5 mg/mL to about 3 mg/mL, about 0.5 mg/mL to about 4 mg/mL, about 0.5 mg/mL to about 5 mg/mL, about 1 mg/mL to about 1.5 mg/mL, about 1 mg/mL to about 2 mg/mL, about 1 mg/mL to about 2.5 mg/mL, about 1 mg/mL to about 3 mg/mL, about 1 mg/mL to about 4 mg/mL, about 1 mg/mL to about 5 mg/mL, about 1.5 mg/mL to about 2 mg/mL, about 1.5 mg/mL to about 2.5 mg/mL, about 1.5 mg/mL to about 3 mg/mL, about 1.5 mg/mL to about 4 mg/mL, about 1.5 mg/mL to about 5 mg/mL, about 2 mg/mL to about 2.5 mg/mL, about 2 mg/mL to about 3 mg/mL, about 2 mg/mL to about 4 mg/mL, about 2 mg/mL to about 5 mg/mL, about 2.5 mg/mL to about 3 mg/mL, about 2.5 mg/mL to about 4 mg/mL, about 2.5 mg/mL to about 5 mg/mL, about 3 mg/mL to about 4 mg/mL, about 3 mg/mL to about 5 mg/mL, or about 4 mg/mL to about 5 mg/mL. In some embodiments, the concentration of the peptide within the composition is about 0.1 mg/mL, about 0.5 mg/mL, about 1 mg/mL, about 1.5 mg/mL, about 2 mg/mL, about 2.5 mg/mL, about 3 mg/mL, about 4 mg/mL, or about 5 mg/mL.

### Pharmacokinetics

As contemplated herein, the compositions described herein are administered to an eye of an individual in need thereof. Administration to an eye (i.e., “ocular application” or “ocular administration”) includes subconjunctival, intravitreal, retrobulbar, intracameral administration subretinal, or suprachoroidal. In some embodiments, ocular administration comprises subconjunctival, intravitreal, retrobulbar, or intracameral administration. In some embodiments, ocular administration comprises intravitreal administration. In some embodiments, ocular administration comprises subconjunctival administration. In some embodiments, ocular administration comprises retrobulbar administration. In some embodiments, ocular administration comprises intracameral administration.

In some embodiments, the dosing forms comprising the compositions described herein are generally administered to the vitreous humor of an eye. In some embodiments, the half-life of the peptide (e.g., a peptide comprising the amino acid sequence HHIYLGAVNYIY) or a pharmaceutically-acceptable salt thereof in the vitreous humor is greater than about 30 days to greater than about 275 days. In some embodiments, the peptide comprising the amino acid sequence HHIYLGAVNYIY or variant sequence thereof, or a pharmaceutically-acceptable salt of the peptide has a half-life in the vitreous humor that is greater than about 30 days to greater than about 275 days. In some embodiments, the peptide having the structure of Formula I or a pharmaceutically-acceptable salt thereof has a half-life in the vitreous humor that is greater than is greater than about 30 days to greater than about 275 days. In some embodiments, the peptide having the structure of Formula III or a pharmaceutically-acceptable salt thereof has a half-life in the vitreous humor that is greater than is greater than about 30 days to greater than about 275 days. In some embodiments, the peptide comprising the amino acid sequence HHIYLGAVNYIY or variant sequence thereof, or a pharmaceutically-acceptable salt of the peptide has a half-life in the vitreous humor that is greater than about 14 days to greater than about 275 days. In some embodiments, the peptide having the structure of Formula I or a pharmaceutically-acceptable salt thereof has a half-life in the vitreous humor that is greater than is greater than about 14 days to greater than about 275 days. In some embodiments, the peptide having the structure of Formula III or a pharmaceutically-acceptable salt thereof has a half-life in the vitreous humor that is greater than is greater than about 14 days to greater than about 275 days.

In some embodiments, the half-life of the peptide is greater than about 14 days in the eye. In some embodiments, the half-life of the peptide is greater than about 30 days in the eye. In some embodiments, the half-life of the peptide is greater than about 60 days in the eye. In some embodiments, the half-life of the peptide is greater than about 90 days in the eye. In some embodiments, the half-life of the peptide is greater than about 120 days in the eye. In some embodiments, the half-life of the peptide is greater than about 150 days in the eye. In some embodiments, the half-life of the peptide is greater than about 180 days in the eye. In some embodiments, the half-life of the peptide is greater than about 210 days in the eye. In some embodiments, the half-life of the peptide is greater than about 240 days in the eye. In some embodiments, the half-life of the peptide is greater than about 270 days in the eye.

In some embodiments, the half-life of the peptide is greater than about 14 days in the vitreous humor. In some embodiments, the half-life of the peptide is greater than about 30 days in the vitreous humor. In some embodiments, the half-life of the peptide is greater than about 60 days in the vitreous humor. In some embodiments, the half-life of the peptide is greater than about 90 days in the vitreous humor. In some embodiments, the half-life of the peptide is greater than about 120 days in the vitreous humor. In some embodiments, the half-life of the peptide is greater than about 150 days in the vitreous humor. In some embodiments, the half-life of the peptide is greater than about 180 days in the vitreous humor. In some embodiments, the half-life of the peptide is greater than about 210 days in the vitreous humor. In some embodiments, the half-life of the peptide is greater than about 240 days in the vitreous humor. In some embodiments, the half-life of the peptide is greater than about 270 days in the vitreous humor.

Determining the amount of the peptide in the vitreous humor generally requires collecting all of the vitreous fluid or a substantial portion thereof from an eye, or sacrificing the eye in order to sample the vitreous humor. In some embodiments, collecting all of the vitreous fluid or a substantial portion thereof in a human eye, or sacrificing an eye is not feasible for maintaining the health of an eye in a human. Accordingly, in some embodiments, the half-life of the peptide in a human eye is determined by measuring and/or extrapolating from a half-life of the peptide in the eye of a mammal. In some embodiments, the mammal is a rabbit. In some embodiments, the mammal is a pig (e.g., minipig). In some embodiments, the mammal is a monkey. Various methods of detecting the presence of a drug are also suitable for detecting the peptide. For example, methods suitable for detecting the peptide include performing mass spectrometry (e.g., liquid chromatography-mass spectrometry (LC-MS) or high-performance LC-MS (HPLC-MS)) on a sample from the vitreous humor.

In an embodiment, the vitreous humor functions as a depot to provide a therapeutically effective amount of the peptide. Assays for detecting the peptide in retinal tissue require sacrificing the eye. Variability in methods for measuring an amount of the peptide present in the retinal tissue generally make detecting the peptide difficult and generally underestimate the actual amount of the peptide in the retinal tissue. In certain instances, the amount of peptide detected in retinal tissue thereby represents the minimal value of the peptide in the retinal tissue. Therefore, in some embodiments, detection of the peptide in the vitreous humor is a proxy and/or a suitable indicator for the therapeutically effective amount that is provided to the retinal tissue. In some embodiments, the peptide is provided to the retina from the vitreous for at least about (n) days after administration. In some embodiments, the peptide is provided to the retina from the vitreous for at least about (n) days after administration, wherein a therapeutic effect of the peptide is maintained for a period longer than (n) days. In some embodiments, the therapeutic effect of the peptide is maintained for a period 2, 3, 4, 5, or 10 times longer than (n) days. In some embodiments, the peptide is provided to the retina from the vitreous humor for at least about (n) days after administration, wherein the peptide is detectable in a sample from retinal tissue of a mammal at day (n) after administration. In some embodiments, the peptide is provided to the retina from the vitreous for at least about (n) days after administration, wherein the peptide is detectable in a sample from retinal tissue of a mammal at day (n). In some embodiments, determining (n) in a human eye is determined by measuring and/or extrapolating (n) from the (n) value of the peptide in the eye of a mammal. In some embodiments, the mammal is a rabbit. In some embodiments, the mammal is a pig (e.g., minipig). In some embodiments, the mammal is a human. In some embodiments, the peptide comprising the amino acid sequence HHIYLGAVNYIY or variant sequence thereof, or a pharmaceutically-acceptable salt of the peptide is provided to the retina from the vitreous for at least about (n) days after administration. In some embodiments, the peptide comprising the amino acid sequence HHIYLGAVNYIY or variant sequence thereof, or a pharmaceutically-acceptable salt of the peptide is provided to the retina from the vitreous for at least about (n) days after administration, wherein the peptide is detectable in a sample from retinal tissue of a mammal at day (n). In some embodiments, the mammal is a rabbit. In some embodiments, the mammal is a pig (e.g., minipig). In some embodiments, the peptide having the structure of Formula I or a pharmaceutically-acceptable salt thereof is provided to the retina from the vitreous for at least about (n) days after administration. In some embodiments, the peptide having the structure of Formula I or a pharmaceutically-acceptable salt thereof is provided to the retina from the vitreous for at least about (n) days after administration, wherein the peptide is detectable in a sample from retinal tissue of a mammal at day (n). In some embodiments, the mammal is a rabbit. In some embodiments, the mammal is a pig (e.g., minipig). In some embodiments, the peptide having the structure of Formula III or a pharmaceutically-acceptable salt thereof is provided to the retina from the vitreous for at least about (n) days after administration. In some embodiments, the peptide is provided to the retina from the vitreous for at least about (n) days after administration, wherein the peptide having the structure of Formula III or a pharmaceutically-acceptable salt thereof is detectable in a sample from retinal tissue of a mammal at day (n).

In some embodiments, (n) is about 30 to about 360. In some embodiments, (n) is at least about 30. In some embodiments, (n) is about 30 to about 60, about 30 to about 90, about 30 to about 120, about 30 to about 150, about 30 to about 180, about 30 to about 210, about 30 to about 240, about 30 to about 270, about 30 to about 300, about 30 to about 330, about 30 to about 360, about 60 to about 90, about 60 to about 120, about 60 to about 150, about 60 to about 180, about 60 to about 210, about 60 to about 240, about 60 to about 270, about 60 to about 300, about 60 to about 330, about 60 to about 360, about 90 to about 120, about 90 to about 150, about 90 to about 180, about 90 to about 210, about 90 to about 240, about 90 to about 270, about 90 to about 300, about 90 to about 330, about 90 to about 360, about 120 to about 150, about 120 to about 180, about 120 to about 210, about 120 to about 240, about 120 to about 270, about 120 to about 300, about 120 to about 330, about 120 to about 360, about 150 to about 180, about 150 to about 210, about 150 to about 240, about 150 to about 270, about 150 to about 300, about 150 to about 330, about 150 to about 360, about 180 to about 210, about 180 to about 240, about 180 to about 270, about 180 to about 300, about 180 to about 330, about 180 to about 360, about 210 to about 240, about 210 to about 270, about 210 to about 300, about 210 to about 330, about 210 to about 360, about 240 to about 270, about 240 to about 300, about 240 to about 330, about 240 to about 360, about 270 to about 300, about 270 to about 330, about 270 to about 360, about 300 to about 330, about 300 to about 360, or about 330 to about 360. In some embodiments, (n) is about 30, about 60, about 90, about 120, about 150, about 180, about 210, about 240, about 270, about 300, about 330, or about 360.

### Methods

Provided herein are methods that are advantageous in that the methods, generally, do not require frequently repeated injections into an eye (e.g., having an ocular disease, disorder, or condition, or a symptom thereof). In some embodiments, the methods described herein comprise administering a composition comprising the peptide to the vitreous humor of an eye. In some embodiments, the described herein comprise are uses of a composition comprising the peptide in a method of administering the composition to the vitreous humor of an eye. In some embodiments, the method is a method of treating inflammation (e.g., Fas-mediated inflammation) in an eye (e.g., the retinal tissue of an eye). In some embodiments, the method is a method of inhibiting, reducing, and/or preventing inflammation (e.g., Fas-mediated inflammation) in an eye (e.g., the retinal tissue of an eye). In some embodiments, the method includes treating symptoms associated with inflammation in the eye (e.g., increased intraocular pressure, loss of and/or decrease in visual acuity, blurred vision, distorted vision, etc.). In some embodiments, retinal inflammation can be determined by observing the symptoms associated with inflammation in the eye (e.g., retinal degeneration, increased intraocular pressure, loss of and/or decrease in visual acuity, blurred vision, distorted vision, etc.) and/or by a biological assay detecting the presence of inflammatory molecules (e.g., inflammatory cytokines) in a sample (e.g., vitreous humor sample) taken from the eye. Exemplary inflammatory molecules include, but are not limited to, Fas-mediated inflammation-related molecules (e.g. TNFa, IL-1b, IP-10, IL-18, MIPIa, IL-6, GFAP, MIP2, MCP-1, or MIP-1b); a Fas-mediated complement-related molecules (complement component 3 (C3) or complement component I q (C1q)) Caspase 8; components of the inflammasome (e.g., NLRP3 or NLRP2); C—X—C motif chemokines (e.g., CXCL2 (MIP-2 alpha) or CXCL10 (IP-10)); C-X3-C motif chemokines (e.g., CX3CL1 (fractalkine)); C—C motif chemokines (CCL2 (MCP-1), CCL3 (MIP-1a), and CCL4 (MIP-1b)); toll-like receptor 4 (TLR4); interleukin cytokines (e.g., IL-1b, IL-18, and IL-6); TNF superfamily cytokines (e.g., TNFa); or GFAP.

In some embodiments, the method is a method of treating retinal cell loss (e.g., retinal pigment epithelial cells, retinal ganglion cells, and/or photoreceptors) in an eye (e.g., the retinal tissue of an eye). In some embodiments, the method is a method of inhibiting, reducing, and/or preventing retinal cell loss (e.g., retinal pigment epithelial cells, retinal ganglion cells, and/or photoreceptors) in an eye (e.g., the retinal tissue of an eye). In some embodiments, the method includes treating symptoms associated with retinal cell loss (e.g., retinal pigment epithelial cells, retinal ganglion cells, and/or photoreceptors) in the eye (e.g., retinal degeneration, increased intraocular pressure, loss of and/or decrease in visual acuity, blurred vision, distorted vision, etc.).

In an embodiment, treating inflammation in the eye and/or retinal cell loss is useful for treating for retinal degeneration. In another embodiment, inhibiting, reducing, and/or preventing inflammation in the eye and/or retinal cell loss is useful for treating, inhibiting, reducing, and/or preventing retinal degeneration. As used herein, retinal degeneration includes and/or refers to the loss cells within the retina and/or death of cells within the retina. As used herein, retinal degeneration disease or disorder includes and/or refers to a disease associated with deterioration of the retina caused by the death and/or loss of the cells of the retinal tissue. In some embodiments, the method is a method of treating retinal degeneration. In some embodiments, the method is a method of inhibiting, reducing, and/or preventing retinal degeneration. In some embodiments, the method includes treating symptoms associated with retinal degeneration (e.g., increased intraocular pressure, loss of and/or decrease in visual acuity, blurred vision, distorted vision, etc.). In some embodiments, retinal degeneration comprises the loss and/or death of retinal pigment epithelial cells, retinal ganglion cells, photoreceptors, or a combination thereof. In some embodiments, retinal degeneration comprises anatomical degeneration In some embodiments, retinal degeneration comprises the loss and/or death of retinal pigment epithelial cells. In some embodiments, retinal degeneration comprises the loss and/or death of retinal ganglion cells. In some embodiments, retinal degeneration comprises the loss and/or death of photoreceptors. In some embodiments, retinal degeneration comprises the loss and/or death a combination of retinal pigment epithelial cells, retinal ganglion cells, and/or photoreceptors.

In an embodiment, treating (1) inflammation in the eye, (2) retinal cell loss, and/or (3) retinal degeneration is useful for treating an ocular disease, disorder, or condition. In another embodiment, inhibiting, reducing, and/or preventing (1) inflammation in the eye, (2) retinal cell loss, and/or (3) retinal degeneration is useful for treating, inhibiting, reducing the pathology of, and/or preventing an ocular disease, disorder, or condition. As used herein, ocular disease, disorder, or condition generally includes and/or refers to a disorder or pathological condition of the eye which is not normal to an eye in a healthy state. In some embodiments, the method is a method of treating an ocular disease, disorder, or condition. In some embodiments, the method is a method of inhibiting, reducing, and/or preventing an ocular disease, disorder, or condition. In some embodiments, the method includes treating symptoms associated with an ocular disease, disorder, or condition (e.g., retinal degeneration, increased intraocular pressure, loss of and/or decrease in visual acuity, blurred vision, distorted vision, etc.). In some embodiments, the ocular disease, disorder, or condition is a chronic ocular disease, disorder, or condition. In some embodiments, the ocular disease, disorder, or condition comprises the loss and/or death of retinal pigment epithelial cells, retinal ganglion cells, photoreceptors, or a combination thereof. In some embodiments, the ocular disease, disorder, or condition comprises the loss and/or death of retinal pigment epithelial cells. In some embodiments, the ocular disease, disorder, or condition comprises the loss and/or death of retinal ganglion cells. In some embodiments, the ocular disease, disorder, or condition comprises the loss and/or death of photoreceptors. In some embodiments, the ocular disease, disorder, or condition comprises the loss and/or death a combination of retinal pigment epithelial cells, retinal ganglion cells, and/or photoreceptors.

In an embodiment, treating (1) inflammation in the eye, (2) retinal cell loss, and/or (3) retinal degeneration is useful for treating glaucoma. In another embodiment, inhibiting, reducing, and/or preventing (1) inflammation in the eye, (2) retinal cell loss, and/or (3) retinal degeneration is useful for treating, inhibiting, reducing the pathology of, and/or preventing glaucoma. As used herein, glaucoma generally includes and refers to a group of ocular diseases, which cause progressive damage to the optic nerve and resultant optical field defects, vision loss and, in some cases, blindness. In some embodiments, glaucoma is accompanied by abnormally high intraocular pressure and/or inflammation in the eye (e.g., within the retinal tissue). In some embodiments, glaucoma is not accompanied by abnormally high intraocular pressure and/or inflammation in the eye (e.g., within the retinal tissue). In some embodiments, the method is a method of treating glaucoma. In some embodiments, the method is a method of inhibiting, reducing, and/or preventing glaucoma. In some embodiments, the method includes treating symptoms associated with glaucoma (e.g., retinal degeneration, increased intraocular pressure, loss of and/or decrease in visual acuity, blurred vision, distorted vision, etc.). In some embodiments, the ocular disease, disorder, or condition comprises the loss and/or death of retinal ganglion cells.

In an embodiment, treating (1) inflammation in the eye, (2) retinal cell loss, and/or (3) retinal degeneration is useful for treating macular degeneration. In another embodiment, inhibiting, reducing, and/or preventing (1) inflammation in the eye, (2) retinal cell loss, and/or (3) retinal degeneration is useful for treating, inhibiting, reducing the pathology of, and/or preventing macular degeneration. In some embodiments, the method is a method of treating macular degeneration. In some embodiments, the method is a method of inhibiting, reducing, and/or preventing macular degeneration. As used herein, macular degeneration generally includes and/or refers to any of a number of conditions in which the retinal macula degenerates and/or becomes dysfunctional (e.g., as a consequence of decreased growth of cells of the macula, increased death or rearrangement of the cells of the macula (e.g., RPE cells), loss of normal biological function, or a combination thereof). Macular degeneration generally, results in the loss of integrity of the histological architecture of the cells and/or extracellular matrix of the normal macula and/or the loss of function of the cells of the macula. Examples of macular degeneration-related disorder include AMD, North Carolina macular dystrophy, Sorsby's fundus dystrophy, Stargardt's disease, pattern dystrophy, Best disease, dominant drusen, and malattia leventinese (radial drusen). As used herein, macular degeneration also includes and/or refers to extramacular changes that occur prior to, or following dysfunction and/or degeneration of the macula. In some embodiments, the method is a method of treating macular degeneration. In some embodiments, the method is a method of inhibiting, reducing, and/or preventing macular degeneration. In some embodiments, the method includes treating symptoms associated with macular degeneration (e.g., retinal degeneration, increased intraocular pressure, loss of and/or decrease in visual acuity, blurred vision, distorted vision, etc.). In some embodiments, the macular degeneration is age-related macular degeneration, non-exudative age-related macular degeneration, or exudative age-related macular degeneration. In some embodiments, the macular degeneration is age-related macular degeneration. In some embodiments, the macular degeneration is non-exudative age-related macular degeneration. In some embodiments, the macular degeneration is exudative age-related macular degeneration. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors.

In an embodiment, treating (1) inflammation in the eye, (2) retinal cell loss, and/or (3) retinal degeneration is useful for treating retinal detachment. In another embodiment, inhibiting, reducing, and/or preventing (1) inflammation in the eye, (2) retinal cell loss, and/or (3) retinal degeneration is useful for treating, inhibiting, reducing the pathology of, and/or preventing retinal detachment. As used herein, retinal detachment generally includes and refers to a condition and/or disorder of the eye in which the retina detaches (e.g., peels away, separates, etc.) from the underlying layer of support tissue. In some embodiments, the method is a method of treating retinal detachment. In some embodiments, the method is a method of inhibiting, reducing, and/or preventing retinal detachment. In some embodiments, the method includes treating symptoms associated retinal detachment (e.g., retinal degeneration, increased intraocular pressure, loss of and/or decrease in visual acuity, blurred vision, distorted vision, etc.). In some embodiments, the retinal detachment is chronic retinal detachment. In some embodiments, retinal detachment comprises the loss and/or death of retinal pigment epithelial cells, retinal ganglion cells, photoreceptors, or a combination thereof. In some embodiments, retinal detachment comprises the loss and/or death of retinal pigment epithelial cells. In some embodiments, retinal detachment comprises the loss and/or death of retinal ganglion cells. In some embodiments, retinal detachment comprises the loss and/or death of photoreceptors. In some embodiments, retinal detachment comprises the loss and/or death a combination of retinal pigment epithelial cells, retinal ganglion cells, and/or photoreceptors.

In an embodiment, treating (1) inflammation in the eye, (2) retinal cell loss, and/or (3) retinal degeneration is useful for treating ocular hypertension (e.g., elevated intraocular pressure). In another embodiment, inhibiting, reducing, and/or preventing (1) inflammation in the eye, (2) retinal cell loss, and/or (3) retinal degeneration is useful for treating, inhibiting, reducing the pathology of, and/or preventing ocular hypertension (e.g., elevated intraocular pressure). In some embodiments, the method is a method of treating ocular hypertension (e.g., elevated intraocular pressure). In some embodiments, the method is a method of treating ocular hypertension (e.g., elevated intraocular pressure). In some embodiments, ocular hypertension is characterized by an intraocular pressures of 21 mm Hg or greater. In some embodiments, ocular hypertension includes by an intraocular pressures of 18 mm Hg or greater.

In an embodiment, treating (1) inflammation in the eye, (2) retinal cell loss, and/or (3) retinal degeneration is useful for treating ocular ischemia. In another embodiment, inhibiting, reducing, and/or preventing (1) inflammation in the eye, (2) retinal cell loss, and/or (3) retinal degeneration is useful for treating, inhibiting, reducing the pathology of, and/or preventing ocular ischemia. In some embodiments, the method is a method of treating ocular ischemia. In some embodiments, the method is a method of treating ocular hypertension or the effects thereof (e.g., retinal degeneration).

In an embodiment, treating (1) inflammation in the eye, (2) retinal cell loss, and/or (3) retinal degeneration is useful for treating a loss in visual acuity. In another embodiment, inhibiting, reducing, and/or preventing (1) inflammation in the eye, (2) retinal cell loss, and/or (3) retinal degeneration is useful for treating, inhibiting, reducing the pathology of, and/or preventing a loss in visual acuity. In some embodiments, the method is a method of treating a loss in visual acuity. In some embodiments, the method is a method of treating a loss in visual acuity. In some embodiments, a loss in visual acuity is characterized by and/or equivalent to a reduction in visual acuity (e.g., a reduction from 20/100 to 20/200, or a reduction from 20/40 to 20/50, etc.).

In an embodiment, treating (1) inflammation in the eye, (2) retinal cell loss, and/or (3) retinal degeneration is useful for treating a loss in visual function (e.g., a loss in visual acuity, field of vision, contrast sensitivity, binocular function, and/or light/dark adaptation). In another embodiment, inhibiting, reducing, and/or preventing (1) inflammation in the eye, (2) retinal cell loss, and/or (3) retinal degeneration is useful for treating, inhibiting, reducing the pathology of, and/or preventing a loss in visual function. In some embodiments, the method is a method of treating a loss in visual function. In some embodiments, the method is a method of treating a loss in visual function. In some embodiments, a loss in visual function is characterized by and/or equivalent to a reduction in visual function assessments (e.g., visual acuity, field of vision, contrast sensitivity, binocular function, and/or light/dark adaptation).

In some embodiments, the method comprises administering a composition comprising the peptide (e.g., a peptide comprising the amino acid sequence HHIYLGAVNYIY) or a pharmaceutically-acceptable salt thereof. In some embodiments, the method comprises administering a composition comprising the peptide comprising the amino acid sequence HHIYLGAVNYIY or variant sequence thereof, or a pharmaceutically-acceptable salt of the peptide. In some embodiments, the method comprises administering a composition comprising the peptide having the structure of any one of Formulas I-IX or a pharmaceutically-acceptable salt thereof. In some embodiments, the method comprises administering a composition comprising the peptide having the structure of Formula I or a pharmaceutically-acceptable salt thereof. In some embodiments, the method comprises administering a composition comprising the peptide having the structure of Formula III or a pharmaceutically-acceptable salt thereof. Provided is the use of a composition comprising a peptide (e.g., a peptide comprising the amino acid sequence HHIYLGAVNYIY) or a pharmaceutically-acceptable salt thereof in the methods described herein. Also provided is the use of a composition comprising a peptide comprising the amino acid sequence HHIYLGAVNYIY or variant sequence thereof, or a pharmaceutically-acceptable salt of the peptide in the methods described herein. Further provided is the use of a composition comprising a peptide having the structure of Formula I or a pharmaceutically-acceptable salt thereof in the methods described herein. Further provided is the use of a composition comprising a peptide having the structure of Formula III or a pharmaceutically-acceptable salt thereof in the methods described herein.

Provided are also methods, comprising: administering a composition comprising a peptide comprising an amino acid sequence HHIYLGAVNYIY or a variant sequence thereof, or a pharmaceutically-acceptable salt of the peptide, to a vitreous humor of an eye thereby providing the peptide to retinal tissue in the eye, whereby the peptide has a half-life in the vitreous humor of at least about (z) days. Also provided are also methods, comprising: administering a composition comprising a peptide having the structure of Formula I or a pharmaceutically-acceptable thereof to a vitreous humor of an eye thereby providing the peptide to retinal tissue in the eye, whereby the peptide has a half-life in the vitreous humor of at least about (z) days. Further provided are also methods, comprising: administering a composition comprising a peptide having the structure of Formula III or a pharmaceutically-acceptable thereof to a vitreous humor of an eye thereby providing the peptide to retinal tissue in the eye, whereby the peptide has a half-life in the vitreous humor of at least about (z) days.

Provided is the use of a composition comprising a peptide comprising the amino acid sequence HHIYLGAVNYIY or variant sequence thereof, or a pharmaceutically-acceptable salt of the peptide in a method comprising: administering the composition to a vitreous humor of an eye thereby providing the peptide to retinal tissue in the eye, whereby the peptide has a half-life in the vitreous humor of at least about (z) days. Also provided is the use of a composition comprising a peptide having the structure of Formula I or a pharmaceutically-acceptable salt thereof in a method comprising: administering the composition to a vitreous humor of an eye thereby providing the peptide to retinal tissue in the eye, whereby the peptide has a half-life in the vitreous humor of at least about (z) days. Further provided is the use of a composition comprising a peptide having the structure of Formula III or a pharmaceutically-acceptable salt thereof in a method comprising: administering the composition to a vitreous humor of an eye thereby providing the peptide to retinal tissue in the eye, whereby the peptide has a half-life in the vitreous humor of at least about (z) days.

Provided is a composition comprising a peptide comprising the amino acid sequence HHIYLGAVNYIY or variant sequence thereof, or a pharmaceutically-acceptable salt of the peptide, for use in a method comprising: administering the composition to a vitreous humor of an eye thereby providing the peptide to retinal tissue in the eye, whereby the peptide has a half-life in the vitreous humor of at least about (z) days. Also provided is a composition comprising a peptide having the structure of Formula I or a pharmaceutically-acceptable salt thereof for use in a method comprising: administering the composition to a vitreous humor of an eye thereby providing the peptide to retinal tissue in the eye, whereby the peptide has a half-life in the vitreous humor of at least about (z) days. Further provided is a composition comprising a peptide having the structure of Formula III or a pharmaceutically-acceptable salt thereof for use in a method comprising: administering the composition to a vitreous humor of an eye thereby providing the peptide to retinal tissue in the eye, whereby the peptide has a half-life in the vitreous humor of at least about (z) days.

In some embodiments, the method is a method of treating inflammation (e.g., Fas-mediated inflammation) in an eye (e.g., the retinal tissue of an eye). In some embodiments, the method is a method of inhibiting, reducing, and/or preventing inflammation (e.g., Fas-mediated inflammation) in an eye (e.g., the retinal tissue of an eye). In some embodiments, the method includes treating symptoms associated with inflammation in the eye (e.g., increased intraocular pressure, loss of and/or decrease in visual acuity, blurred vision, distorted vision, etc.). In some embodiments, retinal inflammation can be determined by observing the symptoms associated with inflammation in the eye (e.g., retinal degeneration, increased intraocular pressure, loss of and/or decrease in visual acuity, blurred vision, distorted vision, etc.) and/or by a biological assay detecting the presence of inflammatory molecules (e.g., inflammatory cytokines) in a sample (e.g., vitreous humor sample) taken from the eye.

In some embodiments, the method is a method of treating retinal cell loss (e.g., retinal pigment epithelial cells, retinal ganglion cells, and/or photoreceptors) in an eye (e.g., the retinal tissue of an eye). In some embodiments, the method is a method of inhibiting, reducing, and/or preventing retinal cell loss (e.g., retinal pigment epithelial cells, retinal ganglion cells, and/or photoreceptors) in an eye (e.g., the retinal tissue of an eye). In some embodiments, the method includes treating symptoms associated with retinal cell loss (e.g., retinal pigment epithelial cells, retinal ganglion cells, and/or photoreceptors) in the eye (e.g., retinal degeneration, increased intraocular pressure, loss of and/or decrease in visual acuity, blurred vision, distorted vision, etc.).

In some embodiments, the method is a method of treating retinal degeneration. In some embodiments, the method is a method of inhibiting, reducing, and/or preventing retinal degeneration. In some embodiments, the method includes treating symptoms associated with retinal degeneration (e.g., increased intraocular pressure, loss of and/or decrease in visual acuity, blurred vision, distorted vision, etc.). In some embodiments, retinal degeneration comprises the loss and/or death of retinal pigment epithelial cells, retinal ganglion cells, photoreceptors, or a combination thereof. In some embodiments, retinal degeneration comprises the loss and/or death of retinal pigment epithelial cells. In some embodiments, retinal degeneration comprises the loss and/or death of retinal ganglion cells. In some embodiments, retinal degeneration comprises the loss and/or death of photoreceptors. In some embodiments, retinal degeneration comprises the loss and/or death a combination of retinal pigment epithelial cells, retinal ganglion cells, and/or photoreceptors.

In some embodiments, the method is a method of treating an ocular disease, disorder, or condition. In some embodiments, the method is a method of inhibiting, reducing, and/or preventing an ocular disease, disorder, or condition. In some embodiments, the method includes treating symptoms associated with an ocular disease, disorder, or condition (e.g., retinal degeneration, increased intraocular pressure, loss of and/or decrease in visual acuity, blurred vision, distorted vision, etc.). In some embodiments, the ocular disease, disorder, or condition is a chronic ocular disease, disorder, or condition. In some embodiments, the ocular disease, disorder, or condition comprises the loss and/or death of retinal pigment epithelial cells, retinal ganglion cells, photoreceptors, or a combination thereof. In some embodiments, the ocular disease, disorder, or condition comprises the loss and/or death of retinal pigment epithelial cells. In some embodiments, the ocular disease, disorder, or condition comprises the loss and/or death of retinal ganglion cells. In some embodiments, the ocular disease, disorder, or condition comprises the loss and/or death of photoreceptors. In some embodiments, the ocular disease, disorder, or condition comprises the loss and/or death a combination of retinal pigment epithelial cells, retinal ganglion cells, and/or photoreceptors.

In some embodiments, the method is a method of treating glaucoma. In some embodiments, the method is a method of inhibiting, reducing, and/or preventing glaucoma. In some embodiments, the method includes treating symptoms associated with glaucoma (e.g., retinal degeneration, increased intraocular pressure, loss of and/or decrease in visual acuity, blurred vision, distorted vision, etc.). In some embodiments, the ocular disease, disorder, or condition comprises the loss and/or death of retinal ganglion cells.

In some embodiments, the method is a method of treating macular degeneration. In some embodiments, the method is a method of inhibiting, reducing, and/or preventing macular degeneration. In some embodiments, the method includes treating symptoms associated with macular degeneration (e.g., retinal degeneration, increased intraocular pressure, loss of and/or decrease in visual acuity, blurred vision, distorted vision, etc.). In some embodiments, the macular degeneration is age-related macular degeneration, non-exudative age-related macular degeneration, or exudative age-related macular degeneration. In some embodiments, the macular degeneration is age-related macular degeneration. In some embodiments, the macular degeneration is non-exudative age-related macular degeneration. In some embodiments, the macular degeneration is exudative age-related macular degeneration. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors.

In some embodiments, the method is a method of treating retinal detachment. In some embodiments, the method is a method of inhibiting, reducing, and/or preventing retinal detachment. In some embodiments, the method includes treating symptoms associated retinal detachment (e.g., retinal degeneration, increased intraocular pressure, loss of and/or decrease in visual acuity, blurred vision, distorted vision, etc.). In some embodiments, the retinal detachment is chronic retinal detachment. In some embodiments, retinal detachment comprises the loss and/or death of retinal pigment epithelial cells, retinal ganglion cells, photoreceptors, or a combination thereof. In some embodiments, retinal detachment comprises the loss and/or death of retinal pigment epithelial cells. In some embodiments, retinal detachment comprises the loss and/or death of retinal ganglion cells. In some embodiments, retinal detachment comprises the loss and/or death of photoreceptors. In some embodiments, retinal detachment comprises the loss and/or death a combination of retinal pigment epithelial cells, retinal ganglion cells, and/or photoreceptors.

In some embodiments, the method is a method of treating a loss in visual acuity. In some embodiments, the method is a method of treating a loss in visual acuity. In some embodiments, a loss in visual acuity is characterized by and/or equivalent to a reduction in visual acuity (e.g., a reduction from 20/100 to 20/200, or a reduction from 20/40 to 20/50, etc.). In some embodiments, the method is a method of treating ocular hypertension (e.g., elevated intraocular pressure). In some embodiments, the method is a method of treating ocular hypertension (e.g., elevated intraocular pressure). In some embodiments, ocular hypertension is characterized by an intraocular pressures of 21 mm Hg or greater. In some embodiments, ocular hypertension includes by an intraocular pressures of 18 mm Hg or greater.

In some embodiments, the method is a method of treating a loss in visual function. In some embodiments, the method is a method of treating a loss in visual function. In some embodiments, a loss in visual function is characterized by and/or equivalent to a reduction in visual function assessments (e.g., visual acuity, field of vision, contrast sensitivity, binocular function, and/or light/dark adaptation).

Table 1 and Table 2 in Example 1 and Example 3 show pharmacokinetic data for a peptide comprising an amino acid sequence HHIYLGAVNYIY. The peptide is datable in the vitreous humor and retinal tissue at 168 days after intravitreal injection. The half-life in Table 1 is 279 days on the vitreous. The In some embodiments, (z) is 30 to 270. In some embodiments, it is not feasible to sample the vitreous humor of human eye. In such instances, the half-life can therefore be analogous to a pig eye or a rabbit eye. Accordingly, in some embodiments, the peptide has a half-life in the vitreous humor of a pig eye of at least about (z) days. In some embodiments, the peptide has a half-life in the vitreous humor of a rabbit eye of at least about (z) days. In some embodiments, (z) is 30 to 270. In some embodiments, (z) is at least 14. In some embodiments, (z) is at least 30. In some embodiments, (z) is at least 60. In some embodiments, (z) is at least 90. In some embodiments, (z) is at least 120. In some embodiments, (z) is at least 150. In some embodiments, (z) is at least 180. In some embodiments, (z) is at least 210. In some embodiments, (z) is at least 240. In some embodiments, (z) is at least 270. In some embodiments, (z) is greater than 30. In some embodiments, (z) is greater than 60. In some embodiments, (z) is greater than 90. In some embodiments, (z) is greater than 120. In some embodiments, (z) is greater than 150. In some embodiments, (z) is greater than 180. In some embodiments, (z) is greater than 210. In some embodiments, (z) is greater than 240. In some embodiments, (z) is greater than 270. In some embodiments, (z) is 30 to 60, 30 to 90, 30 to 120, 30 to 150, 30 to 180, 30 to 210, 30 to 240, 30 to 270, 60 to 90, 60 to 120, 60 to 150, 60 to 180, 60 to 210, 60 to 240, 60 to 270, 90 to 120, 90 to 150, 90 to 180, 90 to 210, 90 to 240, 90 to 270, 120 to 150, 120 to 180, 120 to 210, 120 to 240, 120 to 270, 150 to 180, 150 to 210, 150 to 240, 150 to 270, 180 to 210, 180 to 240, 180 to 270, 210 to 240, 210 to 270, or 240 to 270. In some embodiments, (z) is 14, 30, 60, 90, 120, 150, 180, 210, 240, or 270.

In some embodiments, the method comprises using the vitreous humor as a depot to provide a therapeutically-effective amount of the peptide to the retinal tissue. In some embodiments, the peptide is provided to the retina from the vitreous for at least about (n) days after administration. In some embodiments, the peptide is provided to the retina from the vitreous for at least about (n) days after administration, wherein the peptide is detectable in the retina at day (n). In some embodiments, the peptide is provided to the retina from the vitreous for at least about (n) days after administration, wherein the peptide is detectable in the retina at day (n)±10 days. In some embodiments, it is not feasible to sample the vitreous humor of an individual receiving the peptide. In such instances, a comparison can therefore be made to a (n) value generated from a pig eye or a rabbit eye. Accordingly, in some embodiments, wherein the peptide is detectable in the retina corresponds to detection in a pig eye or rabbit eye at in the retina at day (n)±10 days, or at day (n). In some embodiments, the peptide is detectable by mass-spectrometry from a sample taken from the retina.

In some embodiments, (n) is about 30 to about 365. In some embodiments, (n) is at least 14. In some embodiments, (n) is at least 30. In some embodiments, (n) is at least 60. In some embodiments, (n) is at least 90. In some embodiments, (n) is at least 120. In some embodiments, (n) is at least 150. In some embodiments, (n) is at least 180. In some embodiments, (n) is at least 210. In some embodiments, (n) is at least 240. In some embodiments, (n) is at least 270. In some embodiments, (n) is at least 300. In some embodiments, (n) is at least 330. In some embodiments, (n) is at least 365. In some embodiments, (n) is greater than 30. In some embodiments, (n) is greater than 60. In some embodiments, (n) is greater than 90. In some embodiments, (n) is greater than 120. In some embodiments, (n) is greater than 150. In some embodiments, (n) is greater than 180. In some embodiments, (n) is greater than 210. In some embodiments, (n) is greater than 240. In some embodiments, (n) is greater than 270. In some embodiments, (n) is greater than 300. In some embodiments, (n) is greater than 330. In some embodiments, (n) is greater than 365. In some embodiments, (n) is 30 to 60, 30 to 90, 30 to 120, 30 to 150, 30 to 180, 30 to 210, 30 to 240, 30 to 270, 30 to 300, 30 to 330, 30 to 365, 60 to 90, 60 to 120, 60 to 150, 60 to 180, 60 to 210, 60 to 240, 60 to 270, 60 to 300, 60 to 330, 60 to 365, 90 to 120, 90 to 150, 90 to 180, 90 to 210, 90 to 240, 90 to 270, 90 to 300, 90 to 330, 90 to 365, 120 to 150, 120 to 180, 120 to 210, 120 to 240, 120 to 270, 120 to 300, 120 to 330, 120 to 365, 150 to 180, 150 to 210, 150 to 240, 150 to 270, 150 to 300, 150 to 330, 150 to 365, 180 to 210, 180 to 240, 180 to 270, 180 to 300, 180 to 330, 180 to 365, 210 to 240, 210 to 270, 210 to 300, 210 to 330, 210 to 365, 240 to 270, 240 to 300, 240 to 330, 240 to 365, 270 to 300, 270 to 330, 270 to 365, 300 to 330, 300 to 365, or 330 to 365. In some embodiments, (n) is 14, 30, 60, 90, 120, 150, 180, 210, 240, 270, 300, 330, or 365.

Provided herein are methods, comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods, comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods, comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

In some embodiments, the method is a method of treating inflammation (e.g., Fas-mediated inflammation) in an eye (e.g., the retinal tissue of an eye). In some embodiments, the method is a method of inhibiting, reducing, and/or preventing inflammation (e.g., Fas-mediated inflammation) in an eye (e.g., the retinal tissue of an eye). In some embodiments, the method includes treating symptoms associated with inflammation in the eye (e.g., increased intraocular pressure, loss of and/or decrease in visual acuity, blurred vision, distorted vision, etc.). In some embodiments, retinal inflammation can be determined by observing the symptoms associated with inflammation in the eye (e.g., retinal degeneration, increased intraocular pressure, loss of and/or decrease in visual acuity, blurred vision, distorted vision, etc.) and/or by a biological assay detecting the presence of inflammatory molecules (e.g., inflammatory cytokines) in a sample (e.g., vitreous humor sample) taken from the eye.

In some embodiments, the method is a method of treating retinal cell loss (e.g., retinal pigment epithelial cells, retinal ganglion cells, and/or photoreceptors) in an eye (e.g., the retinal tissue of an eye). In some embodiments, the method is a method of inhibiting, reducing, and/or preventing retinal cell loss (e.g., retinal pigment epithelial cells, retinal ganglion cells, and/or photoreceptors) in an eye (e.g., the retinal tissue of an eye). In some embodiments, the method includes treating symptoms associated with retinal cell loss (e.g., retinal pigment epithelial cells, retinal ganglion cells, and/or photoreceptors) in the eye (e.g., retinal degeneration, increased intraocular pressure, loss of and/or decrease in visual acuity, blurred vision, distorted vision, etc.).

Provided herein are methods of treating photoreceptor cell death (or a symptom thereof), comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating photoreceptor cell death (or a symptom thereof), comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating photoreceptor cell death (or a symptom thereof), comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein are methods of treating photoreceptor cell death (or a symptom thereof), comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating photoreceptor cell death (or a symptom thereof), comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating photoreceptor cell death (or a symptom thereof), comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein are methods of treating photoreceptor cell death (or a symptom thereof), comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating photoreceptor cell death (or a symptom thereof), comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating photoreceptor cell death (or a symptom thereof), comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein are methods of treating photoreceptor cell death (or a symptom thereof), comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating photoreceptor cell death (or a symptom thereof), comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating photoreceptor cell death (or a symptom thereof), comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating photoreceptor cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein are methods of treating retinal cell death (or a symptom thereof), comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating retinal cell death (or a symptom thereof), comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating retinal cell death (or a symptom thereof), comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating retinal cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating retinal cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating retinal cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating retinal cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating retinal cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating retinal cell death (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

In some embodiments, the method is a method of treating retinal degeneration. In some embodiments, the method is a method of inhibiting, reducing, and/or preventing retinal degeneration. In some embodiments, the method includes treating symptoms associated with retinal degeneration (e.g., increased intraocular pressure, loss of and/or decrease in visual acuity, blurred vision, distorted vision, etc.). In some embodiments, retinal degeneration comprises the loss and/or death of retinal pigment epithelial cells, retinal ganglion cells, photoreceptors, or a combination thereof. In some embodiments, retinal degeneration comprises the loss and/or death of retinal pigment epithelial cells. In some embodiments, retinal degeneration comprises the loss and/or death of retinal ganglion cells. In some embodiments, retinal degeneration comprises the loss and/or death of photoreceptors. In some embodiments, retinal degeneration comprises the loss and/or death a combination of retinal pigment epithelial cells, retinal ganglion cells, and/or photoreceptors.

Provided herein are methods of treating retinal degeneration (or a symptom thereof), comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating retinal degeneration (or a symptom thereof), comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating retinal degeneration (or a symptom thereof), comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating retinal degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating retinal degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating retinal degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating retinal degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating retinal degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating retinal degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

In some embodiments, the method is a method of treating an ocular disease, disorder, or condition. In some embodiments, the method is a method of inhibiting, reducing, and/or preventing an ocular disease, disorder, or condition. In some embodiments, the method includes treating symptoms associated with an ocular disease, disorder, or condition (e.g., retinal degeneration, increased intraocular pressure, loss of and/or decrease in visual acuity, blurred vision, distorted vision, etc.). In some embodiments, the ocular disease, disorder, or condition is a chronic ocular disease, disorder, or condition. In some embodiments, the ocular disease, disorder, or condition comprises the loss and/or death of retinal pigment epithelial cells, retinal ganglion cells, photoreceptors, or a combination thereof. In some embodiments, the ocular disease, disorder, or condition comprises the loss and/or death of retinal pigment epithelial cells. In some embodiments, the ocular disease, disorder, or condition comprises the loss and/or death of retinal ganglion cells. In some embodiments, the ocular disease, disorder, or condition comprises the loss and/or death of photoreceptors. In some embodiments, the ocular disease, disorder, or condition comprises the loss and/or death a combination of retinal pigment epithelial cells, retinal ganglion cells, and/or photoreceptors.

Provided herein are methods of treating an ocular disease, disorder, or condition (or a symptom thereof), comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating an ocular disease, disorder, or condition (or a symptom thereof), comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating an ocular disease, disorder, or condition (or a symptom thereof), comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating an ocular disease, disorder, or condition (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating an ocular disease, disorder, or condition (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating an ocular disease, disorder, or condition (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating an ocular disease, disorder, or condition (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating an ocular disease, disorder, or condition (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating an ocular disease, disorder, or condition (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

In some embodiments, the method is a method of treating glaucoma. In some embodiments, the method is a method of inhibiting, reducing, and/or preventing glaucoma. In some embodiments, the method includes treating symptoms associated with glaucoma (e.g., retinal degeneration, increased intraocular pressure, loss of and/or decrease in visual acuity, blurred vision, distorted vision, etc.). In some embodiments, the ocular disease, disorder, or condition comprises the loss and/or death of retinal ganglion cells.

Provided herein are methods of treating glaucoma (or a symptom thereof), comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating glaucoma (or a symptom thereof), comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating glaucoma (or a symptom thereof), comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating glaucoma (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating glaucoma (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating glaucoma (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating glaucoma (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating glaucoma (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating glaucoma (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

In some embodiments, the ocular disease, disorder, or condition comprises macular degeneration. In some embodiments, the method is a method of inhibiting, reducing, and/or preventing macular degeneration. In some embodiments, the method includes treating symptoms associated with macular degeneration (e.g., retinal degeneration, increased intraocular pressure, loss of and/or decrease in visual acuity, blurred vision, distorted vision, etc.). In some embodiments, the macular degeneration is age-related macular degeneration, non-exudative age-related macular degeneration, or exudative age-related macular degeneration. In some embodiments, the macular degeneration is age-related macular degeneration. In some embodiments, the macular degeneration is non-exudative age-related macular degeneration. In some embodiments, the macular degeneration is exudative age-related macular degeneration. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors.

Provided herein are methods of treating macular degeneration (or a symptom thereof), comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating macular degeneration (or a symptom thereof), comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating macular degeneration (or a symptom thereof), comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating macular degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating macular degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating macular degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating macular degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating macular degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating macular degeneration (or a symptom thereof) comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

In some embodiments, the ocular disease, disorder, or condition comprises retinal detachment. In some embodiments, the method is a method of inhibiting, reducing, and/or preventing retinal detachment. In some embodiments, the method includes treating symptoms associated retinal detachment (e.g., retinal degeneration, increased intraocular pressure, loss of and/or decrease in visual acuity, blurred vision, distorted vision, etc.). In some embodiments, the retinal detachment is chronic retinal detachment. In some embodiments, retinal detachment comprises the loss and/or death of retinal pigment epithelial cells, retinal ganglion cells, photoreceptors, or a combination thereof. In some embodiments, retinal detachment comprises the loss and/or death of retinal pigment epithelial cells. In some embodiments, retinal detachment comprises the loss and/or death of retinal ganglion cells. In some embodiments, retinal detachment comprises the loss and/or death of photoreceptors. In some embodiments, retinal detachment comprises the loss and/or death a combination of retinal pigment epithelial cells, retinal ganglion cells, and/or photoreceptors.

In some embodiments, the method is a method of treating a loss in visual acuity. In some embodiments, the ocular disease, disorder, or condition comprises a loss in visual acuity. In some embodiments, a loss in visual acuity is characterized by and/or equivalent to a reduction in visual acuity (e.g., a reduction from 20/100 to 20/200, or a reduction from 20/40 to 20/50, etc.). In some embodiments, the method is a method of treating ocular hypertension (e.g., elevated intraocular pressure). In some embodiments, the method is a method of treating ocular hypertension (e.g., elevated intraocular pressure). In some embodiments, ocular hypertension is characterized by an intraocular pressures of 21 mm Hg or greater. In some embodiments, ocular hypertension includes by an intraocular pressures of 18 mm Hg or greater.

In some embodiments, the method is a method of treating a loss in visual function. In some embodiments, the ocular disease, disorder, or condition comprises a loss in visual function. In some embodiments, a loss in visual function is characterized by and/or equivalent to a reduction in visual function assessments (e.g., visual acuity, field of vision, contrast sensitivity, binocular function, and/or light/dark adaptation).

Provided herein are methods of treating a loss in visual acuity and/or visual function, comprising: administering plurality of compositions comprising a peptide comprising an amino acid sequence HHIYLGAVNYIY or a variant sequence thereof, or a pharmaceutically-acceptable salt of the peptide, whereby each of the plurality of compositions is administered to the to a vitreous humor of an eye, thereby providing the peptide to retinal tissue in the eye, whereby the peptide has a half-life in the vitreous humor of at least about (z) days. Also provided are methods of treating a loss in visual acuity and/or visual function, comprising: administering plurality of compositions comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days. Further provided are methods of treating a loss in visual acuity and/or visual function, comprising: administering plurality of composition comprising a peptide having the structure of Formula I or a pharmaceutically-acceptable salt thereof, whereby each of the plurality of compositions is administered to the to a vitreous humor of an eye, thereby providing the peptide to retinal tissue in the eye, whereby the peptide has a half-life in the vitreous humor of at least about (z) days.

Provided herein is the use of a composition comprising a peptide comprising an amino acid sequence HHIYLGAVNYIY or a variant sequence thereof, or a pharmaceutically-acceptable salt of the peptide in a method of treating a loss in visual acuity and/or visual function comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is the use of a composition comprising a peptide a peptide having the structure of Formula I or a pharmaceutically-acceptable salt thereof in a method of treating a loss in visual acuity and/or visual function comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is the use of a composition comprising a peptide a peptide having the structure of Formula III or a pharmaceutically-acceptable salt thereof in a method of treating a loss in visual acuity and/or visual function comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

Provided herein is a composition comprising a peptide comprising an amino acid sequence HHIYLGAVNYIY or a variant sequence thereof, or a pharmaceutically-acceptable salt of the peptide for use in a method of treating a loss in visual acuity and/or visual function comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Also provided is a composition comprising a peptide a peptide having the structure of Formula I or a pharmaceutically-acceptable salt thereof for use in a method of treating a loss in visual acuity and/or visual function comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days. Further provided herein is a composition comprising a peptide a peptide having the structure of Formula III or a pharmaceutically-acceptable salt thereof for use in a method of treating a loss in visual acuity and/or visual function comprising: administering a plurality of compositions, whereby each of the plurality of compositions is administered to the to a vitreous humor of an eye, thereby providing the peptide to retinal tissue in the eye, whereby the peptide has a half-life in the vitreous humor of at least about (z) days.

In some embodiments, (z) is 14 to 270. In some embodiments, (z) is 30 to 270. In some embodiments, it is not feasible to sample the vitreous humor of human eye. In such instances, the half-life can therefore be analogous to, measured in, and/or extrapolated from the (z) of a pig eye or a rabbit eye or monkey eye. Accordingly, in some embodiments, the peptide has a half-life in the vitreous humor of a pig eye of at least about (z) days. In some embodiments, the peptide has a half-life in the vitreous humor of a rabbit eye of at least about (z) days. In some embodiments, (z) is 30 to 270. In some embodiments, (z) is at least 14. In some embodiments, (z) is at least 30. In some embodiments, (z) is at least 60. In some embodiments, (z) is at least 90. In some embodiments, (z) is at least 120. In some embodiments, (z) is at least 150. In some embodiments, (z) is at least 180. In some embodiments, (z) is at least 210. In some embodiments, (z) is at least 240. In some embodiments, (z) is at least 270. In some embodiments, (z) is greater than 30. In some embodiments, (z) is greater than 60. In some embodiments, (z) is greater than 90. In some embodiments, (z) is greater than 120. In some embodiments, (z) is greater than 150. In some embodiments, (z) is greater than 180. In some embodiments, (z) is greater than 210. In some embodiments, (z) is greater than 240. In some embodiments, (z) is greater than 270. In some embodiments, (z) is 30 to 60, 30 to 90, 30 to 120, 30 to 150, 30 to 180, 30 to 210, 30 to 240, 30 to 270, 60 to 90, 60 to 120, 60 to 150, 60 to 180, 60 to 210, 60 to 240, 60 to 270, 90 to 120, 90 to 150, 90 to 180, 90 to 210, 90 to 240, 90 to 270, 120 to 150, 120 to 180, 120 to 210, 120 to 240, 120 to 270, 150 to 180, 150 to 210, 150 to 240, 150 to 270, 180 to 210, 180 to 240, 180 to 270, 210 to 240, 210 to 270, or 240 to 270. In some embodiments, (z) is 14, 30, 60, 90, 120, 150, 180, 210, 240, or 270.

Provided herein are methods, comprising: (a) administering a first composition comprising a peptide comprising an amino acid sequence HHIYLGAVNYIY or a variant sequence thereof, or a pharmaceutically-acceptable salt of the peptide, to a vitreous humor of an eye; and (b) administering at least a second composition comprising the peptide to the vitreous humor of the eye, and wherein the at least second dose is administered no less than about (y) days after the first administration. Also provided are methods, comprising: (a) administering a first composition comprising a peptide having the structure of Formula I or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. Further proved are methods, comprising: (a) administering a first composition comprising a peptide having the structure of Formula III or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. In some embodiments, the method comprises (c) administering at least a second composition comprising the peptide to the vitreous humor of the eye, and wherein the at least second dose is administered no less than about (y) days after the second dose. Provided herein is the use of a composition comprising a peptide comprising an amino acid sequence HHIYLGAVNYIY or a variant sequence thereof, or a pharmaceutically-acceptable salt of the peptide, in a method comprising: (a) administering a first composition comprising to a vitreous humor of an eye; and (b) administering at least a second composition comprising the peptide to the vitreous humor of the eye, and wherein the at least second dose is administered no less than about (y) days after the first administration. Provided herein is the use of a composition comprising a peptide comprising an amino acid sequence HHIYLGAVNYIY or a variant sequence thereof, or a pharmaceutically-acceptable salt of the peptide, in a method comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is the use of a composition comprising a peptide comprising having the structure of Formula I, or a pharmaceutically-acceptable salt thereof, in a method comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is the use of a composition comprising a peptide comprising having the structure of Formula III, or a pharmaceutically-acceptable salt thereof, in a method comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein is a composition comprising a peptide comprising an amino acid sequence HHIYLGAVNYIY or a variant sequence thereof, or a pharmaceutically-acceptable salt of the peptide, for use in a method comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is a composition comprising a peptide comprising having the structure of Formula I, or a pharmaceutically-acceptable salt thereof, for use in a method comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is a composition comprising a peptide comprising having the structure of Formula III, or a pharmaceutically-acceptable salt thereof, for use in a method comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein are methods, comprising: administering a plurality of compositions to a vitreous humor of an eye (e.g., over a plurality of administrations), the plurality of compositions being administered no more frequently than once every about (y) days, and each of the plurality of compositions comprising a peptide comprising an amino acid sequence HHIYLGAVNYIY or variant sequence thereof, or a pharmaceutically-acceptable salt of the peptide. Also provided are methods, comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula I, or a pharmaceutically-acceptable salt thereof. Further provided are methods, comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula III, or a pharmaceutically-acceptable salt thereof. Provided herein is the use of a compositions comprising a peptide comprising an amino acid sequence HHIYLGAVNYIY or variant sequence thereof, or a pharmaceutically-acceptable salt of the peptide, in a method comprising: administering a plurality of compositions to a vitreous humor of an eye (e.g., over a plurality of administrations), the plurality of compositions being administered no more frequently than once every about (y) days. Also provided herein is the use of a compositions comprising a peptide having the structure of Formula III, or a pharmaceutically-acceptable salt thereof, in a method comprising: administering a plurality of compositions to a vitreous humor of an eye (e.g., over a plurality of administrations), the plurality of compositions being administered no more frequently than once every about (y) days. Further provided herein is the use of a compositions comprising a peptide having the structure of Formula I, or a pharmaceutically-acceptable salt thereof, in a method comprising: administering a plurality of compositions to a vitreous humor of an eye (e.g., over a plurality of administrations), the plurality of compositions being administered no more frequently than once every about (y) days. Provided herein are compositions comprising a peptide comprising an amino acid sequence HHIYLGAVNYIY or variant sequence thereof, or a pharmaceutically-acceptable salt of the peptide, for use in a method comprising: administering a plurality of compositions to a vitreous humor of an eye (e.g., over a plurality of administrations), the plurality of compositions being administered no more frequently than once every about (y) days. Provided herein are compositions comprising a peptide having the structure of Formula III, or a pharmaceutically-acceptable salt thereof, for use in a method comprising: administering a plurality of compositions to a vitreous humor of an eye (e.g., over a plurality of administrations), the plurality of compositions being administered no more frequently than once every about (y) days. Provided herein are compositions comprising a peptide having the structure of Formula I, or a pharmaceutically-acceptable salt thereof, for use in a method comprising: administering a plurality of compositions to a vitreous humor of an eye (e.g., over a plurality of administrations), the plurality of compositions being administered no more frequently than once every about (y) days.

In some embodiments, the method is a method of treating inflammation (e.g., Fas-mediated inflammation) in an eye (e.g., the retinal tissue of an eye). In some embodiments, the method is a method of inhibiting, reducing, and/or preventing inflammation (e.g., Fas-mediated inflammation) in an eye (e.g., the retinal tissue of an eye). In some embodiments, the method includes treating symptoms associated with inflammation in the eye (e.g., increased intraocular pressure, loss of and/or decrease in visual acuity, blurred vision, distorted vision, etc.). In some embodiments, retinal inflammation can be determined by observing the symptoms associated with inflammation in the eye (e.g., retinal degeneration, increased intraocular pressure, loss of and/or decrease in visual acuity, blurred vision, distorted vision, etc.) and/or by a biological assay detecting the presence of inflammatory molecules (e.g., inflammatory cytokines) in a sample (e.g., vitreous humor sample) taken from the eye.

In some embodiments, the method is a method of treating retinal cell loss (e.g., retinal pigment epithelial cells, retinal ganglion cells, and/or photoreceptors) in an eye (e.g., the retinal tissue of an eye). In some embodiments, the method is a method of inhibiting, reducing, and/or preventing retinal cell loss (e.g., retinal pigment epithelial cells, retinal ganglion cells, and/or photoreceptors) in an eye (e.g., the retinal tissue of an eye). In some embodiments, the method includes treating symptoms associated with retinal cell loss (e.g., retinal pigment epithelial cells, retinal ganglion cells, and/or photoreceptors) in the eye (e.g., retinal degeneration, increased intraocular pressure, loss of and/or decrease in visual acuity, blurred vision, distorted vision, etc.).

Provided herein are methods of treating retinal degeneration (or a symptom thereof), comprising: (a) administering a first composition comprising a peptide comprising an amino acid sequence HHIYLGAVNYIY or a variant sequence thereof, or a pharmaceutically-acceptable salt of the peptide, to a vitreous humor of an eye; and (b) administering at least a second composition comprising the peptide to the vitreous humor of the eye, and wherein the at least second dose is administered no less than about (y) days after the first administration. Also provided are methods of treating retinal degeneration (or a symptom thereof), comprising: (a) administering a first composition comprising a peptide having the structure of Formula I or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. Further proved are methods of treating retinal degeneration (or a symptom thereof), comprising: (a) administering a first composition comprising a peptide having the structure of Formula III or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. In some embodiments, the method comprises (c) administering at least a second composition comprising the peptide to the vitreous humor of the eye, and wherein the at least second dose is administered no less than about (y) days after the second dose.

Provided herein is the use of a composition comprising a peptide comprising an amino acid sequence HHIYLGAVNYIY or a variant sequence thereof, or a pharmaceutically-acceptable salt of the peptide, in a method of treating retinal degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Provided herein is the use of a composition comprising a peptide comprising an amino acid sequence HHIYLGAVNYIY or a variant sequence thereof, or a pharmaceutically-acceptable salt of the peptide, in a method of treating retinal degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is the use of a composition comprising a peptide comprising having the structure of Formula I, or a pharmaceutically-acceptable salt thereof, in a method of treating retinal degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is the use of a composition comprising a peptide comprising having the structure of Formula III, or a pharmaceutically-acceptable salt thereof, in a method of treating retinal degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein is a composition comprising a peptide comprising an amino acid sequence HHIYLGAVNYIY or a variant sequence thereof, or a pharmaceutically-acceptable salt of the peptide, for use in a method of treating retinal degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is a composition comprising a peptide comprising having the structure of Formula I, or a pharmaceutically-acceptable salt thereof, for use in a method of treating retinal degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is a composition comprising a peptide comprising having the structure of Formula III, or a pharmaceutically-acceptable salt thereof, for use in a method of treating retinal degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein are methods of treating retinal degeneration (or a symptom thereof), comprising: administering a plurality of compositions to a vitreous humor of an eye (e.g. over a plurality of administrations), the plurality of compositions being administered no more frequently than once every about (y) days, and each of the plurality of compositions comprising a peptide comprising an amino acid sequence HHIYLGAVNYIY or variant sequence thereof, or a pharmaceutically-acceptable salt of the peptide. Also provided are methods of treating retinal degeneration (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula I, or a pharmaceutically-acceptable salt thereof. Further provided are methods of treating retinal degeneration (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula III, or a pharmaceutically-acceptable salt thereof.

Provided herein is the use of a compositions comprising a peptide comprising an amino acid sequence HHIYLGAVNYIY or variant sequence thereof, or a pharmaceutically-acceptable salt of the peptide, in a method of treating retinal degeneration (or a symptom thereof) comprising: administering a plurality of compositions to a vitreous humor of an eye (e.g., over a plurality of administrations), the plurality of compositions being administered no more frequently than once every about (y) days. Also provided herein is the use of a compositions comprising a peptide having the structure of Formula I, or a pharmaceutically-acceptable salt thereof, in a method of treating retinal degeneration (or a symptom thereof) comprising: administering a plurality of compositions to a vitreous humor of an eye (e.g., over a plurality of administrations), the plurality of compositions being administered no more frequently than once every about (y) days. Further provided herein is the use of a compositions comprising a peptide having the structure of Formula III, or a pharmaceutically-acceptable salt thereof, in a method of treating retinal degeneration (or a symptom thereof) comprising: administering a plurality of compositions to a vitreous humor of an eye (e.g., over a plurality of administrations), the plurality of compositions being administered no more frequently than once every about (y) days.

Provided herein are compositions comprising a peptide comprising an amino acid sequence HHIYLGAVNYIY or variant sequence thereof, or a pharmaceutically-acceptable salt of the peptide, for use in a method of treating retinal degeneration (or a symptom thereof) comprising: administering a plurality of compositions to a vitreous humor of an eye (e.g., over a plurality of administrations), the plurality of compositions being administered no more frequently than once every about (y) days. Provided herein are compositions comprising a peptide having the structure of Formula I, or a pharmaceutically-acceptable salt thereof, for use in a method of treating retinal degeneration (or a symptom thereof) comprising: administering a plurality of compositions to a vitreous humor of an eye (e.g., over a plurality of administrations), the plurality of compositions being administered no more frequently than once every about (y) days. Provided herein are compositions comprising a peptide having the structure of Formula III, or a pharmaceutically-acceptable salt thereof, for use in a method of treating retinal degeneration (or a symptom thereof) comprising: administering a plurality of compositions to a vitreous humor of an eye (e.g., over a plurality of administrations), the plurality of compositions being administered no more frequently than once every about (y) days.

In some embodiments, the method is a method of treating retinal degeneration. In some embodiments, the method is a method of inhibiting, reducing, and/or preventing retinal degeneration. In some embodiments, the method includes treating symptoms associated with retinal degeneration (e.g., increased intraocular pressure, loss of and/or decrease in visual acuity, blurred vision, distorted vision, etc.). In some embodiments, retinal degeneration comprises the loss and/or death of retinal pigment epithelial cells, retinal ganglion cells, photoreceptors, or a combination thereof. In some embodiments, retinal degeneration comprises the loss and/or death of retinal pigment epithelial cells. In some embodiments, retinal degeneration comprises the loss and/or death of retinal ganglion cells. In some embodiments, retinal degeneration comprises the loss and/or death of photoreceptors. In some embodiments, retinal degeneration comprises the loss and/or death a combination of retinal pigment epithelial cells, retinal ganglion cells, and/or photoreceptors.

In some embodiments, the method is a method of treating an ocular disease, disorder, or condition. In some embodiments, the method is a method of inhibiting, reducing, and/or preventing an ocular disease, disorder, or condition. In some embodiments, the method includes treating symptoms associated with an ocular disease, disorder, or condition (e.g., retinal degeneration, increased intraocular pressure, loss of and/or decrease in visual acuity, blurred vision, distorted vision, etc.). In some embodiments, the ocular disease, disorder, or condition is a chronic ocular disease, disorder, or condition. In some embodiments, the ocular disease, disorder, or condition comprises the loss and/or death of retinal pigment epithelial cells, retinal ganglion cells, photoreceptors, or a combination thereof. In some embodiments, the ocular disease, disorder, or condition comprises the loss and/or death of retinal pigment epithelial cells. In some embodiments, the ocular disease, disorder, or condition comprises the loss and/or death of retinal ganglion cells. In some embodiments, the ocular disease, disorder, or condition comprises the loss and/or death of photoreceptors. In some embodiments, the ocular disease, disorder, or condition comprises the loss and/or death a combination of retinal pigment epithelial cells, retinal ganglion cells, and/or photoreceptors.

Provided herein are methods of treating an ocular disease, disorder, and/or condition (or a symptom thereof), comprising: (a) administering a first composition comprising a peptide comprising an amino acid sequence HHIYLGAVNYIY or a variant sequence thereof, or a pharmaceutically-acceptable salt of the peptide, to a vitreous humor of an eye; and (b) administering at least a second composition comprising the peptide to the vitreous humor of the eye, and wherein the at least second dose is administered no less than about (y) days after the first administration. Also provided are methods of treating an ocular disease, disorder, and/or condition (or a symptom thereof), comprising: (a) administering a first composition comprising a peptide having the structure of Formula I or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. Further proved are methods of treating an ocular disease, disorder, and/or condition (or a symptom thereof), comprising: (a) administering a first composition comprising a peptide having the structure of Formula III or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. In some embodiments, the method comprises (c) administering at least a second composition comprising the peptide to the vitreous humor of the eye, and wherein the at least second dose is administered no less than about (y) days after the second dose.

Provided herein is the use of a composition comprising a peptide comprising an amino acid sequence HHIYLGAVNYIY or a variant sequence thereof, or a pharmaceutically-acceptable salt of the peptide, in a method of treating an ocular disease, disorder, and/or condition (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Provided herein is the use of a composition comprising a peptide comprising an amino acid sequence HHIYLGAVNYIY or a variant sequence thereof, or a pharmaceutically-acceptable salt of the peptide, in a method of treating an ocular disease, disorder, and/or condition (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is the use of a composition comprising a peptide comprising having the structure of Formula I, or a pharmaceutically-acceptable salt thereof, in a method of treating an ocular disease, disorder, and/or condition (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is the use of a composition comprising a peptide comprising having the structure of Formula III, or a pharmaceutically-acceptable salt thereof, in a method of treating an ocular disease, disorder, and/or condition (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein is a composition comprising a peptide comprising an amino acid sequence HHIYLGAVNYIY or a variant sequence thereof, or a pharmaceutically-acceptable salt of the peptide, for use in a method of treating an ocular disease, disorder, and/or condition (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is a composition comprising a peptide comprising having the structure of Formula I, or a pharmaceutically-acceptable salt thereof, for use in a method of treating an ocular disease, disorder, and/or condition (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is a composition comprising a peptide comprising having the structure of Formula III, or a pharmaceutically-acceptable salt thereof, for use in a method of treating an ocular disease, disorder, and/or condition (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein are methods of treating an ocular disease, disorder, and/or condition (or a symptom thereof), comprising: administering a plurality of compositions to a vitreous humor of an eye (e.g. over a plurality of administrations), the plurality of compositions being administered no more frequently than once every about (y) days, and each of the plurality of compositions comprising a peptide comprising an amino acid sequence HHIYLGAVNYIY or variant sequence thereof, or a pharmaceutically-acceptable salt of the peptide. Also provided are methods of treating an ocular disease, disorder, and/or condition (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula I, or a pharmaceutically-acceptable salt thereof. Further provided are methods of treating an ocular disease, disorder, and/or condition (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula III, or a pharmaceutically-acceptable salt thereof.

Provided herein is the use of a compositions comprising a peptide comprising an amino acid sequence HHIYLGAVNYIY or variant sequence thereof, or a pharmaceutically-acceptable salt of the peptide, in a method of treating an ocular disease, disorder, and/or condition (or a symptom thereof) comprising: administering a plurality of compositions to a vitreous humor of an eye (e.g. over a plurality of administrations), the plurality of compositions being administered no more frequently than once every about (y) days. Also provided herein is the use of a compositions comprising a peptide having the structure of Formula I, or a pharmaceutically-acceptable salt thereof, in a method of treating an ocular disease, disorder, and/or condition (or a symptom thereof) comprising: administering a plurality of compositions to a vitreous humor of an eye (e.g., over a plurality of administrations), the plurality of compositions being administered no more frequently than once every about (y) days. Further provided herein is the use of a compositions comprising a peptide having the structure of Formula III, or a pharmaceutically-acceptable salt thereof, in a method of treating an ocular disease, disorder, and/or condition (or a symptom thereof) comprising: administering a plurality of compositions to a vitreous humor of an eye (e.g., over a plurality of administrations), the plurality of compositions being administered no more frequently than once every about (y) days.

Provided herein are compositions comprising a peptide comprising an amino acid sequence HHIYLGAVNYIY or variant sequence thereof, or a pharmaceutically-acceptable salt of the peptide, for use in a method of treating an ocular disease, disorder, and/or condition (or a symptom thereof) comprising: administering a plurality of compositions to a vitreous humor of an eye (e.g. over a plurality of administrations), the plurality of compositions being administered no more frequently than once every about (y) days. Provided herein are compositions comprising a peptide having the structure of Formula I, or a pharmaceutically-acceptable salt thereof, for use in a method of treating an ocular disease, disorder, and/or condition (or a symptom thereof) comprising: administering a plurality of compositions to a vitreous humor of an eye (e.g., over a plurality of administrations), the plurality of compositions being administered no more frequently than once every about (y) days. Provided herein are compositions comprising a peptide having the structure of Formula III, or a pharmaceutically-acceptable salt thereof, for use in a method of treating an ocular disease, disorder, and/or condition (or a symptom thereof) comprising: administering a plurality of compositions to a vitreous humor of an eye (e.g., over a plurality of administrations), the plurality of compositions being administered no more frequently than once every about (y) days.

In some embodiments, the method is a method of treating glaucoma. In some embodiments, the method is a method of inhibiting, reducing, and/or preventing glaucoma. In some embodiments, the method includes treating symptoms associated with glaucoma (e.g., retinal degeneration, increased intraocular pressure, loss of and/or decrease in visual acuity, blurred vision, distorted vision, etc.). In some embodiments, the ocular disease, disorder, or condition comprises the loss and/or death of retinal ganglion cells.

Provided herein are methods of treating glaucoma (or a symptom thereof), comprising: (a) administering a first composition comprising a peptide comprising an amino acid sequence HHIYLGAVNYIY or a variant sequence thereof, or a pharmaceutically-acceptable salt of the peptide, to a vitreous humor of an eye; and (b) administering at least a second composition comprising the peptide to the vitreous humor of the eye, and wherein the at least second dose is administered no less than about (y) days after the first administration. Also provided are methods of treating glaucoma (or a symptom thereof), comprising: (a) administering a first composition comprising a peptide having the structure of Formula I or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. Further proved are methods of treating glaucoma (or a symptom thereof), comprising: (a) administering a first composition comprising a peptide having the structure of Formula III or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. In some embodiments, the method comprises (c) administering at least a second composition comprising the peptide to the vitreous humor of the eye, and wherein the at least second dose is administered no less than about (y) days after the second dose.

Provided herein is the use of a composition comprising a peptide comprising an amino acid sequence HHIYLGAVNYIY or a variant sequence thereof, or a pharmaceutically-acceptable salt of the peptide, in a method of treating glaucoma (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Provided herein is the use of a composition comprising a peptide comprising an amino acid sequence HHIYLGAVNYIY or a variant sequence thereof, or a pharmaceutically-acceptable salt of the peptide, in a method of treating glaucoma (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is the use of a composition comprising a peptide comprising having the structure of Formula I, or a pharmaceutically-acceptable salt thereof, in a method of treating glaucoma (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is the use of a composition comprising a peptide comprising having the structure of Formula III, or a pharmaceutically-acceptable salt thereof, in a method of treating glaucoma (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein is a composition comprising a peptide comprising an amino acid sequence HHIYLGAVNYIY or a variant sequence thereof, or a pharmaceutically-acceptable salt of the peptide, for use in a method of treating glaucoma (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is a composition comprising a peptide comprising having the structure of Formula I, or a pharmaceutically-acceptable salt thereof, for use in a method of treating glaucoma (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is a composition comprising a peptide comprising having the structure of Formula III, or a pharmaceutically-acceptable salt thereof, for use in a method of treating glaucoma (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein are methods of treating glaucoma (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide comprising an amino acid sequence HHIYLGAVNYIY or variant sequence thereof, or a pharmaceutically-acceptable salt of the peptide. Also provided are methods of treating glaucoma (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula I, or a pharmaceutically-acceptable salt thereof. Further provided are methods of treating glaucoma (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula III, or a pharmaceutically-acceptable salt thereof.

Provided herein is the use of a compositions comprising a peptide comprising an amino acid sequence HHIYLGAVNYIY or variant sequence thereof, or a pharmaceutically-acceptable salt of the peptide, in a method of treating glaucoma (or a symptom thereof) comprising: administering a plurality of compositions to a vitreous humor of an eye (e.g., over a plurality of administrations), the plurality of compositions being administered no more frequently than once every about (y) days. Also provided herein is the use of a compositions comprising a peptide having the structure of Formula I, or a pharmaceutically-acceptable salt thereof, in a method of treating glaucoma (or a symptom thereof) comprising: administering a plurality of compositions to a vitreous humor of an eye (e.g., over a plurality of administrations), the plurality of compositions being administered no more frequently than once every about (y) days. Further provided herein is the use of a compositions comprising a peptide having the structure of Formula III, or a pharmaceutically-acceptable salt thereof, in a method of treating glaucoma (or a symptom thereof) comprising: administering a plurality of compositions to a vitreous humor of an eye (e.g., over a plurality of administrations), the plurality of compositions being administered no more frequently than once every about (y) days.

Provided herein is a compositions comprising a peptide comprising an amino acid sequence HHIYLGAVNYIY or variant sequence thereof, or a pharmaceutically-acceptable salt of the peptide, for use in a method of treating glaucoma (or a symptom thereof) comprising: administering a plurality of compositions to a vitreous humor of an eye (e.g., over a plurality of administrations), the plurality of compositions being administered no more frequently than once every about (y) days. Provided herein is a compositions comprising a peptide having the structure of Formula I, or a pharmaceutically-acceptable salt thereof, for use in a method of treating glaucoma (or a symptom thereof) comprising: administering a plurality of compositions to a vitreous humor of an eye (e.g., over a plurality of administrations), the plurality of compositions being administered no more frequently than once every about (y) days. Provided herein is a compositions comprising a peptide having the structure of Formula III, or a pharmaceutically-acceptable salt thereof, for use in a method of treating glaucoma (or a symptom thereof) comprising: administering a plurality of compositions to a vitreous humor of an eye (e.g., over a plurality of administrations), the plurality of compositions being administered no more frequently than once every about (y) days.

In some embodiments, the method is a method of treating macular degeneration. In some embodiments, the method is a method of inhibiting, reducing, and/or preventing macular degeneration. In some embodiments, the method includes treating symptoms associated with macular degeneration (e.g., retinal degeneration, increased intraocular pressure, loss of and/or decrease in visual acuity, blurred vision, distorted vision, etc.). In some embodiments, the macular degeneration is age-related macular degeneration, non-exudative age-related macular degeneration, or exudative age-related macular degeneration. In some embodiments, the macular degeneration is age-related macular degeneration. In some embodiments, the macular degeneration is non-exudative age-related macular degeneration. In some embodiments, the macular degeneration is exudative age-related macular degeneration. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors.

Provided herein are methods of treating macular degeneration (or a symptom thereof), comprising: (a) administering a first composition comprising a peptide comprising an amino acid sequence HHIYLGAVNYIY or a variant sequence thereof, or a pharmaceutically-acceptable salt of the peptide, to a vitreous humor of an eye; and (b) administering at least a second composition comprising the peptide to the vitreous humor of the eye, and wherein the at least second dose is administered no less than about (y) days after the first administration. Also provided are methods of treating macular degeneration (or a symptom thereof), comprising: (a) administering a first composition comprising a peptide having the structure of Formula I or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. Further proved are methods of treating macular degeneration (or a symptom thereof), comprising: (a) administering a first composition comprising a peptide having the structure of Formula III or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. In some embodiments, the method comprises (c) administering at least a second composition comprising the peptide to the vitreous humor of the eye, and wherein the at least second dose is administered no less than about (y) days after the second dose.

Provided herein is the use of a composition comprising a peptide comprising an amino acid sequence HHIYLGAVNYIY or a variant sequence thereof, or a pharmaceutically-acceptable salt of the peptide, in a method of treating macular degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Provided herein is the use of a composition comprising a peptide comprising an amino acid sequence HHIYLGAVNYIY or a variant sequence thereof, or a pharmaceutically-acceptable salt of the peptide, in a method of treating macular degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is the use of a composition comprising a peptide comprising having the structure of Formula I, or a pharmaceutically-acceptable salt thereof, in a method of treating macular degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is the use of a composition comprising a peptide comprising having the structure of Formula III, or a pharmaceutically-acceptable salt thereof, in a method of treating macular degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein is a composition comprising a peptide comprising an amino acid sequence HHIYLGAVNYIY or a variant sequence thereof, or a pharmaceutically-acceptable salt of the peptide, for use in a method of treating macular degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is a composition comprising a peptide comprising having the structure of Formula I, or a pharmaceutically-acceptable salt thereof, for use in a method of treating macular degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is a composition comprising a peptide comprising having the structure of Formula III, or a pharmaceutically-acceptable salt thereof, for use in a method of treating macular degeneration (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein are methods of treating macular degeneration (or a symptom thereof), comprising: administering a plurality of compositions to a vitreous humor of an eye (e.g. over a plurality of administrations), the plurality of compositions being administered no more frequently than once every about (y) days, and each of the plurality of compositions comprising a peptide comprising an amino acid sequence HHIYLGAVNYIY or variant sequence thereof, or a pharmaceutically-acceptable salt of the peptide. Also provided are methods of treating macular degeneration (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula I, or a pharmaceutically-acceptable salt thereof. Further provided are methods of treating macular degeneration (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula III, or a pharmaceutically-acceptable salt thereof.

Provided herein is the use of a compositions comprising a peptide comprising an amino acid sequence HHIYLGAVNYIY or variant sequence thereof, or a pharmaceutically-acceptable salt of the peptide, in a method of treating macular degeneration (or a symptom thereof) comprising: administering a plurality of compositions to a vitreous humor of an eye (e.g., over a plurality of administrations), the plurality of compositions being administered no more frequently than once every about (y) days. Also provided herein is the use of a compositions comprising a peptide having the structure of Formula I, or a pharmaceutically-acceptable salt thereof, in a method of treating macular degeneration (or a symptom thereof) comprising: administering a plurality of compositions to a vitreous humor of an eye (e.g., over a plurality of administrations), the plurality of compositions being administered no more frequently than once every about (y) days. Further provided herein is the use of a compositions comprising a peptide having the structure of Formula III, or a pharmaceutically-acceptable salt thereof, in a method of treating macular degeneration (or a symptom thereof) comprising: administering a plurality of compositions to a vitreous humor of an eye (e.g., over a plurality of administrations), the plurality of compositions being administered no more frequently than once every about (y) days.

Provided herein is a compositions comprising a peptide comprising an amino acid sequence HHIYLGAVNYIY or variant sequence thereof, or a pharmaceutically-acceptable salt of the peptide, for use in a method of treating macular degeneration (or a symptom thereof) comprising: administering a plurality of compositions to a vitreous humor of an eye (e.g., over a plurality of administrations), the plurality of compositions being administered no more frequently than once every about (y) days. Provided herein is a compositions comprising a peptide having the structure of Formula I, or a pharmaceutically-acceptable salt thereof, for use in a method of treating macular degeneration (or a symptom thereof) comprising: administering a plurality of compositions to a vitreous humor of an eye (e.g., over a plurality of administrations), the plurality of compositions being administered no more frequently than once every about (y) days. Provided herein is a compositions comprising a peptide having the structure of Formula III, or a pharmaceutically-acceptable salt thereof, for use in a method of treating macular degeneration (or a symptom thereof) comprising: administering a plurality of compositions to a vitreous humor of an eye (e.g., over a plurality of administrations), the plurality of compositions being administered no more frequently than once every about (y) days.

In some embodiments, the method is a method of treating retinal detachment. In some embodiments, the method is a method of inhibiting, reducing, and/or preventing retinal detachment. In some embodiments, the method includes treating symptoms associated retinal detachment (e.g., retinal degeneration, increased intraocular pressure, loss of and/or decrease in visual acuity, blurred vision, distorted vision, etc.). In some embodiments, the retinal detachment is chronic retinal detachment. In some embodiments, retinal detachment comprises the loss and/or death of retinal pigment epithelial cells, retinal ganglion cells, photoreceptors, or a combination thereof. In some embodiments, retinal detachment comprises the loss and/or death of retinal pigment epithelial cells. In some embodiments, retinal detachment comprises the loss and/or death of retinal ganglion cells. In some embodiments, retinal detachment comprises the loss and/or death of photoreceptors. In some embodiments, retinal detachment comprises the loss and/or death a combination of retinal pigment epithelial cells, retinal ganglion cells, and/or photoreceptors.

In some embodiments, the method is a method of treating a loss in visual acuity. In some embodiments, the method is a method of treating a loss in visual acuity. In some embodiments, a loss in visual acuity is characterized by and/or equivalent to a reduction in visual acuity (e.g., a reduction from 20/100 to 20/200, or a reduction from 20/40 to 20/50, etc.). In some embodiments, the method is a method of treating ocular hypertension (e.g., elevated intraocular pressure). In some embodiments, the method is a method of treating ocular hypertension (e.g., elevated intraocular pressure). In some embodiments, ocular hypertension is characterized by an intraocular pressures of 21 mm Hg or greater. In some embodiments, ocular hypertension includes by an intraocular pressures of 18 mm Hg or greater.

In some embodiments, the method is a method of treating a loss in visual function. In some embodiments, the method is a method of treating a loss in visual function. In some embodiments, a loss in visual function is characterized by and/or equivalent to a reduction in visual function assessments (e.g., visual acuity, field of vision, contrast sensitivity, binocular function, and/or light/dark adaptation).

Provided herein are methods of treating a reduction (e.g., loss) in visual acuity or function (or a symptom thereof), comprising: (a) administering a first composition comprising a peptide comprising an amino acid sequence HHIYLGAVNYIY or a variant sequence thereof, or a pharmaceutically-acceptable salt of the peptide, to a vitreous humor of an eye; and (b) administering at least a second composition comprising the peptide to the vitreous humor of the eye, and wherein the at least second dose is administered no less than about (y) days after the first administration. Also provided are methods of treating a reduction (e.g., loss) in visual acuity or function (or a symptom thereof), comprising: (a) administering a first composition comprising a peptide having the structure of Formula I or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. Further proved are methods of treating a reduction (e.g., loss) in visual acuity or function (or a symptom thereof), comprising: (a) administering a first composition comprising a peptide having the structure of Formula III or a pharmaceutically-acceptable salt thereof, to a vitreous humor of an eye; and (b) administering at least a second composition comprising the peptide to the vitreous humor of the eye, and wherein the at least second dose is administered no less than about (y) days after the first administration. In some embodiments, the method comprises (c) administering at least a second composition comprising the peptide to the vitreous humor of the eye, and wherein the at least second dose is administered no less than about (y) days after the second dose.

Provided herein is the use of a composition comprising a peptide comprising an amino acid sequence HHIYLGAVNYIY or a variant sequence thereof, or a pharmaceutically-acceptable salt of the peptide, in a method of treating a reduction (e.g., loss) in visual acuity or function (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Provided herein is the use of a composition comprising a peptide comprising an amino acid sequence HHIYLGAVNYIY or a variant sequence thereof, or a pharmaceutically-acceptable salt of the peptide, in a method of treating a reduction (e.g., loss) in visual acuity or function (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is the use of a composition comprising a peptide comprising having the structure of Formula I, or a pharmaceutically-acceptable salt thereof, in a method of treating a reduction (e.g., loss) in visual acuity or function (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is the use of a composition comprising a peptide comprising having the structure of Formula III, or a pharmaceutically-acceptable salt thereof, in a method of treating a reduction (e.g., loss) in visual acuity or function (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein is a composition comprising a peptide comprising an amino acid sequence HHIYLGAVNYIY or a variant sequence thereof, or a pharmaceutically-acceptable salt of the peptide, for use in a method of treating a reduction (e.g., loss) in visual acuity or function (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Also provided herein is a composition comprising a peptide comprising having the structure of Formula I, or a pharmaceutically-acceptable salt thereof, for use in a method of treating a reduction (e.g., loss) in visual acuity or function (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration. Further provided herein is a composition comprising a peptide comprising having the structure of Formula III, or a pharmaceutically-acceptable salt thereof, for use in a method of treating a reduction (e.g., loss) in visual acuity or function (or a symptom thereof) comprising: (a) administering a first composition comprising to a vitreous humor of an eye; and (b) administering at least a second composition comprising the peptide to the vitreous humor of the eye, and wherein the at least second dose is administered no less than about (y) days after the first administration.

Provided herein are methods of treating a reduction (e.g., loss) in visual acuity or function (or a symptom thereof), comprising: administering a plurality of compositions to a vitreous humor of an eye (e.g. over a plurality of administrations), the plurality of compositions being administered no more frequently than once every about (y) days, and each of the plurality of compositions comprising a peptide comprising an amino acid sequence HHIYLGAVNYIY or variant sequence thereof, or a pharmaceutically-acceptable salt of the peptide. Also provided are methods of treating a reduction (e.g., loss) in visual acuity or function (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula I, or a pharmaceutically-acceptable salt thereof. Further provided are methods of treating a reduction (e.g., loss) in visual acuity or function (or a symptom thereof), comprising: administering a plurality of compositions to a vitreous humor of an eye (e.g., over a plurality of administrations), the plurality of compositions being administered no more frequently than once every about (y) days, and each of the plurality of compositions comprising a peptide having the structure of Formula III, or a pharmaceutically-acceptable salt thereof.

Provided herein is the use of a compositions comprising a peptide comprising an amino acid sequence HHIYLGAVNYIY or variant sequence thereof, or a pharmaceutically-acceptable salt of the peptide, in a method of treating a reduction (e.g., loss) in visual acuity or function (or a symptom thereof) comprising: administering a plurality of compositions to a vitreous humor of an eye (e.g. over a plurality of administrations), the plurality of compositions being administered no more frequently than once every about (y) days. Also provided herein is the use of a compositions comprising a peptide having the structure of Formula I, or a pharmaceutically-acceptable salt thereof, in a method of treating a reduction (e.g., loss) in visual acuity or function (or a symptom thereof) comprising: administering a plurality of compositions to a vitreous humor of an eye (e.g., over a plurality of administrations), the plurality of compositions being administered no more frequently than once every about (y) days. Further provided herein is the use of a compositions comprising a peptide having the structure of Formula III, or a pharmaceutically-acceptable salt thereof, in a method of treating a reduction (e.g., loss) in visual acuity or function (or a symptom thereof) comprising: administering a plurality of compositions to a vitreous humor of an eye (e.g., over a plurality of administrations), the plurality of compositions being administered no more frequently than once every about (y) days.

Provided herein is a compositions comprising a peptide comprising an amino acid sequence HHIYLGAVNYIY or variant sequence thereof, or a pharmaceutically-acceptable salt of the peptide, for use in a method of treating a reduction (e.g., loss) in visual acuity or function (or a symptom thereof) comprising: administering a plurality of compositions to a vitreous humor of an eye (e.g. over a plurality of administrations), the plurality of compositions being administered no more frequently than once every about (y) days. Provided herein is a compositions comprising a peptide having the structure of Formula I, or a pharmaceutically-acceptable salt thereof, for use in a method of treating a reduction (e.g., loss) in visual acuity or function (or a symptom thereof) comprising: administering a plurality of compositions to a vitreous humor of an eye (e.g., over a plurality of administrations), the plurality of compositions being administered no more frequently than once every about (y) days. Provided herein is a compositions comprising a peptide having the structure of Formula III, or a pharmaceutically-acceptable salt thereof, for use in a method of treating a reduction (e.g., loss) in visual acuity or function (or a symptom thereof) comprising: administering a plurality of compositions to a vitreous humor of an eye (e.g., over a plurality of administrations), the plurality of compositions being administered no more frequently than once every about (y) days.

In some embodiments, (y) is 14 to 365. In some embodiments, (y) is 30 to 365. In some embodiments, (y) is 90 to 180. In some embodiments, (y) is at least 14. In some embodiments, (y) is at least 30. In some embodiments, (y) is at least 60. In some embodiments, (y) is at least 90. In some embodiments, (y) is at least 120. In some embodiments, (y) is at least 150. In some embodiments, (y) is at least 180. In some embodiments, (y) is at least 210. In some embodiments, (y) is at least 240. In some embodiments, (y) is at least 270. In some embodiments, (y) is at least 300. In some embodiments, (y) is at least 330. In some embodiments, (y) is at least 365. In some embodiments, (y) is greater than 30. In some embodiments, (y) is greater than 60. In some embodiments, (y) is greater than 90. In some embodiments, (y) is greater than 120. In some embodiments, (y) is greater than 150. In some embodiments, (y) is greater than 180. In some embodiments, (y) is greater than 210. In some embodiments, (y) is greater than 240. In some embodiments, (y) is greater than 270. In some embodiments, (y) is greater than 300. In some embodiments, (y) is greater than 330. In some embodiments, (y) is greater than 365. In some embodiments, (y) is 30 to 60, 30 to 90, 30 to 120, 30 to 150, 30 to 180, 30 to 210, 30 to 240, 30 to 270, 30 to 300, 30 to 330, 30 to 365, 60 to 90, 60 to 120, 60 to 150, 60 to 180, 60 to 210, 60 to 240, 60 to 270, 60 to 300, 60 to 330, 60 to 365, 90 to 120, 90 to 150, 90 to 180, 90 to 210, 90 to 240, 90 to 270, 90 to 300, 90 to 330, 90 to 365, 120 to 150, 120 to 180, 120 to 210, 120 to 240, 120 to 270, 120 to 300, 120 to 330, 120 to 365, 150 to 180, 150 to 210, 150 to 240, 150 to 270, 150 to 300, 150 to 330, 150 to 365, 180 to 210, 180 to 240, 180 to 270, 180 to 300, 180 to 330, 180 to 365, 210 to 240, 210 to 270, 210 to 300, 210 to 330, 210 to 365, 240 to 270, 240 to 300, 240 to 330, 240 to 365, 270 to 300, 270 to 330, 270 to 365, 300 to 330, 300 to 365, or 330 to 365. In some embodiments, (y) is 14, 30, 60, 90, 120, 150, 180, 210, 240, 270, 300, 330, or 365

In some embodiments, the method comprises using the vitreous humor as a depot to provide the peptide to retinal tissue in the eye, whereby the peptide is present in the vitreous humor for at (z) days. In some embodiments, (z) is 30 to 270. In some embodiments, it is not feasible to sample the vitreous humor of human eye. In such instances, the half-life can therefore be analogous to a pig eye or a rabbit eye. Accordingly, in some embodiments, the peptide has a half-life in the vitreous humor of a pig eye of at least about (z) days. In some embodiments, the peptide has a half-life in the vitreous humor of a rabbit eye of at least about (z) days. In some embodiments, (z) is 30 to 270. In some embodiments, (z) is at least 30. In some embodiments, (z) is at least 60. In some embodiments, (z) is at least 90. In some embodiments, (z) is at least 120. In some embodiments, (z) is at least 150. In some embodiments, (z) is at least 180. In some embodiments, (z) is at least 210. In some embodiments, (z) is at least 240. In some embodiments, (z) is at least 270. In some embodiments, (z) is greater than 30. In some embodiments, (z) is greater than 60. In some embodiments, (z) is greater than 90. In some embodiments, (z) is greater than 120. In some embodiments, (z) is greater than 150. In some embodiments, (z) is greater than 180. In some embodiments, (z) is greater than 210. In some embodiments, (z) is greater than 240. In some embodiments, (z) is greater than 270. In some embodiments, (z) is 30 to 60, 30 to 90, 30 to 120, 30 to 150, 30 to 180, 30 to 210, 30 to 240, 30 to 270, 60 to 90, 60 to 120, 60 to 150, 60 to 180, 60 to 210, 60 to 240, 60 to 270, 90 to 120, 90 to 150, 90 to 180, 90 to 210, 90 to 240, 90 to 270, 120 to 150, 120 to 180, 120 to 210, 120 to 240, 120 to 270, 150 to 180, 150 to 210, 150 to 240, 150 to 270, 180 to 210, 180 to 240, 180 to 270, 210 to 240, 210 to 270, or 240 to 270. In some embodiments, (z) is 30, 60, 90, 120, 150, 180, 210, 240, or 270.

In some embodiments, the peptide is provided to the retina from the vitreous for at least about (n) days after administration. In some embodiments, the peptide is provided to the retina from the vitreous for at least about (n) days after administration, wherein the peptide is detectable in the retina at day (n). In some embodiments, the peptide is provided to the retina from the vitreous for at least about (n) days after administration, wherein the peptide is detectable in the retina at day (n)±10 days. In some embodiments, it is not feasible to sample the vitreous humor of an individual receiving the peptide. In such instances, a comparison can therefore be made to a (n) value generated from a pig eye or a rabbit eye. Accordingly, in some embodiments, wherein the peptide is detectable in the retina corresponds to detection in a pig eye or rabbit eye at in the retina at day (n)±10 days, or at day (n). In some embodiments, the peptide is detectable by mass-spectrometry from a sample taken from the retina.

In some embodiments, (n) is about 14 to about 365. In some embodiments, (n) is about 30 to about 365. In some embodiments, (n) is at least 30. In some embodiments, (n) is at least 60. In some embodiments, (n) is at least 90. In some embodiments, (n) is at least 120. In some embodiments, (n) is at least 150. In some embodiments, (n) is at least 180. In some embodiments, (n) is at least 210. In some embodiments, (n) is at least 240. In some embodiments, (n) is at least 270. In some embodiments, (n) is at least 300. In some embodiments, (n) is at least 330. In some embodiments, (n) is at least 365. In some embodiments, (n) is greater than 30. In some embodiments, (n) is greater than 60. In some embodiments, (n) is greater than 90. In some embodiments, (n) is greater than 120. In some embodiments, (n) is greater than 150. In some embodiments, (n) is greater than 180. In some embodiments, (n) is greater than 210. In some embodiments, (n) is greater than 240. In some embodiments, (n) is greater than 270. In some embodiments, (n) is greater than 300. In some embodiments, (n) is greater than 330. In some embodiments, (n) is greater than 365. In some embodiments, (n) is 30 to 60, 30 to 90, 30 to 120, 30 to 150, 30 to 180, 30 to 210, 30 to 240, 30 to 270, 30 to 300, 30 to 330, 30 to 365, 60 to 90, 60 to 120, 60 to 150, 60 to 180, 60 to 210, 60 to 240, 60 to 270, 60 to 300, 60 to 330, 60 to 365, 90 to 120, 90 to 150, 90 to 180, 90 to 210, 90 to 240, 90 to 270, 90 to 300, 90 to 330, 90 to 365, 120 to 150, 120 to 180, 120 to 210, 120 to 240, 120 to 270, 120 to 300, 120 to 330, 120 to 365, 150 to 180, 150 to 210, 150 to 240, 150 to 270, 150 to 300, 150 to 330, 150 to 365, 180 to 210, 180 to 240, 180 to 270, 180 to 300, 180 to 330, 180 to 365, 210 to 240, 210 to 270, 210 to 300, 210 to 330, 210 to 365, 240 to 270, 240 to 300, 240 to 330, 240 to 365, 270 to 300, 270 to 330, 270 to 365, 300 to 330, 300 to 365, or 330 to 365. In some embodiments, (n) is 14, 30, 60, 90, 120, 150, 180, 210, 240, 270, 300, 330, or 365.

In some embodiments, the time between administrations can be a function of the half-life of the peptide in the vitreous humor. In some embodiments, (y) is 0.25, 0.5, 1, 2, 3, 4, 5, or 10 times the value of (z). In some embodiments, (y) is equal to the value of (z). In some embodiments, (y) is 2 times the value of (z). In some embodiments, (y) is 3 times the value of (z). In some embodiments, (y) is 4 times the value of (z). In some embodiments, (y) is 5 times the value of (z). In some embodiments, (y) is 10 times the value of (z).

In some embodiments, the time between doses can be a function of the presence of the peptide in the retinal tissue. In some embodiments, (y) is 0.25, 0.5, 1, 2, 3, 4, 5, or times the value of (n). In some embodiments, (y) is equal to the value of (n). In some embodiments, (y) is 2 times the value of (n). In some embodiments, (y) is 3 times the value of (n). In some embodiments, (y) is 4 times the value of (n). In some embodiments, (y) is 5 times the value of (n). In some embodiments, (y) is 10 times the value of (n). In some embodiments, a therapeutic effect is maintained over a period great than (n) days.

Provided here in are methods, comprising: administering a plurality of compositions to a vitreous humor of an eye, the plurality of compositions being administered no more frequently than (w) times in a year, and each of the plurality of compositions comprising a peptide comprising an amino acid sequence HHIYLGAVNYIY or variant sequence thereof, or a pharmaceutically-acceptable salt of the peptide. Also provided are methods, comprising: administering a plurality of compositions to a vitreous humor of an eye, the plurality of compositions being administered no more frequently than (w) times in a year, and each of the plurality of compositions comprising a peptide comprising having the structure of Formula I or a pharmaceutically-acceptable salt thereof. Further provided are methods, comprising: administering a plurality of compositions to a vitreous humor of an eye, the plurality of compositions being administered no more frequently than (w) times in a year, and each of the plurality of compositions comprising a peptide comprising having the structure of Formula III or a pharmaceutically-acceptable salt thereof. In some embodiments, the comprises a fixed number of administrations. In some embodiments, the comprises a fixed number of years.

In some embodiments, the method is a method of treating inflammation (e.g., Fas-mediated inflammation) in an eye (e.g., the retinal tissue of an eye). In some embodiments, the method is a method of inhibiting, reducing, and/or preventing inflammation (e.g., Fas-mediated inflammation) in an eye (e.g., the retinal tissue of an eye). In some embodiments, the method includes treating symptoms associated with inflammation in the eye (e.g., increased intraocular pressure, loss of and/or decrease in visual acuity, blurred vision, distorted vision, etc.). In some embodiments, retinal inflammation can be determined by observing the symptoms associated with inflammation in the eye (e.g., retinal degeneration, increased intraocular pressure, loss of and/or decrease in visual acuity, blurred vision, distorted vision, etc.) and/or by a biological assay detecting the presence of inflammatory molecules (e.g., inflammatory cytokines) in a sample (e.g., vitreous humor sample) taken from the eye.

In some embodiments, the method is a method of treating retinal cell loss (e.g., retinal pigment epithelial cells, retinal ganglion cells, and/or photoreceptors) in an eye (e.g., the retinal tissue of an eye). In some embodiments, the method is a method of inhibiting, reducing, and/or preventing retinal cell loss (e.g., retinal pigment epithelial cells, retinal ganglion cells, and/or photoreceptors) in an eye (e.g., the retinal tissue of an eye). In some embodiments, the method includes treating symptoms associated with retinal cell loss (e.g., retinal pigment epithelial cells, retinal ganglion cells, and/or photoreceptors) in the eye (e.g., retinal degeneration, increased intraocular pressure, loss of and/or decrease in visual acuity, blurred vision, distorted vision, etc.).

In some embodiments, the method is a method of treating retinal degeneration. In some embodiments, the method is a method of inhibiting, reducing, and/or preventing retinal degeneration. In some embodiments, the method includes treating symptoms associated with retinal degeneration (e.g., increased intraocular pressure, loss of and/or decrease in visual acuity, blurred vision, distorted vision, etc.). In some embodiments, retinal degeneration comprises the loss and/or death of retinal pigment epithelial cells, retinal ganglion cells, photoreceptors, or a combination thereof. In some embodiments, retinal degeneration comprises the loss and/or death of retinal pigment epithelial cells. In some embodiments, retinal degeneration comprises the loss and/or death of retinal ganglion cells. In some embodiments, retinal degeneration comprises the loss and/or death of photoreceptors. In some embodiments, retinal degeneration comprises the loss and/or death a combination of retinal pigment epithelial cells, retinal ganglion cells, and/or photoreceptors.

In some embodiments, the method is a method of treating an ocular disease, disorder, or condition. In some embodiments, the method is a method of inhibiting, reducing, and/or preventing an ocular disease, disorder, or condition. In some embodiments, the method includes treating symptoms associated with an ocular disease, disorder, or condition (e.g., retinal degeneration, increased intraocular pressure, loss of and/or decrease in visual acuity, blurred vision, distorted vision, etc.). In some embodiments, the ocular disease, disorder, or condition is a chronic ocular disease, disorder, or condition. In some embodiments, the ocular disease, disorder, or condition comprises the loss and/or death of retinal pigment epithelial cells, retinal ganglion cells, photoreceptors, or a combination thereof. In some embodiments, the ocular disease, disorder, or condition comprises the loss and/or death of retinal pigment epithelial cells. In some embodiments, the ocular disease, disorder, or condition comprises the loss and/or death of retinal ganglion cells. In some embodiments, the ocular disease, disorder, or condition comprises the loss and/or death of photoreceptors. In some embodiments, the ocular disease, disorder, or condition comprises the loss and/or death a combination of retinal pigment epithelial cells, retinal ganglion cells, and/or photoreceptors.

In some embodiments, the method is a method of treating glaucoma. In some embodiments, the method is a method of inhibiting, reducing, and/or preventing glaucoma. In some embodiments, the method includes treating symptoms associated with glaucoma (e.g., retinal degeneration, increased intraocular pressure, loss of and/or decrease in visual acuity, blurred vision, distorted vision, etc.). In some embodiments, the ocular disease, disorder, or condition comprises the loss and/or death of retinal ganglion cells.

In some embodiments, the method is a method of treating macular degeneration. In some embodiments, the method is a method of inhibiting, reducing, and/or preventing macular degeneration. In some embodiments, the method includes treating symptoms associated with macular degeneration (e.g., retinal degeneration, increased intraocular pressure, loss of and/or decrease in visual acuity, blurred vision, distorted vision, etc.). In some embodiments, the macular degeneration is age-related macular degeneration, non-exudative age-related macular degeneration, or exudative age-related macular degeneration. In some embodiments, the macular degeneration is age-related macular degeneration. In some embodiments, the macular degeneration is non-exudative age-related macular degeneration. In some embodiments, the macular degeneration is exudative age-related macular degeneration. In some embodiments, macular degeneration comprises the loss and/or death of retinal pigment epithelial cells, photoreceptors, or a combination thereof. In some embodiments, macular degeneration comprises the loss and/or death of retinal pigment epithelial cells. In some embodiments, macular degeneration comprises the loss and/or death of photoreceptors. In some embodiments, macular degeneration comprises the loss and/or death a combination of retinal pigment epithelial cells and photoreceptors.

In some embodiments, the method is a method of treating retinal detachment. In some embodiments, the method is a method of inhibiting, reducing, and/or preventing retinal detachment. In some embodiments, the method includes treating symptoms associated retinal detachment (e.g., retinal degeneration, increased intraocular pressure, loss of and/or decrease in visual acuity, blurred vision, distorted vision, etc.). In some embodiments, the retinal detachment is chronic retinal detachment. In some embodiments, retinal detachment comprises the loss and/or death of retinal pigment epithelial cells, retinal ganglion cells, photoreceptors, or a combination thereof. In some embodiments, retinal detachment comprises the loss and/or death of retinal pigment epithelial cells. In some embodiments, retinal detachment comprises the loss and/or death of retinal ganglion cells. In some embodiments, retinal detachment comprises the loss and/or death of photoreceptors. In some embodiments, retinal detachment comprises the loss and/or death a combination of retinal pigment epithelial cells, retinal ganglion cells, and/or photoreceptors.

In some embodiments, the method is a method of treating a loss in visual acuity. In some embodiments, the method is a method of treating a loss in visual acuity. In some embodiments, a loss in visual acuity is characterized by and/or equivalent to a reduction in visual acuity (e.g., a reduction from 20/100 to 20/200, or a reduction from 20/40 to 20/50, etc.). In some embodiments, the method is a method of treating ocular hypertension (e.g., elevated intraocular pressure). In some embodiments, the method is a method of treating ocular hypertension (e.g., elevated intraocular pressure). In some embodiments, ocular hypertension is characterized by an intraocular pressures of 21 mm Hg or greater. In some embodiments, ocular hypertension includes by an intraocular pressures of 18 mm Hg or greater.

In some embodiments, the method is a method of treating a loss in visual function. In some embodiments, the method is a method of treating a loss in visual function. In some embodiments, a loss in visual function is characterized by and/or equivalent to a reduction in visual function assessments (e.g., visual acuity, field of vision, contrast sensitivity, binocular function, and/or light/dark adaptation).

In some embodiments, (w) is 1 to 6. In some embodiments, (w) is 1 to 6. In some embodiments, (w) is at least 1. In some embodiments, (w) is at most 6. In some embodiments, (w) is 1 to 2, 1 to 3, 1 to 4, 1 to 5, 1 to 6, 2 to 3, 2 to 4, 2 to 5, 2 to 6, 3 to 4, 3 to 5, 3 to 6, 4 to 5, 4 to 6, or 5 to 6. In some embodiments, (w) is 1, 2, 3, 4, 5, or 6.

In some embodiments, the variant sequence comprises an amino acid substitution. In some embodiments, the variant sequence comprises one amino acid substitution. In some embodiments, the variant sequence comprises two amino acid substitutions. In some embodiments, the variant sequence comprises three amino acid substitutions.

In some embodiments, the peptide further comprises a modification. In some embodiments, comprises a modified amino acid or a non-natural amino acid. In some embodiments, the peptide comprises an amidated C-terminus. wherein the peptide has the structure of Formula I, or a pharmaceutically-acceptable salt thereof.

In some embodiments, the composition comprises the pharmaceutically-acceptable salt of the peptide. In some embodiments, the pharmaceutically-acceptable salt is an acetate salt. In some embodiments, the pharmaceutically-acceptable salt is a polyacetate salt. In some embodiments, the polyacetate salt is a triacetate salt. In some embodiments, the pharmaceutically-acceptable salt is a hydrochloride salt.

In some embodiments, the composition further comprises on or more excipients. In some embodiments, the composition further comprises a surfactant. In some embodiments, the surfactant is a non-ionic surfactant.

In some embodiments, the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer. In some embodiments, the surfactant is a polysorbate. In some embodiments, the surfactant is a polyethoxylated alcohol. In some embodiments, the surfactant is a polyoxyethylene-polyoxypropylene block copolymer. In some embodiments, the surfactant is an oxyethylated tertiary octylphenol formaldehyde polymer.

In some embodiments, the surfactant comprises Polysorbate 20, Poloxamer 407, Tyloxapol, or cremophor. In some embodiments, the surfactant comprises Polysorbate 20. In some embodiments, the surfactant comprises Poloxamer 407. In some embodiments, the surfactant comprises Tyloxapol. In some embodiments, the surfactant comprises cremophor. In some embodiments, the surfactant forms about 0.01% to about 20% weight/weight of the composition. In some embodiments, the surfactant forms about 0.05% to about 10% weight/weight of the composition. In some embodiments, the non-ionic surfactant is about 0.05% w/w of the composition to about 2% w/w of the composition. In some embodiments, the non-ionic surfactant is at least about 0.05% w/w of the composition. In some embodiments, the non-ionic surfactant is at most about 2% w/w of the composition. In some embodiments, the non-ionic surfactant is about 0.05% w/w of the composition to about 0.1% w/w of the composition, about 0.05% w/w of the composition to about 0.1% w/w of the composition, about 0.05% w/w of the composition to about 0.2% w/w of the composition, about 0.05% w/w of the composition to about 0.3% w/w of the composition, about 0.05% w/w of the composition to about 0.4% w/w of the composition, about 0.05% w/w of the composition to about 0.5% w/w of the composition, about 0.05% w/w of the composition to about 0.6% w/w of the composition, about 0.05% w/w of the composition to about 1% w/w of the composition, about 0.05% w/w of the composition to about 1.5% w/w of the composition, about 0.05% w/w of the composition to about 2% w/w of the composition, about 0.1% w/w of the composition to about 0.1% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.1% w/w of the composition to about 0.2% w/w of the composition, about 0.1% w/w of the composition to about 0.3% w/w of the composition, about 0.1% w/w of the composition to about 0.4% w/w of the composition, about 0.1% w/w of the composition to about 0.5% w/w of the composition, about 0.1% w/w of the composition to about 0.6% w/w of the composition, about 0.1% w/w of the composition to about 1% w/w of the composition, about 0.1% w/w of the composition to about 1.5% w/w of the composition, about 0.1% w/w of the composition to about 2% w/w of the composition, about 0.2% w/w of the composition to about 0.3% w/w of the composition, about 0.2% w/w of the composition to about 0.4% w/w of the composition, about 0.2% w/w of the composition to about 0.5% w/w of the composition, about 0.2% w/w of the composition to about 0.6% w/w of the composition, about 0.2% w/w of the composition to about 1% w/w of the composition, about 0.2% w/w of the composition to about 1.5% w/w of the composition, about 0.2% w/w of the composition to about 2% w/w of the composition, about 0.3% w/w of the composition to about 0.4% w/w of the composition, about 0.3% w/w of the composition to about 0.5% w/w of the composition, about 0.3% w/w of the composition to about 0.6% w/w of the composition, about 0.3% w/w of the composition to about 1% w/w of the composition, about 0.3% w/w of the composition to about 1.5% w/w of the composition, about 0.3% w/w of the composition to about 2% w/w of the composition, about 0.4% w/w of the composition to about 0.5% w/w of the composition, about 0.4% w/w of the composition to about 0.6% w/w of the composition, about 0.4% w/w of the composition to about 1% w/w of the composition, about 0.4% w/w of the composition to about 1.5% w/w of the composition, about 0.4% w/w of the composition to about 2% w/w of the composition, about 0.5% w/w of the composition to about 0.6% w/w of the composition, about 0.5% w/w of the composition to about 1% w/w of the composition, about 0.5% w/w of the composition to about 1.5% w/w of the composition, about 0.5% w/w of the composition to about 2% w/w of the composition, about 0.6% w/w of the composition to about 1% w/w of the composition, about 0.6% w/w of the composition to about 1.5% w/w of the composition, about 0.6% w/w of the composition to about 2% w/w of the composition, about 1% w/w of the composition to about 1.5% w/w of the composition, about 1% w/w of the composition to about 2% w/w of the composition, or about 1.5% w/w of the composition to about 2% w/w of the composition. In some embodiments, the non-ionic surfactant is about 0.05% w/w of the composition, about 0.1% w/w of the composition, about 0.1% w/w of the composition, about 0.2% w/w of the composition, about 0.3% w/w of the composition, about 0.4% w/w of the composition, about 0.5% w/w of the composition, about 0.6% w/w of the composition, about 1% w/w of the composition, about 1.5% w/w of the composition, or about 2% w/w of the composition.

In some embodiments, the composition further comprises a tonicity adjusting agent, a buffering agent, or a combination thereof (e.g., as described herein, including any of the described ranges and/or values within the described ranges). In some embodiments, the composition is buffered at a pH of 2.5 to 7.5.

In some embodiments, the composition comprises 5 micrograms (ug) to 10,000 ug of the peptide. In some embodiments, the composition comprises about 5 ug of the peptide to about 300 ug of the peptide. In some embodiments, the composition comprises at least about 5 ug of the peptide. In some embodiments, the composition comprises at least about 25 ug of the peptide. In some embodiments, the composition comprises at least about 50 ug of the peptide. In some embodiments, the composition comprises at least about 100 ug of the peptide. In some embodiments, the composition comprises at least about 125 ug of the peptide. In some embodiments, the composition comprises at least about 150 ug of the peptide. In some embodiments, the composition comprises at least about 200 ug of the peptide. In some embodiments, the composition comprises at least about 250 ug of the peptide. In some embodiments, the composition comprises at least about 300 ug of the peptide.

In some embodiments, the composition comprises about 5 ug of the peptide to about 10 ug of the peptide, about 5 ug of the peptide to about 25 ug of the peptide, about 5 ug of the peptide to about 50 ug of the peptide, about 5 ug of the peptide to about 75 ug of the peptide, about 5 ug of the peptide to about 100 ug of the peptide, about 5 ug of the peptide to about 150 ug of the peptide, about 5 ug of the peptide to about 200 ug of the peptide, about 5 ug of the peptide to about 250 ug of the peptide, about 5 ug of the peptide to about 300 ug of the peptide, about 10 ug of the peptide to about 25 ug of the peptide, about 10 ug of the peptide to about 50 ug of the peptide, about 10 ug of the peptide to about 75 ug of the peptide, about 10 ug of the peptide to about 100 ug of the peptide, about 10 ug of the peptide to about 150 ug of the peptide, about 10 ug of the peptide to about 200 ug of the peptide, about 10 ug of the peptide to about 250 ug of the peptide, about 10 ug of the peptide to about 300 ug of the peptide, about 25 ug of the peptide to about 50 ug of the peptide, about 25 ug of the peptide to about 75 ug of the peptide, about 25 ug of the peptide to about 100 ug of the peptide, about 25 ug of the peptide to about 150 ug of the peptide, about 25 ug of the peptide to about 200 ug of the peptide, about 25 ug of the peptide to about 250 ug of the peptide, about 25 ug of the peptide to about 300 ug of the peptide, about 50 ug of the peptide to about 75 ug of the peptide, about 50 ug of the peptide to about 100 ug of the peptide, about 50 ug of the peptide to about 150 ug of the peptide, about 50 ug of the peptide to about 200 ug of the peptide, about 50 ug of the peptide to about 250 ug of the peptide, about 50 ug of the peptide to about 300 ug of the peptide, about 75 ug of the peptide to about 100 ug of the peptide, about 75 ug of the peptide to about 150 ug of the peptide, about 75 ug of the peptide to about 200 ug of the peptide, about 75 ug of the peptide to about 250 ug of the peptide, about 75 ug of the peptide to about 300 ug of the peptide, about 100 ug of the peptide to about 150 ug of the peptide, about 100 ug of the peptide to about 200 ug of the peptide, about 100 ug of the peptide to about 250 ug of the peptide, about 100 ug of the peptide to about 300 ug of the peptide, about 150 ug of the peptide to about 200 ug of the peptide, about 150 ug of the peptide to about 250 ug of the peptide, about 150 ug of the peptide to about 300 ug of the peptide, about 200 ug of the peptide to about 250 ug of the peptide, about 200 ug of the peptide to about 300 ug of the peptide, or about 250 ug of the peptide to about 300 ug of the peptide. In some embodiments, the composition comprises about 5 ug of the peptide, about 10 ug of the peptide, about 25 ug of the peptide, about 50 ug of the peptide, about 75 ug of the peptide, about 100 ug of the peptide, about 150 ug of the peptide, about 200 ug of the peptide, about 250 ug of the peptide, or about 300 ug of the peptide.

In some embodiments, the peptide is present at a concentration 0.1 milligrams per milliliter (mg/mL) to 5.0 mg/mL.

As used herein, individual is synonymous with patient and/or subject and includes and/or refers to a human and may be a human that has been diagnosed as needing to treat a disease or condition as disclosed herein. However, examples are not limited to humans and include, chimpanzees, marmosets, cows, horses, sheep, goats, pigs, rabbits, dogs, cats, rats, mice, guinea pigs, and the like. The individual is typically a human and may be a human that has been diagnosed as needing to treat a disease or condition as disclosed herein.

As used herein, “a” or “an” when used in conjunction with the term “comprising” in the claims and/or the specification includes and/or refers to “one” and also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used herein, the term “about” in the context of a given value or range includes and/or refers to a value or range that is within 20%, preferably within 10%, and more preferably within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

As used herein, a “sample” includes and/or refers to any fluid or liquid sample which is being analyzed in order to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

### EXAMPLES

### Example I—Pharmacokinetics Studies in Rabbit Eye

### Objective

A study was performed to evaluate the ocular tissue concentration of a peptide having an acetate salt of the structure of Formula III (ONL1204) up to 168 days following a single intravitreal injection to both eyes and a repeat intravitreal injection on Day 90 in a select group. A total of 33 rabbits were evaluated on study to evaluate ONL1204 over 168 days.

### Study Design

Animals were observed for any abnormal observations prior to dosing, approximately weekly (for surviving animals through Week 12), and at euthanasia. Body weights were measured and recorded prior to first dose, approximately weekly (for surviving animals through Week 12), and prior to euthanasia. No ONL1204-related effects on body weights or clinical observations were evident during the study. Ophthalmic examinations were performed using the Hackett-McDonald Scoring System on Days 0, 7, 14, 28, 90, 97, 151, and 168. Pharmacokinetic analyses were performed on the vitreous humor concentration data.

Animals received a single dose of the test article, ONL1204, at 2 mg/mL (0.1 mg/eye once on Day 1 via intravitreal (IVT) injection). Seven animals received a repeat IVT dose at 2 mg/mL (0.1 mg/eye) on Day 90. The formulation contained the 2 mg/mL ONL1204 in Vehicle (4.5% mannitol/0.4% poloxamer-407, 10 mM acetic acid pH 4.5).

Vitreous humor, retina, and choroid were collected on days 1, 7, 14, 28, 42, 61, 90, 97, 151, and 168. Eyes were enucleated, snap frozen using liquid nitrogen, and kept on dry ice or frozen at −80° C. until dissected for collection of aqueous humor, vitreous humor, retina, choroid, iris-ciliary body (ICB), and lens.

### Sample Preparation

To homogenize ocular tissue samples, weighed amounts of control bovine vitreous humor, retina, and choroid were homogenized in USA scientific impact resistant microtubes containing 2.8 mm ceramic beads. Unknown rabbit vitreous humor, retina, and choroid samples were homogenized in USA scientific impact resistant microtubes containing 2.8 mm ceramic beads. Each vitreous humor sample was homogenized after the addition of 5000 microliters (uL) diluent (acetonitrile:water:1 M hydrochloric acid (70:20:10, v/v/v). Retina tissues were homogenized after the addition of diluent (IPA:NH4OH (1000:1, v/v)) at a 1:5 ratio (parts tissue to parts diluent), and choroid samples were homogenized following addition of diluent at a 1:19 ratio. Tissues were homogenized at 5500 rpm for 3×30 second cycles with 20 second pauses between cycles until homogenized. Choroid went through two runs of homogenization, and retina and vitreous humor samples went through one run of homogenization.

### Preparation of Calibration Stock and Working Standards:

A stock calibration standard was prepared in dimethylsulfoxide (DMSO) at a concentration of 500 ug/mL for ONL1204. Working calibration standards were prepared for vitreous humor by serial dilution of working stock solution with acetonitrile:water:formic acid (25:75:0.1, v/v/v) over a range of 500 ng/ml to 500,000 ng/mL ONL1204. Working calibration standards were prepared for choroid and retina by serial dilution of working stock solution with acetonitrile:water:formic acid (25:75:0.1, v/v/v) over a range of 5.00 ng/mL to 1,000 ng/mL ONL1204.
   Preparation of Standards, Unknowns, Blanks, and Blanks with Internal Standard for Vitreous Humor Analysis:
In a polypropylene tube, 10 uL of working calibration standard was added to 90 uL control blank vitreous humor. For blanks and blanks with internal standard, 100 uL of control blank bovine vitreous humor was added. Five hundred (500) uL of acetonitrile:water:1 M hydrochloric acid (70:20:10, v/v/v) was added to each standard and 500 uL of acetonitrile:water: 1 M hydrochloric acid (70:20:10, v/v/v) was added to blanks. Standards and blanks were vortex mixed. One hundred (100) uL of each standard, blank or unknown vitreous humor sample homogenate was then aliquoted. One hundred (100) uL of IPA:NH₄OH (1000:1, v/v) was added to each sample. The samples were vortex mixed for 1 minute, then centrifuged for 10 minutes at 4,000 rpm (4° C.). In an autosampler plate, 50.0 uL of working internal standard (WIS) (5,000 ng/mL APi1887 in acetonitrile:water:formic acid (25:75:0.1, v/v/v)) was added to 50.0 uL supernatant. Fifty (50.0) uL of acetonitrile:water:1 M hydrochloric acid (70:20:10, v/v/v) was added to blank without IS. Two hundred (200) uL of water was added. The samples were then mixed using a multichannel pipette, and supernatant transferred to an autosampler plate for analysis.
   Preparation of Standards, Unknowns, Blanks, and Blanks with Internal Standard for Retina and Choroid:

In a 96-well plate, 100 uL of control retina or choroid homogenate was added for calibration samples and unknowns. For blanks and blanks with internal standard, 100 uL of control bovine retina or choroid tissue homogenate was added. Twenty (20) uL of WIS (5,000 ng/ml APi1887 in acetonitrile:water:formic acid [25:75:0.1, v/v/v]) was added to each calibration standard and blank with IS sample. Twenty (20) uL acetonitrile:water:formic acid (25:75:0.1, v/v/v) was added to each blank without IS sample. Three-hundred (300) uL of IPA:NH₄OH (1000:1, v/v) was added to each choroid or retina sample. The samples were then vortex mixed for 5 minutes. The samples were allowed to set for 15 minutes, vortex mixed for an additional minute, and centrifuged for 10 minutes at 4,000 rpm (4° C.).

Three-hundred-fifty (350) uL of each sample was then aliquoted to a 96-well plate. The samples were dried with nitrogen at 35° C. Two-hundred (200) uL of acetonitrile:water:formic acid (25:75:0.1, v/v/v) was added to reconstitute each sample. The samples were then vortex mixed for 4 minutes, and transferred to a 96-well autosampler plate for analysis.

### MS Conditions:

Instrument Applied Biosystems API 4000 LC-MS/MS
Ionization Turbo Spray (ESI)
MS Mode Multiple Reaction Monitoring (MRM)
Polarity—Positive
Scan Time—100 msec/transition
Transition—732.0⇒585.3 for ONL-1204 CE 30, 739.9⇒593.5 for APi1887 (IS) CE 31

### Ionization Settings:

Collision Gas—8 (arbitrary units)
Curtain Gas—12 (arbitrary units)
Gas 1—50 (arbitrary units)
Gas 2—50 (arbitrary units)
Spray Voltage—5000 V
Source Temperature—500° C.
Declustering Potential (DP)—80 V
Entrance Potential—8V
Collision Exit Potential—15

### HPLC Conditions:

Instrument—Agilent 1100 Series HPLC System
Injection Volume—10 μL
Mobile phase A: Water:Formic acid (1000:1, v/v), B: Acetonitrile: IPA: Formic acid
(90:10:0.1, v/v/v)
Gradient-0.00-0.50 minutes: isocratic 10% B, 0.50-0.60 minutes: gradient to 50% B, 0.60-3.00 minutes: isocratic 50% B, 3.00-3.10 minutes: gradient to 90% B, 3.10-4.00 minutes: washout 90% B, 4.00-4.10 minutes: re-equilibrate to 10% B, 4.10-7.50
minutes: isocratic 10% B
Flow rate—0.400 mL/minute
Column-Waters Atlantis t3 C18, 3.5 μm, 100×2.1 mm
Needle Wash-Acetonitrile: Formic acid (1000:1, v/v)

### Pharmacokinetics

Based on the results, the t1/2 for animals receiving a single dose was 279.8 days (0.1 mg/eye, days 0 to 168). Table 1 shows drug concentrations in the vitreous humor and retinal issue. ONL1204 was detected in the vitreous humor across all time points analyzed. Retinal tissue is generally difficult to assay, wherein detection in the retina represents the minimal amount of ONL1204 present (i.e., the values do not likely overestimate the concentration of ONL1204 in retinal tissue (limit of quantification (LLOQ)=0.500 ng/ml, 3.00 ng/g for the retina, unless marked with “<LLOQ*” which indicates LLOQ=1.00 ng/ml, 6.0 ng/g; ** denotes animal group receiving a second injection at day 90). Nonetheless, ONL1204 was detected in retinal tissue as well as the vitreous humor after 168 days.

TABLE 1

Conc. of ONL 1204

| | | Vitreous | |
|---|---|---|---|
| | | Humor | Retina |
| Sample ID** | Day | (ug) | (ng/g) |
| 1A-Right | 1 | 76 | <LLOQ |
| 1A-Left | 1 | 48.3 | 12.7 |
| 1B-Right | 1 | 80.9 | <LLOQ |
| 1B-Left | 1 | 89.6 | <LLOQ |
| 1C-Right | 1 | 74.9 | <LLOQ |
| 1C-Left | 1 | 77.1 | <LLOQ |
| 2A-Right | 7 | 83 | <LLOQ |
| 2A-Left | 7 | 41.6 | <LLOQ |
| 2B-Right | 7 | 82.6 | <LLOQ |
| 2B-Left | 7 | 83.6 | <LLOQ |
| 2C-Right | 7 | 79.2 | <LLOQ |
| 2C-Left | 7 | 67.8 | 85.2 |
| 3A-Right | 14 | 67.3 | <LLOQ |
| 3A-Left | 14 | 67.4 | <LLOQ |
| 3B-Right | 14 | 54.2 | <LLOQ |
| 3B-Left | 14 | 70.9 | <LLOQ |
| 3C-Right | 14 | 63.2 | 5.95 |
| 3C-Left | 14 | 74.5 | <LLOQ |
| 4A-Right | 28 | 76.9 | <LLOQ |
| 4A-Left | 28 | 70.2 | <LLOQ |
| 4B-Right | 28 | 74.4 | <LLOQ |
| 4B-Left | 28 | 82 | <LLOQ |
| 4C-Right | 28 | 69.9 | <LLOQ |
| 4C-Left | 28 | 63.9 | <LLOQ |
| 5A-Right | 42 | 71.7 | <LLOQ |
| 5A-Left | 42 | 69.3 | <LLOQ |
| 5B-Right | 42 | 65.3 | <LLOQ |
| 5B-Left | 42 | 75.7 | <LLOQ |
| 5C-Right | 42 | 60.9 | <LLOQ |
| 5C-Left | 42 | 62.3 | <LLOQ |
| 6A-Right | 61 | 54.8 | <LLOQ |
| 6A-Left | 61 | 55.2 | <LLOQ |
| 6B-Right | 61 | 58.3 | <LLOQ |
| 6B-Left | 61 | 54.5 | <LLOQ |
| 6C-Right | 61 | 59.9 | <LLOQ |
| 7A-Right | 90 | 63.7 | <LLOQ* |
| 7A-Left | 90 | 61.5 | <LLOQ* |
| 8A-Right | 90 | 54.1 | <LLOQ* |
| 8A-Left | 90 | 51 | <LLOQ* |
| 7B-Right** | 97 | 131 | 9.55 |
| 7B-Left** | 97 | 135 | <LLOQ* |
| 8B-Right** | 97 | 127 | 8.52 |
| 8B-Left** | 97 | 113 | <LLOQ* |
| 8C-Right** | 151 | 99.1 | <LLOQ* |
| 8C-Left** | 151 | 103 | 15.6 |
| 9A-Right** | 151 | 113 | 52.9 |
| 9A-Left** | 151 | 124 | 9.54 |
| 9B-Right | 168 | 99.8 | 83.9 |
| 9B-Left | 168 | 103 | <LLOQ* |
| 9C-Right | 168 | 75.9 | <LLOQ* |
| 9C-Left | 168 | 85.6 | <LLOQ* |
| 10B-Right | 168 | 48.7 | 32.9 |
| 10B-Left | 168 | 45.7 | 8.4 |
| 10C-Right | 168 | 55.8 | <LLOQ* |
| 10C-Left | 168 | 44.7 | <LLOQ* |

Right or left designates the right or left eye, respectively.

### Example 2—Sodium Iodate (NaIO₃) Challenge Study

### Objective

The objective of this study was to test the duration of activity (e.g., protection) of ONL1204 in the male rabbit NaIO₃ model by treating animals with test article (ONL1204) on Day 0 and staggering the day of NaIO₃ insult to assess duration of protection offered by ONL1204. Demonstrating a protective effect of ONL1204 at extended timepoints would support the use of ONL1204 ophthalmic solution in chronic ophthalmic diseases.

### Study Design

### Test Article:

Test Article: ONL1204 Formulation
Administration Route: 50 ul by IVT injection
Frequency of Administration: Single dose on Day 0
Formulations: Group 1-4; 9: 2 mg/ml ONL1204 in 4.5% mannitol, 0.4% poloxamer 407, 10 mM acetate buffer (buffer?) pH 4.5, Group 5-8: 0.5 mg/ml ONL1204 in 4.5% mannitol, 0.4% poloxamer 407, 10 mM acetate pH 4.5
Dose: Group 1-4; 9: 100 ug per eye, Group 5-8: 25 ug per eye

### NaIO₃ Solution:

Challenge Article: NaIO₃
Administration Route: IV
Frequency of Administration: Day 4 (day 7 endpoint), 14 (day 17 endpoint), 28 (day 31 endpoint), 59 (day 61 endpoint), or 78 (day 81 endpoint (rechallenged group)).
Solution: 20 mg/ml in 0.9% Injectable Saline
Dose: 17 or 18 mg/kg body weight

### Test System:

Species: Rabbit
Expected Age: Animals were between 3 and 4 months of age
Expected Body Range: Approximately 2.0 Kg, Identification Animals were individually housed and identified via cage card and ear ID and/or ear tattoo.
Experimental History: Purpose-bred and experimentally naïve at the onset of the study

### Intravitreal Injection:

On Day 0, eyes were anesthetized with proparacaine and pupils dilated with tropicamide. Animals were anesthetized with isoflurane vapors to effect. A few drops of Betadine were placed on the eye and then rinsed with ophthalmic eye wash. A clean speculum was placed in the eye (rinsed in Betadine and sterile saline) and the temporal sclera was marked with calipers 3.5 mm from the corneal border. A BD Ultra-Fine 31 g, 3/10 cc, 5/16 in, Insulin Syringe with 50 ul of test article of 0.5 mg/ml or 2.0 mg/ml ONL1204 was inserted though indentation into the mid-vitreous approximately 62° from the horizontal axis (28° from the vertical axis) and test article was injected into the vitreous. In order to reduce the potential for reflux of the test article the needle remained in the vitreous for at least 10 seconds following injection. No reflux was noted on the dosing record.

### Model Induction:

A 20 mg/ml solution of NaIO₃ was prepared in 0.9% injectable saline aliquoted into 4 batches and frozen at −20° C. For Groups 1-8, 1 batch of NaIO₃ was thawed prior to injection. Groups 9-10 were added after the Study was underway therefore a new solution of 20 mg/ml NaIO₃ was prepared in 0.9% injectable saline and frozen at −20° C. and thawed prior to injection. Test article and NaIO₃ were injected into the left ear.

Animals were weighed and anesthetized with isoflurane vapors to effect. The left ear was shaved and swabbed with 10% alcohol prior to injection. An indwelling catheter was placed in the left marginal ear vein and flushed with 0.9% injectable saline. Animals were weighed just prior to dosing and Groups 1-8 received 18 mg NaIO₃/kg bodyweight followed by a flush of 0.3 ml 0.9% injectable saline. Groups 9-10 received 16 mg NaIO₃/kg bodyweight followed by a flush of 0.3 ml 0.9% injectable saline. All injections were performed without complication and no reflux or leakage was noted.

### Fluorescein Angiography:

Eyes were anesthetized with proparacaine and pupils dilated with tropicamide and phenylephrine. Animals were anesthetized with isoflurane vapors to effect. 50 ul of 25% sodium fluorescein was injected into the marginal ear vein and allowed to circulate for at least 2 minutes prior to imaging. In animals with intact retinal pigment epithelial cell pigmentation, it is not possible to stimulate or visualize the light emitted by fluorescein circulating in the choroidal vasculature. NaIO₃ causes one or more regions of retinal pigment epithelial cell death and depigmentation which creates a window into the choroid. Images were captured to best map the full extent of visible choroid/retinal pigment epithelial cell death in both eyes.

### Data Analysis:

The area of RPE depigmentation is reported as a unitless value provided by the ImageJ software. In certain cases where the damage extended well into the periphery it was not possible to image the entire edge of the affected area. In some cases, a typical lesion was not created in either eye. In these cases, it is possible to measure the accumulation of lipofuscin, the phagocytized shed outer segments normally processed by the RPE, which builds up in unhealthy RPE. In these cases, ImageJ was used to highlight the autofluorescence around the Optic Nerve Head by thresholding the image with a 100/20 window level. The resulting area was then measured.

### Results

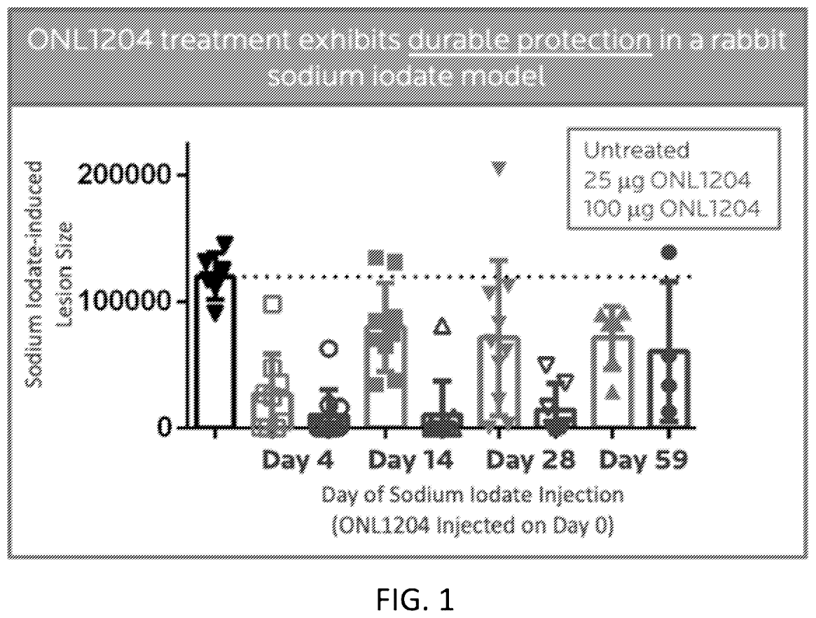

The mean lesion size in both eyes of 3 male rabbits, N=6, were used as negative control, against which the protective effect of ONL1204 treatment was compared. On Day 4, there were equally small lesions in both treatment groups and no statistical difference in lesion area. Both Groups were significantly lower than negative control. On Day 14, animals treated with 100 ug had significantly smaller lesion size then both negative controls and those treated with 25 ug. On Day 28, animals treated with 100 ug had significantly smaller lesion size then both negative control animals and those treated with 25 ug. A substantive reduction in mean lesion size was observed across challenge time points. On Day 59, the mean lesion size for animals treated with 25 ug and 100 ug was 40% and 50% smaller respectively compared to negative controls. Results are shown in FIG. 1.

### Example 3—Pharmacokinetics Studies in Minipig

### Objective

A study was performed in minipig to evaluate the ocular tissue concentrations of a peptide having the structure of Formula III (ONL1204) following a single intravitreal injection.

### Study Design

Animals received a single dose of the test article, ONL1204, at 2 mg/mL (0.1 mg/eye once on Day 1 via intravitreal (IVT) injection). Seven animals received a repeat IVT dose at 2 mg/mL (0.1 mg/eye) on Day 90. The formulation contained the 2 mg/mL ONL1204 in Vehicle (4.5% mannitol/0.4% poloxamer-407, 10 mM acetic acid pH 4.5). Vitreous humor, retina, and choroid were collected on days 1, 7, 14, 28, and 88.

### Pharmacokinetics

Table 2 shows drug concentration results in the vitreous humor, retinal issue, and choroid. ONL1204 was detected in the vitreous humor across all time points analyzed. Due to assay limitations, detection in the retina represents the minimal amount of ONL1204 present (i.e., the values do not likely overestimate the concentration of ONL1204 in retinal tissue) (limit of quantification (LLOQ) for vitreous humor=0.5 ug/mL; LLOQ for retina=0.500 ng/ml, 2.5 ng/g unless noted by “<LLOQ*” indicating LLOQ=1.00 ng/ml, 5.0 ng/g; LLOQ for choroid=1.00 ng/ml, 20.0 ng/g).

TABLE 2

| | | | Concentration | | |
|---|---|---|---|---|---|
| | | | Vitreous | | |
| | Dose | | humor | Retina | Choroid |
| Animal ID | (ug/eye) | Day | (μg) | (ng/g) | (ng/g) |
| 1 | Vehicle | 6 | <LLOQ | <LLOQ | <LLOQ |
| 2 | | 6 | <LLOQ | <LLOQ | <LLOQ |
| 3 | | 6 | <LLOQ | <LLOQ | <LLOQ |
| 4 | | 28 | <LLOQ | <LLOQ | <LLOQ |
| 5 | | 28 | <LLOQ | <LLOQ | <LLOQ |
| 6 | | 28 | <LLOQ | <LLOQ | <LLOQ |
| 7 | | 88 | <LLOQ | <LLOQ* | <LLOQ |
| 8 | | 88 | <LLOQ | <LLOQ* | <LLOQ |
| 9 | | 88 | <LLOQ | <LLOQ* | <LLOQ |
| 10 | 200 | 6 | 130 | <LLOQ | 32.4 |
| 11 | | 6 | 106 | 4.16 | 352 |
| 12 | | 6 | 111 | 49.3 | 182 |
| 13 | | 28 | 156 | 6.14 | 166 |
| 14 | | 28 | 133 | <LLOQ | <LLOQ |
| 15 | | 28 | 107 | <LLOQ | 33.5 |
| 16 | | 88 | 95.1 | <LLOQ* | 31 |
| 17 | | 88 | 75.5 | <LLOQ* | 62.5 |
| 18 | | 88 | 54.0 | <LLOQ* | 21 |
| 19 | 300 | 6 | 205 | 9.5 | <LLOQ |
| 20 | | 6 | 141 | 12.1 | 125 |
| 21 | | 6 | 153 | <LLOQ | 178 |
| 22 | | 28 | 124 | 39.3 | 77.7 |
| 23 | | 28 | 232 | 25.5 | 119 |
| 24 | | 28 | 184 | 6.59 | 50.5 |
| 25 | | 88 | 88.3 | <LLOQ* | <LLOQ |
| 26 | | 88 | 137 | <LLOQ* | 89.4 |
| 27 | | 88 | 99.6 | <LLOQ* | <LLOQ |

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

### Sequences

| SEQ ID NO | SEQUENCE |
|---|---|
| 1 | HHIYLGAVNYIY |
| 2 | YIYNVAGLYIHH |
| 3 | HHIYLGAVNYIY |
| 4 | YIYNVAGLYIHH |
| 5 | YIYNVAGLYIHH |
| 6 | YVYNVAGLYVHH |
| 7 | YVYNVAGLYVHH |
| 8 | YIYNVAGLYIHH |
| 9 | YVYNVAGLYVHH |

1. A method of treating an ocular disease, disorder, or condition, comprising:
(a) administering a first composition comprising a peptide comprising an amino acid sequence HHIYLGAVNYIY or a variant sequence thereof, or a pharmaceutically-acceptable salt of the peptide, to a vitreous humor of an eye; and
(b) administering at least a second composition comprising the peptide to the vitreous humor of the eye, wherein the at least second dose is administered no less than about 30 to 365 days after the first administration.

2. The method of claim 1, wherein the at least second dose is administered no less than about 90 to 180 days after the first administration

3. A method of treating an ocular disease, disorder, or condition, comprising:
administering a plurality of compositions to a vitreous humor of an eye, the plurality of compositions being administered no more frequently than once every about 30 to 365 days, and each of the plurality of compositions comprising a peptide comprising an amino acid sequence HHIYLGAVNYIY or variant sequence thereof, or a pharmaceutically-acceptable salt of the peptide.

4. The method of claim 3, the plurality of compositions being administered no more frequently than once every about 90 to 180 days.

5. The method of any one of claims 1 to 4, wherein the peptide has a half-life in the vitreous humor of (e.g., at least) about 30 to 300 days.

6. The method of claim 5, the peptide has a half-life in the vitreous humor of (e.g., at least) about 90 to 300 days.

7. The method of any one of claims 1 to 6, wherein the method comprises using the vitreous humor as a depot to provide the peptide to retinal tissue in the eye, wherein the peptide is present in the vitreous humor for about 30 to 365 days after administration.

8. The method of any one of claims 1 to 7, wherein the peptide is provided to the retina from the vitreous humor for at least about 30 to 365 days after administration.

9. The method of any one of claims 1 to 8, wherein the peptide is present in the vitreous humor at about 90 to 365 days after administration.

10. A method of treating an ocular disease, disorder, or condition, comprising:
administering a composition comprising a peptide comprising an amino acid sequence HHIYLGAVNYIY or a variant sequence thereof, or a pharmaceutically-acceptable salt of the peptide, to a vitreous humor of an eye, thereby providing the peptide to retinal tissue in the eye, wherein the peptide has a half-life in the vitreous humor of about 30 to 300 days.

11. The method of claim 10, wherein the peptide has a half-life in the vitreous humor of at least about 90 to 300 days.

12. The method of claim 10 or 11, wherein the method comprises using the vitreous humor as a depot to provide a therapeutically-effective amount of the peptide to the retinal tissue.

13. A method of treating an ocular disease, disorder, or condition or a symptom thereof, comprising administering a composition comprising a peptide comprising an amino acid sequence HHIYLGAVNYIY or a variant sequence thereof, or a pharmaceutically-acceptable salt of the peptide, to a vitreous humor of an eye, wherein the composition is administered no greater than 4 times a year.

14. The method of any one of claims 11 to 13, wherein the peptide is provided to the retina from the vitreous humor for at least about 14 to 365 days after administration.

15. The method of claim 14, wherein the peptide is provided to the retina from the vitreous humor for at least about 30 days after administration.

16. The method of claim 14, wherein the peptide is provided to the retina from the vitreous humor for at least about 90 days after administration.

17. The method of any one of claims 1 to 16, wherein The method of any one of claims 1 to 14, wherein the ocular disease, disorder, or condition comprises inflammation in retinal tissue and/or a symptom thereof.

18. The method of any one of claims 1 to 17, wherein the ocular disease, disorder, or condition comprises photoreceptor cell death and/or a symptom thereof.

19. The method of any one of claims 1 to 18, wherein the ocular disease, disorder, or condition comprises retinal degeneration and/or a symptom thereof.

20. The method of any one of claims 1 to 19, wherein the ocular disease, disorder, or condition comprises a loss and/or decrease in visual acuity.

21. The method of any one of claims 1 to 20, wherein the ocular disease, disorder, or condition comprises macular degeneration and/or a symptom thereof.

22. The method of any one of claims 1 to 21, wherein the ocular disease, disorder, or condition comprises glaucoma and/or a symptom thereof.

23. The method of any one of claims 1 to 22, wherein the variant sequence comprises an amino acid substitution.

24. The method of claim 23, wherein the variant sequence comprises one amino acid substitution.

25. The method of claim 23, wherein the variant sequence comprises two amino acid substitutions.

26. The method of claim 23, wherein the variant sequence comprises three amino acid substitutions.

27. The method of any one of claims 1 to 26, wherein the peptide further comprises a modification.

28. The method of claim 27, wherein the modification comprises a modified amino acid.

29. The method of any one of claims 1 to 28, wherein the peptide comprises an amidated C-terminus.

30. The method of any one of claims 1 to 29, wherein the peptide has the structure of Formula I or a pharmaceutically-acceptable salt thereof.

31. The method of any one of claims 1 to 30, wherein the peptide has the structure of Formula III: 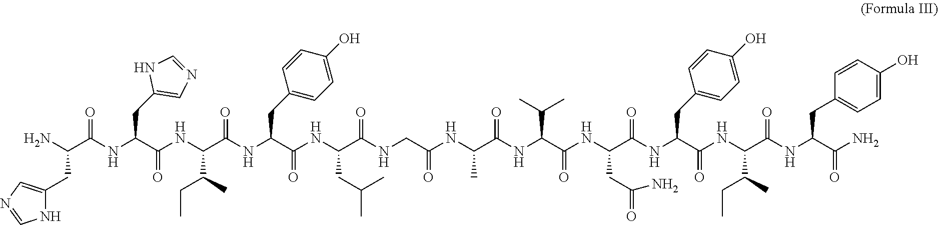 [SMILES: CC[C@H](C)[C@H](NC(=O)[C@H](Cc1ccc(O)cc1)NC(=O)[C@H](CC(N)=O)NC(=O)[C@@H](NC(=O)[C@H](C)NC(=O)CNC(=O)[C@H](CC(C)C)NC(=O)[C@H](Cc1ccc(O)cc1)NC(=O)[C@@H](NC(=O)[C@H](Cc1cnc[nH]1)NC(=O)[C@@H](N)Cc1cnc[nH]1)[C@@H](C)CC)C(C)C)C(=O)N[C@@H](Cc1ccc(O)cc1)C(N)=O]
or a pharmaceutically-acceptable salt thereof.

32. The method of any one of claims 1 to 31, wherein the composition comprises the pharmaceutically-acceptable salt of the peptide.

33. The method of claim 32, wherein the pharmaceutically-acceptable salt is an acetate salt.

34. The method of claim 33, wherein the pharmaceutically-acceptable salt is a polyacetate salt.

35. The method of claim 33, wherein the polyacetate salt is a triacetate salt.

36. The method of claim 32, wherein the pharmaceutically-acceptable salt is a hydrochloride salt.

37. The method of any one of claims 1 to 36, wherein the composition further comprises one or more excipients.

38. The method of any one of claims 1 to 37, wherein the composition further comprises a surfactant.

39. The method of claim 38, wherein the surfactant is a non-ionic surfactant.

40. The method of claim 38, wherein the surfactant is a polysorbate, a polyethoxylated castor oil derivative, a polyethoxylated fatty acid, a polyethoxylated alcohol, a polyoxyethylene-polyoxypropylene block copolymer, or an oxyethylated tertiary octylphenol formaldehyde polymer.

41. The method of claim 39 or 40, wherein the surfactant forms about 0.01% to about 20% weight/weight of the composition.

42. The method of claim 38, wherein the surfactant forms about 0.05% to about 10% weight/weight of the composition.

43. The method of any one of claims 1 to 42, wherein the composition further comprises a tonicity adjusting agent, a buffering agent, or a combination thereof.

44. The method of any one of claims 1 to 43, wherein the composition is buffered at a pH of 2.5 to 7.5.

45. The method of any one of claims 1 to 44, wherein the composition comprises 5 micrograms (ug) to 10,000 ug of the peptide.

46. The method of any one of claims 1 to 44, wherein the composition comprises at least 10 micrograms (ug), at least 25 ug, at least 50 ug, at least 100 ug, at least 150 ug, at least 200 ug, or at least 250 ug of the peptide.

47. The method of any one of claims 1 to 44, wherein the composition comprises 10 micrograms (ug), 25 ug, 50 ug, 100 ug, 150 ug, 200 ug, or 250 ug of the peptide.

48. The method of any one of claims 1 to 44, wherein the peptide is present at a concentration 0.1 milligrams per milliliter (mg/mL) to 10.0 mg/mL.


SEQUENCE LISTING (12 sequences)

SEQ ID NO: 1 - protein, 12 aa, Artificial Sequence
  Description of Artificial Sequence Synthetic peptide
HHIYLGAVNYIY

SEQ ID NO: 2 - protein, 12 aa, Artificial Sequence
  Description of Artificial Sequence Synthetic peptide
  MOD_RES (1)..(12): D-amino acid
YIYNVAGLYIHH

SEQ ID NO: 3 - protein, 12 aa, Artificial Sequence
  Description of Artificial Sequence Synthetic peptide
HHIYLGAVNYIY

SEQ ID NO: 4 - protein, 12 aa, Artificial Sequence
  Description of Artificial Sequence Synthetic peptide
  MOD_RES (1)..(12): D-amino acid
YIYNVAGLYIHH

SEQ ID NO: 5 - protein, 12 aa, Artificial Sequence
  Description of Artificial Sequence Synthetic peptide
  MOD_RES (1)..(12): D-amino acid
YIYNVAGLYIHH

SEQ ID NO: 6 - protein, 12 aa, Artificial Sequence
  Description of Artificial Sequence Synthetic peptide
  MOD_RES (1)..(12): D-amino acid
YVYNVAGLYVHH

SEQ ID NO: 7 - protein, 12 aa, Artificial Sequence
  Description of Artificial Sequence Synthetic peptide
  MOD_RES (1)..(1): Desamino-Tyr
  MOD_RES (1)..(12): D-amino acid
YVYNVAGLYVHH

SEQ ID NO: 8 - protein, 12 aa, Artificial Sequence
  Description of Artificial Sequence Synthetic peptide
  MOD_RES (1)..(1): Hydroxy-desamino-Tyr
  MOD_RES (1)..(12): D-amino acid
YIYNVAGLYIHH

SEQ ID NO: 9 - protein, 12 aa, Artificial Sequence
  Description of Artificial Sequence Synthetic peptide
  MOD_RES (1)..(1): Desamino-Tyr
  MOD_RES (1)..(12): D-amino acid
YVYNVAGLYVHH

SEQ ID NO: 10 - protein, 12 aa, Artificial Sequence
  Description of Artificial Sequence Synthetic peptide
  MOD_RES (1)..(12): D-amino acid
YVYNVAGLYVHH

SEQ ID NO: 11 - protein, 12 aa, Artificial Sequence
  Description of Artificial Sequence Synthetic peptide
  MOD_RES (1)..(12): D-amino acid
YIYNVAGLYIHH

SEQ ID NO: 12 - protein, 12 aa, Artificial Sequence
  Description of Artificial Sequence Synthetic peptide
  MOD_RES (1)..(12): D-amino acid
YVYNVAGLYVHH